(19) United States
(12) Patent Application Publication
(10) Pub. No.: US 2009/0269772 A1
(43) Pub. Date: Oct. 29, 2009

(54) SYSTEMS AND METHODS FOR IDENTIFYING COMBINATIONS OF COMPOUNDS OF THERAPEUTIC INTEREST

(76) Inventors: Andrea Califano, New York, NY (US); Riccardo Dalla-Favera, New York, NY (US); Owen A. O'Connor, New York, NY (US)

Correspondence Address: JONES DAY, 222 EAST 41ST ST, NEW YORK, NY, 10017, US

(21) Appl. No.: 12/432,579

(22) Filed: Apr. 29, 2009

Related U.S. Application Data
(60) Provisional application No. 61/048,875, filed on Apr. 29, 2008.
(60) Provisional application No. 61/061,573, filed on Jun. 13, 2008.

(51) Int. Cl. C12Q 1/68; C12Q 1/02; G06N 5/02

(52) U.S. Cl. 435/6; 435/29; 706/54; 707/E17.014

(57) ABSTRACT

Systems, methods, and apparatus for searching for a combination of compounds of therapeutic interest are provided. Cell-based assays are performed, each cell-based assay exposing a different sample of cells to a different compound in a plurality of compounds. From the cell-based assays, a subset of the tested compounds is selected. For each respective compound in the subset, a molecular abundance profile from cells exposed to the respective compound is measured. Targets of transcription factors and post-translational modulators of transcription factor activity are inferred from the molecular abundance profile data using information theoretic measures. This data is used to construct an interaction network. Variances in edges in the interaction network are used to determine the drug activity profile of compounds in the subset of compounds. The drug activity profiles are used to form a filter set of compound combinations from the subset of compounds.

### CROSS REFERENCE TO RELATED APPLICATIONS

This application claims benefit, under 35 U.S.C. § 119(e), of U.S. Provisional Patent Application No. 61/048,875, filed on Apr. 29, 2008, which is hereby incorporated by reference herein in its entirety. This application also claims benefit, under 35 U.S.C. § 119(e), of U.S. Provisional Patent Application No. 61/061,573, filed on Jun. 13, 2008, which is hereby incorporated by reference herein in its entirety.

### 1 FIELD

Computer systems and methods for determining combinations of compounds of therapeutic interest are provided.

### 2 BACKGROUND

Despite what appears to be a plethora of new drugs making their way to the clinic, there is a rapidly emerging crisis in traditional drug development for malignant diseases. The crisis is triggered by a paucity of new or lead drugs in the pipeline of most pharmaceutical companies. Large pharmaceutical firms have the means to generate many new potential lead compounds. Applications for increasingly smaller percentage of drugs are submitted to the United States Food and Drug Administration (FDA) for approval over time because many of these drugs have not been developed in a manner that respects the underlying systems biology perspective. It is also becoming increasingly clear that high-throughput screening approaches have exhausted the opportunities to focus strictly on single drug target candidates. As a result, pharmaceutical and biotech companies are being trapped between the demand for new blockbuster drugs that work on every patient and the dramatically smaller niches of diseases that are traceable to a common molecular mechanism.

A solution to the paucity of new or lead drugs in the pipeline is to develop combinations of compounds that include known drugs or other compounds of pharmaceutical interest. To understand the potential of combinatorial therapy, consider a simple metaphor. A possible way to block airline traffic in the United States is to disrupt an individual major air-traffic hub that routes a large number of planes. However, based on the airlines' ability to quickly re-route planes, air-traffic could be easily re-balanced, causing only moderate delays. This is akin to the traditional single drug-single target approach and a major reason why it has not been as successful as expected in the fight against some diseases, such as cancer. A combination target approach would rather target several major hubs simultaneously. In that case, even partial disruption would quickly produce a complete air-traffic paralysis, which could not be easily remedied.

Thus, as the above metaphor illustrates, combination therapy is a highly promising approach for many diseases of interest, such as cancer. In most cancer types, genetic alterations affect multiple pathways involved in pathogenesis, and therefore are not easily treated with a single drug. Emerging combination drugs regimens target multiple synergistic pathways to overcome the cancer cell redundant defensive mechanisms. Such combination regimens include drugs that, while toxic or ineffective in isolation, become safer and highly effective when administered in combination (combinatorial therapy). Specific drug combinations, in fact, can have minimal side effects on normal cells as they affect molecular targets that are cancer cell-specific. Furthermore, combinatorial therapies constitute a direct and unique opportunity to implement personalized medicine strategies, as the ability to selectively modulate the key pathways involved in pathogenesis provides great flexibility to address disease heterogeneity and population-specific effects. Some promising examples of combination therapy are already starting to emerge, including for instance the use of histone deacetylase (HDAC) inhibitors in combination with traditional anti-cancer drugs.

Combination therapy is further advantageous because it provides methods for identifying combinations of compounds that bypass cellular control redundancy. By inhibiting multiple, synergistic pathways, it is possible to bypass the natural redundancy of the cell control mechanisms that make many disease states resilient to a wide variety of single drug therapies. Thus, rather than having to inhibit or augment a single pathway with high doses of an individual drug, it is possible to target multiple interacting pathways in a synergistic fashion. This approach has particular efficacy for drug development for malignant diseases, such as cancer, which are characterized by defects in multiple signaling pathways, and are not easily treated with a single drug.

Combination therapy further has the potential for providing an exponential increase in therapeutic agents. The number of possible targets grows exponentially with the number of compounds used in combination, providing a vast array of potential targets. Where there may only be one target capable of inhibiting a specific cellular pathway, there may be hundreds of target combinations that may achieve the same goal and in a much more specific context. Hence, a whole new space of previously untapped therapeutic potential will become available.

Combination therapy further has the potential for yielding higher cellular specificity thereby reducing toxicity. By focusing on a single pathway it is unlikely to be effective in treating some diseases, such as cancer. In addition, while this focus on a single target in the cell may have some therapeutic merit, it is also likely to affect a larger number of healthy cells. On the other hand, the therapeutic index obtained from focusing on a set of specific pathways associated with a target disease, such as cancer, should reduce the toxicity against normal cells, while augmenting the efficacy against the malignant cells. This ability to identify the critical signaling ‘hubs’ in cells representative of a diseased state offers unique opportunities to both lower toxicity and improve efficacy. Adverse side effects are one of the primary causes contributing to the failure of clinical trials, often limiting how much therapy a patient can receive. Additionally, it is estimated that the cost of side effects to the health systems in the United States alone is in excess of $60 billion. For these reasons, it is expected that combinatorial therapy is an important avenue to personalized medicine where treatment specificity is mapped to a specific disease or tailored to the individual genetic profile (e.g. presence or absence of a specific pathway target or target mutation).

Still another advantage of combination therapy is the potential for lower doses. Use of synergistic pathway inhibitors will result in much smaller drug concentration requirement and thus lower toxicity.

As used herein, in some embodiments, synergistic behavior means that the combination of two or more drugs produces an effect in a biological organism that is greater than the effect that any one of the component drugs, when administered individually, has on the biological organism. As used herein, in some embodiments, synergistic behavior means that the combination of two or more drugs produces an effect in a biological organism that is greater than the sum of the individual effects that the component drugs, when administered individually, have on the biological organism. Thus, regardless of the embodiment of synergistic behavior adopted, very small concentrations of two or more drugs may achieve a more potent effect than a high concentration of any one drug by itself using the disclosed methods.

While the advantages of a properly implemented combination therapy strategy are apparent, there are also difficulties, which include the very large search space that must be searched in order to identify efficacious combinations. For example, if 100,000 compounds were to be screened for all possible two drug or three drug combination therapies, a total of 10,000,000,000 (ten billion) or 1,000,000,000,000,000 (one quadrillion) combinations, respectively, may have to be tested biochemically in vivo. Even with available robotic screening approaches, this is clearly not feasible. Yet, current libraries of compounds easily exceed 100,000 compounds. Another difficulty with combination therapy development is the poor generality of drug combinations. In some instances, such massive screening would have to be performed in several disease tissues because pathway availability varies significantly from tissue to tissue and individual to individual and thus results from one screening may not generalize. Furthermore, for each respective combination of compounds, several different concentrations (dosages) of each component compound in the respective combination would need to be tested. Since each of these different dosages must constitute a different assay, this need to explore dosage space effectively increases the number of combinations of compounds by several orders of magnitude that should be tested in order to adequately sample the compound combination space. Furthermore, at least two different cell lines are exposed to each respective combination of compounds at each of the respective concentrations (dosages) under study. For instance, one of these cell lines is representative of the disease under study and another of these cell lines is a control cell line that does not have the phenotype (e.g., disease or some other biological feature) under study. This would be necessary to assess the specificity of the compound combination, that is, its ability to affect disease tissue while not affecting normal tissue. Furthermore, in some instances, time delay, the time after treatment at which a cell line is assayed for a specific end-point phenotype, such as cell death, is preferably varied. For instance, in one cell-based exposure to a compound combination, the end-point phenotype is assayed ten hours after exposure to the compound combination whereas in another cell-based exposure to the very same compound combination, the end-point phenotype is assayed twenty hours after exposure to the compound combination. Given these drawbacks with combination therapy development it is evident that, although such combinatorial therapy is highly promising, currently available “brute force” robotic platforms cannot efficiently process the inordinately large number (˜10¹³ assuming only compound pairs) of cell-based assays, where such cell-based assays sample different compound combinations at varying compound concentrations in multiple cells lines using a plurality of different time delays, that would need to be tested in an exhaustive approach in order to identify useful compound combinations needed for such a therapeutic approach.

Given the above-background, what are needed in the art are improved systems and methods for identifying compound combinations of therapeutic interest.

Discussion or citation of a reference herein will not be construed as an admission that such reference is prior art.

### 3 SUMMARY

Recent advances in systems biology have shown that synergistic pathways and corresponding targets can be efficiently and systematically mapped in specific cellular contexts. This is achieved though perturbation studies using libraries of small chemical compounds. Similarly, it has been shown that perturbation studies using chemical compound libraries can also help identify the specific pathways and even targets affected by an individual compound (e.g.: assigning an “address” to a compound). One aspect combines these two approaches to concurrently identify (a) proteins in synergistic pathways whose inhibition would produce the desired end-point phenotype, and (b) compounds able to target these proteins. A second aspect involves using perturbation based on these compounds to directly identify compounds that can implement the desired end-point phenotype. Given a specific end-point phenotype, the systems and methods disclosed herein may reduce the number of potential synergistic compounds from >10¹⁰ to a few thousand that can be efficiently screened in experimental assays under a multitude of concentrations, delays, and other experimental conditions. Furthermore, since the target biology can be further investigated using available databases mapping tissue specific expression, a handful of candidate combinations can be selected such that they maximize availability in the diseased tissue while minimizing availability in other healthy tissues. In some embodiments, the inventive strategy is complemented by a traditional high-throughput screening assay approach in which individual compounds that show some potential towards the desired end-point phenotype are identified, and which may be further combined with compounds emerging from the bioinformatics screening. The novel combination of bioinformatics with a standardized high-throughput screening strategy allows for the search a significantly bigger space of potential drug combinations that are likely to have a higher probability of success. The novel platform described herein for the development of combinatorial therapies against diseases, such as cancer, allows for the rapid develop of multiple promising drug combinations and also allows for the generation of revenue from services provided to pharmaceutical and biotechnology companies.

An aspect provides a unique end-to-end systems biology discovery pipeline, which can identify multiple synergistic vulnerabilities of the cell that are representative of a disease state, such as cancer, and target such cells concurrently through the use of highly specific drug “cocktails.” This therapeutic paradigm provides a novel combination of traditional in vitro and in vivo target screening assays (e.g., high-throughput assays) with in silico (computational) screening assays that can identify the set of molecular targets in a given cell type. Target combinations can then be prioritized in silico and screened in vivo to produce highly tailored, less toxic and more efficacious therapeutic regimens for diseases of interest, such as cancer. By the novel integration of computational algorithms with automated screening assays, one aspect of the disclosed systems and methods reduces the number of potential compound combinations that need to be assayed from astronomical numbers such as 10¹⁰ compound combinations to about 10³ compound combinations. This reduced number of compound combinations provides an ideal size for experimental testing and prioritization of the drug combinations for pre-clinical and clinical validation. Accordingly, the ability to identify new combinations of drug regimens to treat diseases is significantly enhanced.

One aspect provides a method of searching for a combination of compounds of therapeutic interest. The method comprises performing a plurality of cell-based assays. In some embodiments, each cell-based assay in the plurality of cell-based assays comprises (i) exposing a different cell sample from a plurality of cell samples to a different compound in a plurality of compounds and (ii) measuring a phenotypic end-point phenotype in the cell sample upon exposure to the compound, thereby obtaining a plurality of phenotypic results. Each phenotypic result in the plurality of phenotypic results corresponds to a specific compound in the plurality of compounds. In some embodiments, control cell sample assays in which phenotypic results from cell samples that have been exposed only to the different type of media (e.g., DMSO) used to administer the compound are also performed. In some embodiments, a phenotypic result is cell death as a function of compound concentration (e.g., IC₅₀). In the method, based on the plurality of phenotypic results, a subset of compounds in the plurality of compounds that implement a desired end-point phenotype is determined. For instance, in some embodiments, a compound is deemed to implement a desired end-point phenotype if the compound kills cells representative of a diseased state at a concentration that is less than a concentration at which the compound kills cells that are representative of a control (non-diseased) state.

Once a subset of compounds has been thus identified, for each respective compound in the subset of compounds, a molecular abundance profile (MAP) assay is performed using a new cell sample treated with the respective compound, thereby obtaining a plurality of MAPs. An MAP comprises a plurality of measurements of the abundance of specific “cellular constituents” in a specific cell sample. As used herein, the term “cellular constituent” comprises a gene, a protein (e.g., a polypeptide, a peptide), a proteoglycan, a glycoprotein, a lipoprotein, a carbohydrate, a lipid, a nucleic acid, an mRNA, a cDNA, an oligonucleotide, a microRNA, a tRNA, or a protein with a particular modification. Thus, the term cellular constituent comprises a protein encoded by a gene, an mRNA transcribed from a gene, any and all splice variants encoded by a gene, cRNA of mRNA transcribed from a gene, any nucleic acid that contains the nucleic acid sequence of a gene, or any nucleic acid that is hybridizable to a nucleic acid that contains the nucleic acid sequence of a gene or mRNA translated from a gene under standard microarray hybridization conditions. Furthermore, an “abundance value” for a cellular constituent (cellular constituent abundance value) is a quantification of an amount of any of the foregoing, an amount of activity of any of the foregoing, or a degree of modification (e.g., phosphorylation) of any of the foregoing. As used herein, a gene is a transcription unit in the genome, including both protein coding and noncoding mRNAs, cDNAs, or cRNAs for mRNA transcribed from the gene, or nucleic acid derived from any of the foregoing. As such, a transcription unit that is optionally expressed as a protein, but need not be, is a gene. The abundance values used in the claim methods do not all have to be of the same class of abundance values. For example, in some embodiments, a single MAP can include amounts of mRNA, amounts of cDNA, amounts of protein, amounts of metabolites, activity levels of proteins, and/or all degrees of chosen modification (e.g., phosphorylation of proteins, etc.). In some embodiments, a MAP comprises a plurality of messenger RNA abundance measurements obtained by gene expression profile (GEP) microarrays. Each MAP in the plurality of MAPs comprises cellular constituent abundance values for a plurality of cellular constituents in a sample of cells that has been exposed to a compound in the subset of compounds.

One or more transcriptional targets of each of one or more expressed transcription factors are inferred from the MAP data. This can be accomplished using several approaches. In one such approach, for instance, regulation of a cellular constituent in the plurality of cellular constituents that are a transcriptional target by another cellular constituent in the plurality of cellular constituents that are transcription factors is inferred from an information theoretic measure I(X; Y) (e.g., mutual information) between the set of cellular constituent abundance values X for the transcription factor cellular constituent and the set of cellular constituent abundance values Y for the target cellular constituent in the MAP data. Here, X={xᵢ, . . . , xₙ} and each Xᵢ in X comprises data for the abundance of the transcription factor cellular constituent in the i-th GEP in the plurality of GEPs, and Y={yᵢ, . . . , yₙ} where each Yᵢ in Y comprises data for the abundance of the target cellular constituent in the i-th MAP in the plurality of MAPs, and n is an integer greater than one.

One or more transcription factor modulatory interactions, caused by one or more cellular constituents in the plurality of cellular constituents that are post-translational modulators of transcription factor activity, are also inferred from the MAP data. Specifically, for a cellular constituent gₘ that is a candidate post-translational modulator of the ability of a transcription factor cellular constituent g_{TF} to regulate a cellular constituents g_{T} that is a target of the transcription factor g_{TF}, this inferring comprises: (i) partitioning the plurality of MAPs into a first profile subset Lₘ⁺ and a second profile subset Lₘ⁻ in which gₘ is respectively at its highest (gₘ⁺) and lowest (gₘ⁻) abundances in the plurality of MAPs, where Lₘ⁻ and Lₘ⁺ are nonoverlapping and where Lₘ⁻ and Lₘ⁺ collectively encompass all or a portion (e.g., thirty percent or more, fifty percent or more, or more, seventy percent or more) of the MAPs in the plurality of MAPs, and (ii) identifying a conditional coregulation between g_{TF} and gₜ given gₘ by the gₘ dependent change in information difference ΔI(g_{TF},gₜ|gₘ) where

ΔI(g_{TF},gₜ|gₘ)=|I(g_{TF},gₜ|gₘ⁺)−I(g_{TF},gₜ,gₘ⁻)|

and where I(g_{TF},gₜ|gₘ⁺) is an information theoretic measure (e.g., correlation, degree of similarity, mutual information, etc.) between the abundance of the transcription factor g_{TF} and the abundance of the target g_{T} in the subset Lₘ⁺ of the MAPs, where gₘ is most abundant; and I(g_{TF},gₜ|gₘ⁻) is an information theoretic measure (e.g., correlation, degree of similarity, mutual information, etc.) between the abundance of the transcription factor g_{TF} and the abundance of the target g_{T} in the subset Lₘ⁻ of the MAPs, where gₘ is least abundant.

The method continues by forming an interaction network comprising one or more transcriptional interactions between one or more transcription factors and one or more transcription factor targets, as well as one or more modulatory interactions between one or more post-translational modulators of transcription factor activity and one or more transcription factors. The drug activity profile of each compound in the subset of compounds is then determined using the interaction network. Then, a filtered set of compound combinations comprising a plurality of compound combinations, each compound combination consisting of a combination of compounds in the subset of compounds is formed. A compound combination in the plurality of compound combinations is selected from the subset of compounds based on the drug activity profile of the each compound in the compound combination. For example, in some embodiments, the drug activity profile of a first compound includes one or more cellular constituents that are not in the drug activity profile of the second compound. In another example, in some embodiments, the drug activity profile of the first compound includes a cellular constituent that is in a first biological pathway in the interaction network while the drug activity profile of the second compound does not include any cellular constituent in this first biological pathway. In still another example, in some embodiments, the drug activity profile of the first compound includes a cellular constituent that is in a first biological pathway in the interaction network, the drug activity profile of the second compound does not include any cellular constituent in the first biological pathway and, correspondingly, the drug activity profile of the second compound includes a cellular constituent that is in a second biological pathway in the interaction network, and the drug activity profile of the first compound does not include any cellular constituent in the second biological pathway. Optionally, in some embodiments, the method further comprises screening a subset of compound combinations in the filter set of compound combinations for activity against the desired end-point phenotype, for example, using cell-based assays where cells are exposed to varying concentrations of compound combinations in the filter set of compound combinations. Optionally, in some embodiments, the method further comprises outputting the filter set of compound combinations to a display or a computer readable media.

The formation of a filter set of compound combinations comprising a plurality compound combinations, each compound combination consisting of a combination of compounds in a subset of compounds, where a first compound and a second compound in a first compound combination in the plurality of compound combinations is selected from the subset of compounds based on a difference between a drug activity profile of the first compound and a drug activity profile of the second compound has substantial practical application. The filter set of compound combinations substantially reduces the number of combinations that must be screened to identify a synergistic effect. As such the filter set of compounds reduces the costs of screening for suitable drug combinations.

### 4 BRIEF DESCRIPTION OF THE DRAWINGS

FIG. 1 shows an exemplary computer system for determining combinations of compounds of therapeutic interest.

FIG. 2 illustrates an exemplary method for determining combinations of compounds of therapeutic interest.

FIG. 3 illustrates an exemplary method for determining combinations of compounds of therapeutic interest.

FIG. 4 illustrates cell-based assays, in accordance with the prior art, that can be used in the methods disclosed herein.

Like reference numerals refer to corresponding parts throughout the several views of the drawings.

### 5 DETAILED DESCRIPTION

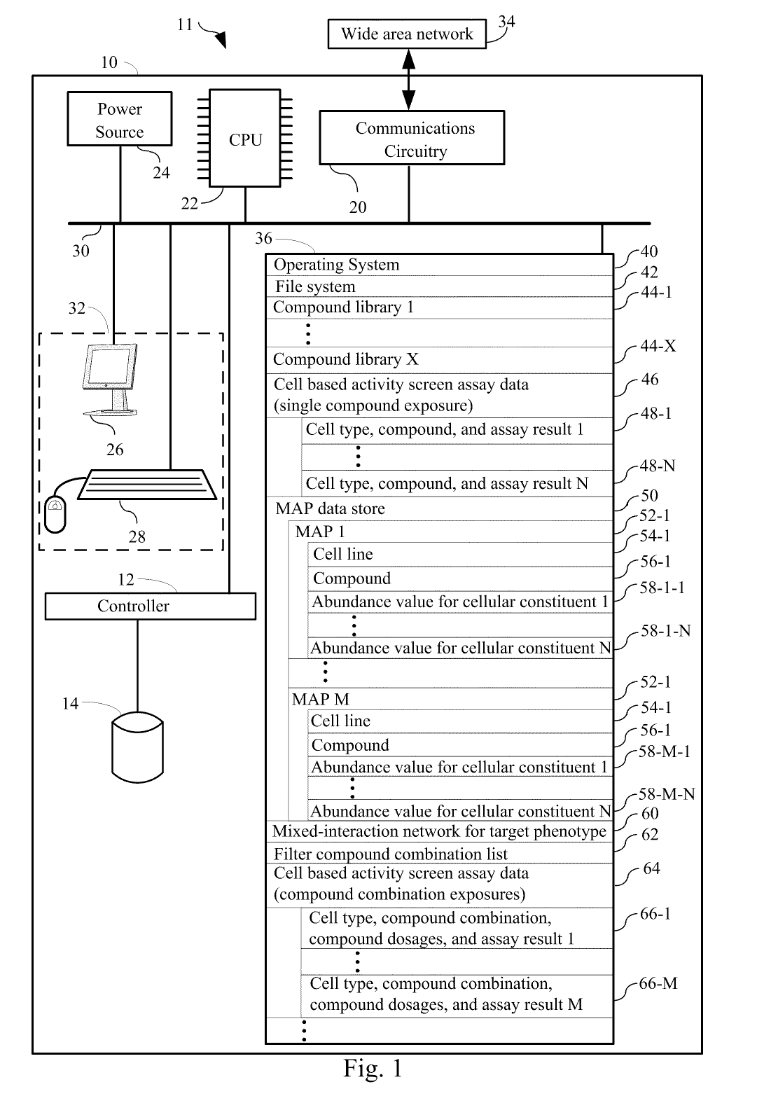

FIG. 1 details an exemplary system 11 for use in determining combinations of compounds of therapeutic interest. The system preferably comprises a computer system 10 having:
a central processing unit 22;
a main non-volatile storage unit 14, for example a hard disk drive, for storing software and data, the storage unit 14 controlled by storage controller 12;
a system memory 36, preferably high speed random-access memory (RAM), for storing system control programs, data, and application programs, comprising programs and data loaded from non-volatile storage unit 14; system memory 36 may also include read-only memory (ROM);
a user interface 32, comprising one or more input devices (e.g., keyboard 28, a mouse) and a display 26 or other output device;
a network interface card 20 (communications circuitry) for connecting to any wired or wireless communication network 34 (e.g., a wide area network such as the Internet);
a power source 24 to power the aforementioned elements; and
an internal bus 30 for interconnecting the aforementioned elements of the system.

Operation of computer 10 is controlled primarily by operating system 40, which is executed by central processing unit 22. Operating system 40 can be stored in system memory 36. In a typical implementation, system memory 36 also includes:
a file system 42 for controlling access to the various files and data structures used herein;
one or more compound libraries 44 (e.g., a general purpose library of compounds, a library of compounds with known targets, and/or a library of compounds that have been approved by a regulatory agency such as the Food and Drug Administration, etc.);
cell based activity screen assay data 46 from cell based assays in which individual compounds from one or more of the compound libraries are exposed cell lines thereby resulting in assay result data 48;
a MAP data store 50 that comprises MAPs 52 for each compound of interest 56 in a cell line 54, each 52 comprising cellular constituent abundance data 58 for a plurality of cellular constituents;
a mixed-interaction network 60 for a target phenotype comprising protein-protein interactions, protein-DNA interactions and transcription factor modulatory interactions that occur in a cell line that is representative of (exhibits) a phenotypic trait under study; and
a filter compound combination list 62 comprising combinations of compounds from compound libraries 44 selected based on, for example, complementarity in drug pathways affected by such compounds and compound selectivity in the mixed-interaction network 60 for the target phenotype; and
cell based activity screen assay data 46 from cell based assays in which cell lines are treated with individual compounds from one or more of the compound libraries, thereby resulting in assay result data 48.

In some embodiments, memory 36 further comprises the drug activity profile of each of the compounds for which there is MAP data. Such drug activity profile data provides and indication of which genes in the mixed-interaction network 60 for the target phenotype are affected by such drugs.

As illustrated in FIG. 1, computer 10 comprises compound libraries 44, cell based activity screen data 46 (single compound exposure), a MAP data store 50, a mixed-interaction network 60 for a target phenotype, a filter compound combination list 62, an cell based activity screen data 64 (compound combination exposures). Such data can be in any form including, but not limited to, a flat file, a relational database (SQL), or an on-line analytical processing (OLAP) database (MDX and/or variants thereof). In some specific embodiments, such data is stored in a hierarchical OLAP cube. In some specific embodiments, such data is stored in a database that comprises a star schema that is not stored as a cube but has dimension tables that define hierarchy. Still further, in some embodiments, such data is stored in a data structure that has hierarchy that is not explicitly broken out in the underlying database or database schema (e.g., dimension tables that are not hierarchically arranged). In some embodiments, such data is stored in a single database. In other embodiments, such data is in fact stored in a plurality of databases that may or may not all be hosted by the same computer 10. In such embodiments, some of the data illustrated in FIG. 1 as being stored in memory 36 is, in fact, stored on computer systems that are not illustrated by FIG. 1 but that are addressable by wide area network 34.

In some embodiments, the data illustrated in memory 36 of computer 10 is on a single computer (e.g., computer 10) and in other embodiments the data illustrated in memory 36 of computer 10 is hosted by several computers (not shown). In fact, all possible arrangements of storing the data illustrated in memory 36 of computer 10 on one or more computers can be used so long as these components are addressable with respect to each other across computer network 34 or by other electronic means. Thus, a broad array of computer systems can be used.

As depicted in FIG. 1, in typical embodiments, each MAP 52 is associated with the cell type 54 of the sample that was used to construct the MAP 52. Each MAP 52 further comprises the abundance values 58 for a plurality of cellular constituents. Further, each MAP 52 optionally indicates a compound 56 from one of the compound libraries 44 that the cell line 54 was treated with, prior to obtaining the MAP data. In such embodiments, the MAP 52 may further include the concentration of the compound to which the cell line 54 was exposed prior to obtaining the microarray data.

In some embodiments, the abundance value for a cellular constituent is determined by a degree of modification of a cellular constituent that is encoded by or is a product of a gene (e.g., is a protein or RNA transcript). In some embodiments, a cellular constituent is virtually any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite) and/or any other variable cellular component or protein activity, degree of protein modification (e.g., phosphorylation), or a discriminating molecule or discriminating fragment of any of the foregoing, that is present in or derived from a biological sample that is modified by, regulated by, or encoded by a gene.

A cellular constituent can, for example, be isolated from a biological sample from a member of the first population, directly measured in the biological sample from the member of the first population, or detected in or determined to be in the biological sample from the member of the first population. A cellular constituent can, for example, be functional, partially functional, or non-functional. In addition, if the cellular constituent is a protein or fragment thereof, it can be sequenced and its encoding gene can be cloned using well-established techniques.

A cellular constituent can be an RNA encoding a gene that, in turn, encodes a protein or a portion of a protein. However, a cellular constituent can also be an RNA that does not necessarily encode for a protein or a portion of a protein. As such, a “gene” is any region of the genome that is transcriptionally expressed. Thus, examples of genes are regions of the genome that encode microRNAs, tRNAs, and other forms of RNA that are encoded in the genome as well as those genes that encode for proteins (e.g. messenger RNA).

In some embodiments, the cellular constituent abundance data for a gene is a degree of modification of the cellular constituent. Such a degree of modification can be, for example, an amount of phosphorylation of the cellular constituent. Such measurements are a form of cellular constituent abundance data. In one embodiment, the abundance of the at least one cellular constituent that is measured and stored as abundance value 50 for a cellular constituent comprises abundances of at least one RNA species present in one or more cells. Such abundances can be measured by a method comprising contacting a gene transcript array with RNA from one or more cells of the organism, or with cDNA derived therefrom. A gene transcript array comprises a surface with attached nucleic acids or nucleic acid mimics. The nucleic acids or nucleic acid mimics are capable of hybridizing with the RNA species or with cDNA derived from the RNA species.

### 5.1 Exemplary Method

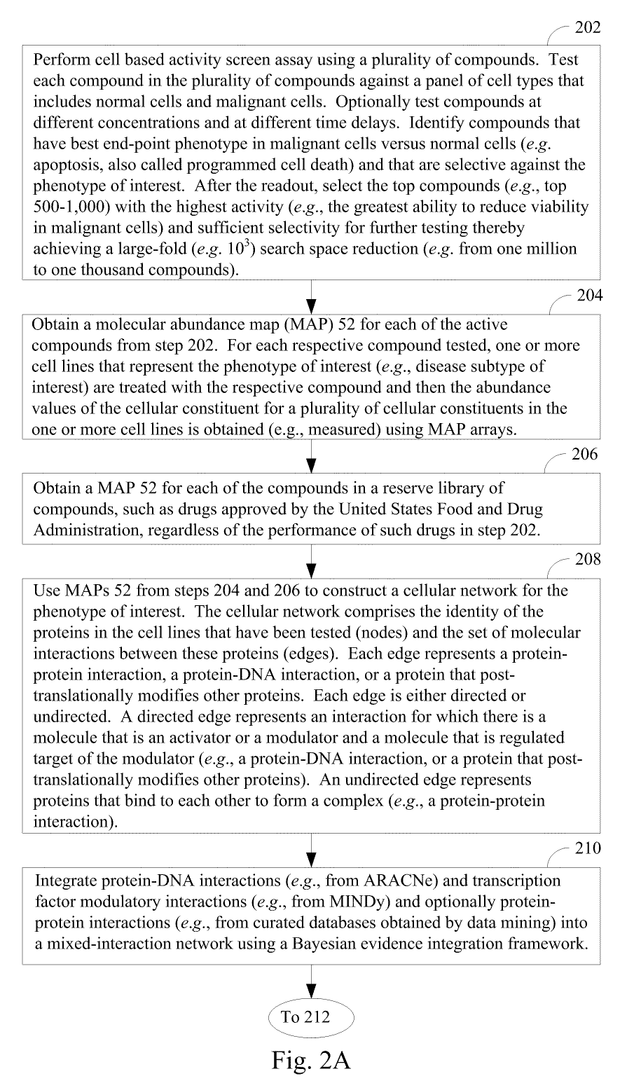

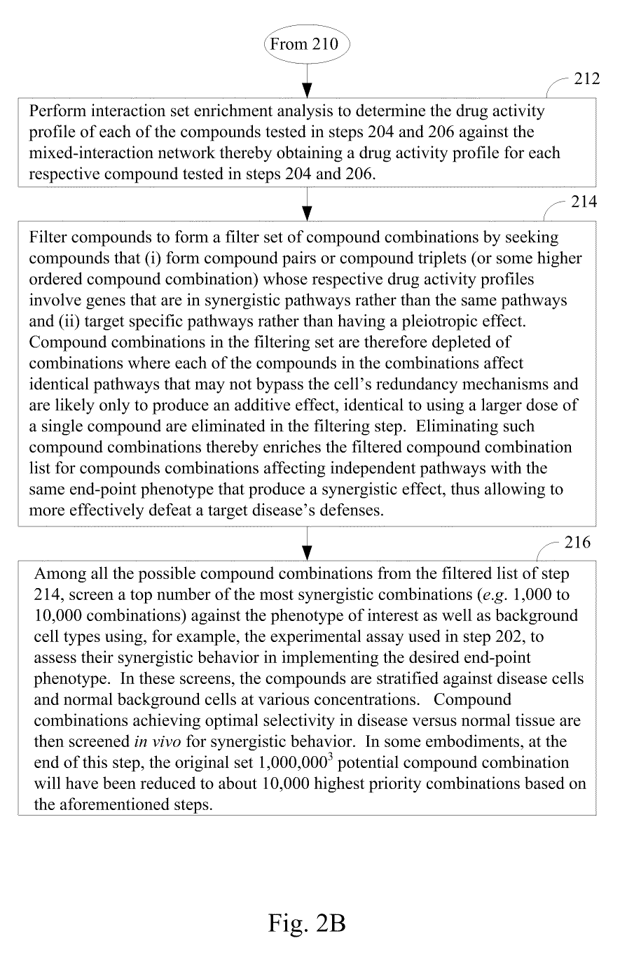

Referring to FIG. 2, an exemplary method for determining combinations of compounds of therapeutic interest is disclosed. Further, several variations of this exemplary method are disclosed in the following text.

Step 202. In step 202, compounds in one or more compound libraries are screened to assess their individual ability to achieve an end-point phenotype in malignant cells versus normal cells (e.g. apoptosis, also called programmed cell death).

In some embodiments such compound libraries include drugs approved by a regulatory agency such as the Food and Drug Administration of the United States, compounds that have known macromolecular targets, and/or other compounds of interest.

In some embodiments, a compound library screened in step 202 comprises five or more, ten or more, twenty or more, thirty or more, fifty or more, one hundred or more, two hundred or more, or five hundred or more of the compounds listed in Section 5.9.

In some embodiments, a compound library comprises compounds that have been approved under Section 505 of the Federal Food, Drug, and Cosmetic Act as set forth in Approved Drug Products with Therapeutic Equivalence Evaluations, 28^{th} Edition (the “Orange Book”), U.S. Department of Health and Human Services, Food and Drug Administration, Center for Drug Evaluation and Research, Office of Pharmaceutical Science, which is hereby incorporated by reference herein in its entirety for such purpose.

In some embodiments, a compound library comprises five or more, ten or more, twenty or more, thirty or more, fifty or more, one hundred or more, two hundred or more, or five hundred or more of the compounds in the spectrum collection offered by MicroSource Discovery Systems, Inc. (MDSI) (Gaylordsville, Conn.) and described in J Virology 77: 10288 (2003); and Ann Rev Med 56: 321 (2005), each of which is hereby incorporated by reference in its entirety.

In some embodiments, a compound in one or more compound libraries, diluted in a delivery medium (e.g. DMSO), is used to treat a sample of cells from a specific disease sub-phenotype and any combination of cell samples that represent non-disease tissue or other distinct sub-phenotypes of the disease under study. Then, the result that is measured is the difference in end-point phenotype in cells representative of the disease sub-phenotype of interest versus the other cell samples, either non-disease related or specific to a distinct disease sub-phenotype.

In some embodiments, a compound in one or more compound libraries, optionally diluted in a delivery medium (e.g. DMSO), is used to treat a sample of cells that is representative of a disease model of interest (e.g., a certain B cell line that represents a B cell specific disease). The phenotypic result that is measured for the compound in some embodiments is a relative abundance of each cellular constituent in a plurality of cellular constituents in the sample of cells (i) after exposure only to the delivery medium for a time t (e.g. 6 hours) and (ii) after exposure to the compound diluted in the delivery medium for the same time t. For instance, one aliquot of the cell sample that is representative of a phenotype of interest is used to measure abundance of a plurality of cellular constituents with exposure only to the delivery medium for a time t and another aliquot of the same cell sample is exposed to the respective compound, diluted in the delivery medium, for the same time t and then used to measure abundance of a plurality of cellular constituents. In this way, a differential profile for the respective compound can be computed. For example, consider the case in which there are 1000 cellular constituents that are deemed to be informative for the phenotype of interest. The abundance of all or a portion (e.g., at least fifty percent, at least seventy percent, etc.) of the 1000 cellular constituents are measured in a first aliquot of cells that are representative of a phenotype of interest treated only with the delivery medium for a time t (e.g., six hours). The abundance of all or a portion (e.g., at least fifty percent, at least seventy percent, etc.) of the 1000 cellular constituents are also measured in a second aliquot of cells that are representative of the phenotype of interest after the second aliquot of cells have been exposed to a predetermined amount of the respective compound (e.g., 1 nanomolar, diluted in the delivery medium) for the same time t. Then, the differential profile of the compound is given as the differential abundance of those cellular constituents that have been measured in both the first aliquot of cells and the second aliquot of cells.

In some embodiments multiple differential profiles are computed for a given compound. For example, in some embodiments, a differential profile is generated for each of several different time exposures, concentrations, or cell types. In one instance, the abundance of all or a portion (e.g., at least fifty percent, at least seventy percent, etc.) of a plurality of cellular constituents are measured in a first aliquot of cells that are representative of the phenotype of interest exposed only to the delivery medium for a time t₁ (e.g. six hours). Then, the abundance of all or a portion (e.g., at least fifty percent, at least seventy percent, etc.) of a plurality of cellular constituents are measured in a second aliquot of cells that are representative of the phenotype of interest exposed only to the delivery medium for a time t₂ (e.g. twelve hours). Then, the abundance of all or a portion (e.g., at least fifty percent, at least seventy percent, etc.) of a plurality of cellular constituents are measured in a third aliquot of cells after the third aliquot of cells has been exposed to a predetermined amount of the respective compound (e.g., 1 nanomolar, diluted in the deliver medium) for the time t₁. Then, the abundance of all or a portion (e.g., at least fifty percent, at least seventy percent, etc.) of a plurality of cellular constituents are measured in a fourth aliquot of cells after the fourth aliquot of cells has been exposed to a predetermined amount of the respective compound (e.g., 1 nanomolar, diluted in the deliver medium) for the time t₂. Then, a first differential profile of the compound is given as the differential abundance of those cellular constituents that have been measured in both the first aliquot of cells and the third aliquot of cells. Further, a second differential profile of the compound is given as the differential abundance of those cellular constituents that have been measured in both the second aliquot of cells and the fourth aliquot of cells.

In another example in which multiple differential profiles are computed for a given compound, a differential profile for the compound is generated in a cell type representative of the phenotype of interest ph₁ and in another distinct cell type representative of the phenotype ph₂ (e.g. non-disease related or presenting a different disease sub-phenotype). For example the abundance of all or a portion (e.g., at least fifty percent, at least seventy percent, etc.) of a plurality of cellular constituents are measured in a first aliquot of cells representative of the ph₁ phenotype exposed only to the delivery medium for a specific time t (e.g., six hours). Then, the abundance of all or a portion (e.g., at least fifty percent, at least seventy percent, etc.) of a plurality of cellular constituents are measured in a second aliquot of cells representative of the ph₁ phenotype after the second aliquot of cells has been exposed to a predetermined amount of the respective compound (e.g., 1 nanomolar, diluted in the deliver medium) for a time t. Further, the abundance of all or a portion (e.g., at least fifty percent, at least seventy percent, etc.) of a plurality of cellular constituents are measured in a third aliquot of cells representative of the ph₂ phenotype exposed only to the delivery medium for a time t. Then, the abundance of all or a portion (e.g., at least fifty percent, at least seventy percent, etc.) of a plurality of cellular constituents are measured in a fourth aliquot of cells representative of the ph₂ phenotype after the fourth aliquot of cells has been exposed to a predetermined amount of the respective compound (e.g., 1 nanomolar, diluted in the deliver medium) for a time t. Then, a first differential profile of the compound is given as the differential abundance of those cellular constituents that have been measured in both the first aliquot of cells and the second aliquot of cells. Further, a second differential profile of the compound is given as the differential abundance of those cellular constituents that have been measured in both the third aliquot of cells and the fourth aliquot of cells. In typical embodiments, the time t for each of the four measurements is the same or is approximately the same.

In some embodiments, each of the differential profiles for a given compound are combined together to form a combined differential profile for a given compound (e.g., by averaging differential abundance of like cellular constituents in each of the plurality of cellular constituent profiles for a given compound). In typical embodiments, each such differential profile is the differential profile of (i) a first aliquot of a cell type that is exposed only to delivery medium for a time t and (ii) a second aliquot of the cell type that is exposed to a compound in the delivery medium for a time t. In some embodiments, each of the differential profiles for a given compound are not combined together to form a combined differential profile for a given compound. In some embodiments, each of the differential profiles for a given compound that were performed using cell samples representative of the phenotype of interest are combined together to form a first combined differential profile for a given compound and each of the differential profiles for a given compound that were performed using cell samples not representative of the phenotype of interest are combined together to form a second combined differential profile for a given compound.

In some embodiments, the cells are of a tissue type that is appropriate for study of a disease of interest. For example, if the disease of interest is liver cancer, the cells that are assayed (exposed to compounds) could be cell lines derived from liver cancer biopsies or the actual biopsies from liver cancer biopsies. Exemplary cell types that are from specific tissues are disclosed in Section 5.2 below. In typical embodiments, the cell types that are exposed to compounds will include cell types that are representative of the phenotype (e.g., disease state) under study. Representative nonlimiting examples of disease states that may be studied using the methods disclosed herein are disclosed in Section 5.3 below.

In some embodiments, more than 1000 compounds, more than 5,000 compounds, more than 10,000 compounds, more than 25,000 compounds, more than 50,000 compounds, more than 100,000 compounds, more than 500,000 compounds or more than 1,000,000 compounds are screened in the cell based assays.

In some embodiments, compounds are screened robotically against cell lines representative of the biological phenotype of interest in step 202. In some embodiments, predefined compound concentrations are used. In some embodiments, only a single compound concentration (e.g., dosage) is used. In one example, what is meant by the term compound concentration is the concentration of the compound in the solution or other form of biomass that contains the cells being exposed to the compound. For instance, if the test cells being exposed to the compound are in a liquid cell media, the concentration of the compound is the total concentration of the compound in the liquid cell media holding the test cells. In some embodiments, each compound assayed in step 202 is assayed against test cells at a single concentration (e.g., 1 nanomolar, 100 nanomolar, 1 micromolar, or some other value). In some embodiments, each compound assayed in step 202 is assayed against test cells at two or more different concentrations, three or more concentrations, four or more concentrations, or between 5 and 100 concentrations. In some embodiments, each compound is tested against two different cell lines at five different concentrations, where one of the cell lines represents a nonmalignant state and the other cell line represents a malignant state of the disease of interest.

In some embodiments, each compound is assayed after different exposure times. Here, an exposure time refers to the period of time between when a cell line or other biological sample is first exposed to a compound and when the cell line or other biological sample is assayed for an end-point phenotype. In some embodiments, the range of exposure times that are sampled for a particular compound is dependent upon the phenotype under investigation. In some embodiments, the range of exposure times that are sampled for a particular compound ranges from between 1 second and 10 days, between 1 minute and 5 days, between 10 minutes and 3 days or some other range of time. In some embodiments, one or more exposure times, two or more exposure times, three or more exposure times, or five or more exposure times are assayed in a cell-based assay for each compound under study and for each compound concentration under study in step 202. Typically, a different aliquot of cells is used for each such exposure. For example, if two exposure times are of interest, four measurements are performed: the first measurement uses a first aliquot of the cell line or other biological sample exposed to the delivery medium without compound for a time t₁, the second measurement uses a second aliquot of the cell line or other biological sample exposed to the delivery medium with the compound of interest for the time t₁, the third measurement uses a third aliquot of the cell line or other biological sample exposed to the delivery medium without compound for a time t₂, and the fourth measurement uses a fourth aliquot of the cell line or other biological sample exposed to the delivery medium with compound for a time t₂. Further, in some embodiments for each such exposure time, compound, and compound concentration, several different cell-based assays are performed, where each such cell-based assay is against a different cell sample. Typically, for each such exposure time and compound, there is a corresponding measurement using an aliquot of the cell line or other biological sample with delivery medium in absence of any compound.

To assess the end-point phenotype in high-throughput fashion, fully automated fluorescent or luminescent readout is performed in some embodiments using standard robotically integrated plate-readers. In some embodiments, the fluorescent readout is proportional or otherwise indicative of the number of cells in a culture that are undergoing apotosis or that are viable. In some embodiments, after readout, the top 2,000 compounds, the top 1,000 compounds, the top 500 compounds or some other user specified upper threshold number of compounds with the highest activity (e.g., greatest ability to reduce viability in malignant cells) are selected for further analysis. In some embodiments, after readout, the top 2,000 compounds, the top 1,000 compounds, the top 500 compounds or some other user specified lower threshold number of compounds with the highest activity are selected for further analysis. Step 202 achieves about a 10³ fold search space reduction (e.g. from one million compounds to one thousand compounds) in some embodiments. More description of cell based assays that can be used for step 202 is provided in Section 5.7, below.

In some embodiments, any of the above-identified compound libraries screened in various implementations of step 202 comprise molecules that satisfy the Lipinski's Rule of Five: (i) not more than five hydrogen bond donors (e.g., OH and NH groups), (ii) not more than ten hydrogen bond acceptors (e.g. N and O), (iii) a molecular weight under 500 Daltons, and (iv) a LogP under 5. The “Rule of Five” is so called because three of the four criteria involve the number five. See, Lipinski, 1997, Adv. Drug Del. Rev. 23, 3, which is hereby incorporated herein by reference in its entirety. In some embodiments, compounds in the above-identified compound libraries satisfy criteria in addition to Lipinski's Rule of Five. For example, in some embodiments, the compounds have five or fewer aromatic rings, four or fewer aromatic rings, three or fewer aromatic rings, or two or fewer aromatic rings. In some embodiments, the molecules tested herein are any organic compound having a molecular weight of less than 2000 Daltons, of less than 4000 Daltons, of less than 6000 Daltons, of less than 8000 Daltons, of less than 10000 Daltons, or less than 20000 Daltons.

In some embodiments, step 202 comprises determining, from the plurality of phenotypic results obtained for the test compounds, a subset of compounds that implement the desired end-point phenotype. In some embodiments, this is accomplished by computing a similarity between the differential cellular constituent abundances of a differential profile of each compound to the differential cellular constituent abundances of a cellular constituent signature of the desired end-point phenotype. In some embodiments, this cellular constituent signature for the desired end-point phenotype is defined as the difference in cellular constituent abundance for a plurality of cellular constituents in (i) a cell sample representative of the phenotype of interest but not exhibiting a desired end-point phenotype (e.g., malignant but alive) and (ii) a cell sample representative of the phenotype of interest and also exhibiting the desired end-point phenotype (malignant and undergoing apoptosis). For example, consider the case in which there are a plurality of cellular constituents whose abundances are measured in (i) a first cell sample representative of the phenotype of interest in a normal malignant state (e.g., malignant cells that are alive) and (ii) a second cell sample representative of the phenotype of interest that is exhibiting a desired end-point phenotype (e.g., the phenotype of interest is malignant cells and the desired end-point phenotype is apoptosis). In this example, the cellular constituent signature for the desired end-point phenotype is the differential cellular constituent abundance of each cellular constituent, for a plurality of cellular constituents, between the first cell sample type and the second cell sample type.

In some embodiments, the similarity between the differential cellular constituent abundances of a differential profile of a compound and the differential cellular constituent abundances of a cellular constituent signature of the desired end-point phenotype is measured by a measure of similarity such as mutual information, a correlation, a T-test, a Chi² test, or some other parametric or nonparametric means. In some embodiments, the measure of similarity is adapted from any of the sixty-seven measures of similarity described in McGill, “An Evaluation of Factors Affecting Document Ranking by Information Retrieval Systems,” Project report, Syracuse University School of Information Studies, which is hereby incorporated by reference herein in its entirety. In some embodiments, the top 2,000 compounds, the top 1,000 compounds, the top 500 compounds or some other user specified upper threshold number of compounds with the (e.g. highest, best) similarity between the differential profile of the compound and the cellular constituent profile signature of the desired end-point phenotype are selected for further analysis.

Embodiments in which the end-point phenotype is apotosis have been disclosed. In other embodiments the desired end-point phenotype is cell proliferation (e.g., in a cancer model). In other embodiments the desired end-point phenotype is a predetermined molecular event (e.g., protein folding) that is monitored within a cell. In some embodiments, such a predetermined molecular event (e.g., protein folding) is monitored by fluorescence resonance energy transfer (FRET). FRET involves the direct transfer of energy from a donor to an acceptor molecule, which is detected by spectroscopy. For example, the green fluorescent protein deriviatives cyan (CFP) and yellow (YFP) fluorescent proteins are useful FRET donor/acceptor pairs in cell-based assays. In the case where CFP and YFP are used as the donor/acceptor, when the donor/acceptor distance exceeds approximately 80 Angstroms, no FRET occurs, and donor excitation produces an emission of only λ₁. The proximity of the donor/acceptor pair (less than 80 Angstroms) results in FRET upon donor excitation, and donor excitation produces a new emission of t. It is possible to measure this FRET signal quantitatively in an inteact cell. Thus, the fusion of proteins of interest to CFP and YFP allows quantitative detection of FRET based on protein interactions. Cells expressing these fusion proteins are cultured in a microtiter format, and the FRET signal is quantitatively measured by using a micrometer-bases fluorescence plate reader. See Jones and Diamond, 2007, ACS Chemical Biology 2, 718-724, which is hereby incorporated by reference herein in its entirety.

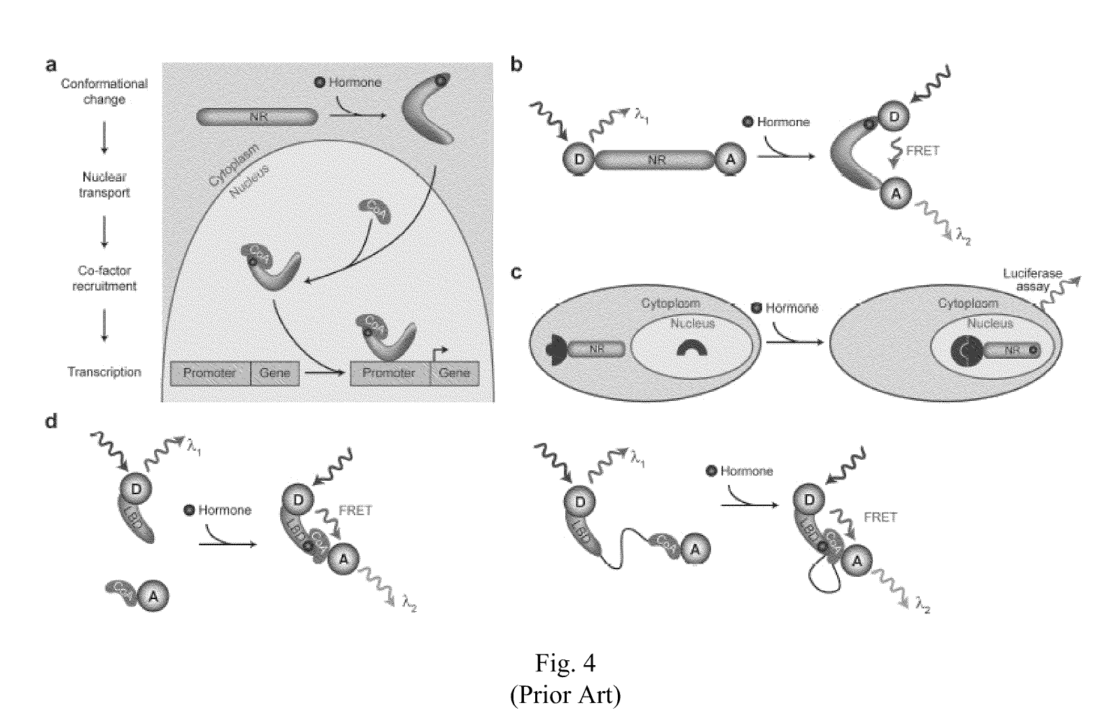

FRET signals have been used to measure the aggregation of misfolded proteins in neurodegeneration cell based models. See Pollitt et al., 2003, “A rapid cellular FRET assay of polyglutamine aggregation identifies a novel inhibitor, 2003, Neuron 40, 685-694, which is hereby incorporated by reference herein in its entirety. FIG. 4 illustrates additional forms of cell based assays that can be used to measure predetermined molecular events. In the case of FIG. 4, the protein under study is a nuclear receptor (NR). One of skill in the art will appreciate that, rather than studying a nuclear receptor, other proteins can be assayed using the teachings of FIG. 4. As illustrated in FIG. 4a) NRs undergo multiple steps of processing after ligand activation, which can produce nonspecific hits during a screen. To overcome this problem, as illustrated in FIG. 4b), the amino and carboxy termini of a NR is tagged with a FRET donor (D) and acceptor (A). Conformational change induced by hormone binding reduces the intramolecular distance and increases the FRET signal. Alternatively, as illustrated in FIG. 4c), the amino terminus of a NR is tagged with one-half of a luciferase enzyme. The second half is tagged with a nuclear localization sequence and is constitutively nuclear. Nuclear translocation of the NR allows reconstitution of the luciferase activity which can be quantitatively assayed in a cell based assayed. Alternatively, as illustrated in FIG. 4d), the LBD of a NR is tagged with a FRET donor, and a coactivator protein (CoA) is tagged with a FRET acceptor. Hormone binding induces intermolecular FRET. Alternatively, as further illustrated in FIG. 4d), a single fusion protein has a FRET donor fused to the LBD, fused in turn to a coactivator peptide motif, and then fused to a FRET acceptor. Hormone binding induces intramolecular FRET which can be measured quantitatively in a cell-based assay. See Jones and Diamond, 2007, ACS Chemical Biology 2, 718-724, which is hereby incorporated by reference herein in its entirety.

In some embodiments, the desired end-point phenotype is the appearance or disappearance of a FRET signal, a luciferase signal, or any other reporter signal from any of the assay formats disclosed herein. In some embodiments, the microarray cellular constituent abundance data described above is measured when this desired end-point phenotype is reached.

In some embodiments, the desired end-point phenotype is the attenuation or deattenuation of a FRET signal, a luciferase signal, or any other reporter signal from any of the assay formats disclosed herein. In some embodiments, the microarray cellular constituent abundance data described above is measured when this desired end-point phenotype is reached.

In some embodiments, the desired end-point phenotype is the measurement of a FRET signal, a luciferase signal, or any other reporter signal above a first threshold value from any of the assay formats disclosed herein. In some embodiments, the microarray cellular constituent abundance data described above is measured when this desired end-point phenotype is reached.

In some embodiments, the desired end-point phenotype is the measurement of a FRET signal, a luciferase signal, or any other reporter signal below a first threshold value from any of the assay formats disclosed herein. In some embodiments, the microarray cellular constituent abundance data described above is measured when this desired end-point phenotype is reached.

In some embodiments, the desired end-point phenotype is the selective read-through of a nonsenses codon, such as was the case in the cell base assay of Welch, 2007, Nature 447, 87-91, which is hereby incorporated by reference herein. In some embodiments, the microarray cellular constituent abundance data described above is measured when this desired end-point phenotype is reached.

Step 204. Molecular abundance maps (MAPs) 52 of active compounds from step 202 are obtained in step 204. For each respective compound tested, one or more cell lines are treated with the respective compound and then the abundance values of cellular constituents in the one or more cell lines are obtained using high throughput techniques such as gene expression profile microarrays. In some embodiments where a compound is exposed to cells at multiple concentrations, the smallest concentration to achieve a differential end-point phenotype in malignant cells versus normal cells is used in step 204. In some embodiments, where a compound is exposed to cells at multiple concentrations, the concentration used in step 204 is determined on a case by case basis upon review of data from step 202.

In some embodiments, MAPs 52 that are obtained in step 204 use microarray profiling techniques for transcriptional state measurements with any of the methods known in the art and/or those disclosed in Section 5.5 below. In some embodiments the microarray data is preprocessed using any preprocessing routine known in the art such as, for example any of the preprocessing techniques disclosed in Section 5.4. In some embodiments, each of the active compounds is exposed to two or more cell lines, three or more cell lines, five or more cell lines, or ten or more cell lines resulting in two or more MAPs, three or more MAPs, five or more MAPs, or ten or more MAPs. In some embodiments, each such MAP 52 is termed a “gene expression profile” herein.

In some embodiments, a MAP 52 comprises the cellular constituent abundance values from a microarray that is designed to quantify an amount of nucleic acid or ribonucleic acid (e.g. messenger RNA) in a cell line 54 or other biological sample after the cell line 54 or other biological sample has been exposed to test compound. Examples of microarrays that may be used include, but are not limited to, the Affymetrix GENECHIP Human Genome U133A 2.0 Array (Santa Clara, Calif.) which is a single array representing 14,500 human genes. The values in a MAP 52 are referred to as abundance values 58 as depicted in FIG. 1. In some embodiments, each MAP 52 comprises the cellular constituent abundance values from any Affymetrix expression (quantitation) analysis array including, but not limited to, the ENCODE 2.0R array, the HuGeneFL Genome Array, the Human Cancer G110 Array, the Human Exon1.0 ST Array, the Human Genome Focus Array, the Human Genome U133 Array Plate Set, the Human Genome U133 Plus 2.0 Array, the Human Genome U133 Set, the Human Genome U133A 2.0 Array, the Human Genome U95 Set, the Human Promoter 1.0R array, the Human Tiling 1.0R Array Set, the Human Tiling 2.0R Array Set, and the Human X3P Array.

In some embodiments, a MAP 52 comprises the cellular constituent abundance values from an exon microarray. Exon microarrays provide at least one probe per exon in genes traced by the microarray to allow for analysis of gene expression and alternative splicing. Examples of exon microarrays include, but are not limited to, the Affymetrix GENECHIP Human Exon1.0 ST array. The GENECHIP Human Exon1.0 ST array supports most exonic regions for both well-annotated human genes and abundant novel transcripts. A total of over one million exonic regions are registered in this microarray system. The probe sequences are designed based on two kinds of genomic sources, e.g. cDNA-based content that includes the human RefSeq mRNAs, GenBank and ESTs from dbEST, and the gene structure sequences which are predicted by GENSCAN, TWINSCAN, and Ensemble. The majority of the probe sets are each composed of four perfect match (PM) probes of length 25 bp, whereas the number of probes for about 10 percent of the exon probe sets is limited to less than four due to the length of probe selection region and sequence constraints. With this microarray platform, no mismatch (MM) probes are available to perform data normalization, for example, background correction of the monitored probe intensities. Instead of the MM probes, the existing systematic biases are removed based on the observed intensities of the background probe probes (BGP) which are designed by Affymetrix. The BGPs are composed of the genomic and antigenomic probes. The genomic BGPs are selected from a research prototype human exon array design based on NCBI build 31. The antigenomic background probe sequences are derived based on reference sequences that are not found in the human (NCBI build 34), mouse (NCBI build 32), or rat (HGSC build 3.1) genomes. Multiple probes per exon enable “exon-level” analysis provide a basis for distinguishing between different isoforms of a gene. This exon-level analysis on a whole-genome scale opens the door to detecting specific alterations in exon usage that may play a central role in disease mechanism and etiology.

In some embodiments, each MAP 52 comprises the cellular constituent abundance values from a microRNA microarray. MicroRNAs (miRNAs) are a class of non-coding RNA genes whose final product is, for example, a 22 nucleotide functional RNA molecule. MicroRNAs play roles in the regulation of target genes by binding to complementary regions of messenger transcripts to repress their translation or regulate degradation. MicroRNAs have been implicated in cellular roles as diverse as developmental timing in worms, cell death and fat metabolism in flies, haematopoiesis in mammals, and leaf development and floral patterning in plants. MicroRNAs may play roles in human cancers. Examples of exon microarrays include, but are not limited to, the Agilent Human miRNA Microarray kit which contains probes for 470 human and 64 human viral microRNAs from the Sanger database v9.1.

In some embodiments, a MAP 52 comprises protein abundance or protein modification measurements that are made using a protein chip assay (e.g., The PROTEINCHIP® Biomarker System, Ciphergen, Fremont, Calif.). See also, for example, Lin, 2004, Modern Pathology, 1-9; Li, 2004, Journal of Urology 171, 1782-1787; Wadsworth, 2004, Clinical Cancer Research 10, 1625-1632; Prieto, 2003, Journal of Liquid Chromatography & Related Technologies 26, 2315-2328; Coombes, 2003, Clinical Chemistry 49, 1615-1623; Mian, 2003, Proteomics 3, 1725-1737; Lehre et al., 2003, BJU International 92, 223-225; and Diamond, 2003, Journal of the American Society for Mass Spectrometry 14, 760-765, each of which is hereby incorporated by reference herein in its entirety. Protein chip assays (protein microarrays) are commercially available. For example, Ciphergen (Fremont, Calif.) markets the PROTEINCHIP® System Series 4000 for quantifying proteins in a sample. Furthermore, Sigma-Aldrich (Saint Lewis, Mo.) sells a number of protein microarrays including the PANORAMA™ Human Cancer v1 Protein Array, the PANORAMA™ Human Kinase v1 Protein Array, the PANORAMA™ Signal Transduction Functional Protein Array, the PANORAMA™ AB Microarray—Cell Signaling Kit, the PANORAMA™ AB Microarray—MAPK and PKC Pathways kit, the PANORAMA™ AB Microarray—Gene Regulation I Kit, and the PANORAMA™ AB Microarray—p53 pathways kit. Further, TeleChem International, Inc. (Sunnyvale, Calif.) markets a Colorimetric Protein Microarray Platform that can perform a variety of micro multiplexed protein microarray assays including microarray based multiplex ELISA assays. See also, MacBeath and Schreiber, 2000, “Printing Proteins as Microarrays for High-Throughput Function Determination,” Science 289, 1760-1763, which is hereby incorporated by reference herein in its entirety.

In some embodiments, a MAP 52 comprises the cellular constituent abundance values measured using any of the techniques or microarrays disclosed in Section 5.5, below. In some embodiments, a MAP 52 comprises a plurality of cellular constituent abundance measurements 58 that consists of cellular constituent abundance measurements for between 10 oligonucleotides and 5×10⁶ oligonucleotides. In some embodiments, a MAP 52 comprises a plurality of cellular constituent abundance measurements that consists of cellular constituent abundance measurements for between 100 oligonucleotides and 1×10⁸ oligonucleotides, between 500 oligonucleotides and 1×10⁷ oligonucleotides, between 1000 oligonucleotides and 1×10⁶ oligonucleotides, or between 2000 oligonucleotides and 1×10⁵ oligonucleotides. In some embodiments, a MAP 52 comprises a plurality of cellular constituent abundance measurements that consists of cellular constituent abundance measurements for more than 100, more than 1000, more than 5000, more than 10,000, more than 15,000, more than 20,000, more than 25,000, or more than 30,000 oligonucleotides. In some embodiments, each MAP 52 comprises a plurality of cellular constituent abundance measurements that consists of cellular constituent abundance measurements for less than 1×10⁷, less than 1×10⁶, less than 1×10⁵, or less than 1×10⁴ oligonucleotides.

In some embodiments, a MAP 52 comprises a plurality of cellular constituent abundance measurements that consists of cellular constituent abundance measurements for between 5 mRNA and 50,000 mRNA. In some embodiments, a MAP 52 comprises a plurality of cellular constituent abundance measurements that consists of cellular constituent abundance measurements for between 500 mRNA and 100,000 mRNA, between 2000 mRNA and 80,000 mRNA, or between 5000 mRNA and 40,000 mRNA. In some embodiments, each MAP 52 comprises a plurality of cellular constituent abundance measurements that consists of cellular constituent abundance measurements for more than 100 mRNA, more than 500 mRNA, more than 1000 mRNA, more than 2000 mRNA, more than 5000 mRNA, more than 10,000 mRNA, or more than 20,000 mRNA. In some embodiments, each MAP 52 comprises a plurality of cellular constituent abundance measurements that consists of cellular constituent abundance measurements for less than 100,000 mRNA, less than 50,000 mRNA, less than 25,000 mRNA, less than 10,000 mRNA, less than 5000 mRNA, or less than 1,000 mRNA.

In some embodiments, each microarray 52 comprises a plurality of cellular constituent abundance measurements that consists of cellular constituent abundance measurements for between 50 proteins and 200,000 proteins. In some embodiments, each MAP 52 comprises a plurality of cellular constituent abundance measurements that consists of cellular constituent abundance measurements for between 25 proteins and 500,000 proteins, between 50 proteins and 400,000 proteins, or between 1000 proteins and 100,000 proteins. In some embodiments, each MAP 52 comprises a plurality of cellular constituent abundance measurements that consists of cellular constituent abundance measurements for more than 100 proteins, more than 500 proteins, more than 1000 proteins, more than 2000 proteins, more than 5000 proteins, more than 10,000 proteins, or more than 20,000 proteins. In some embodiments, each MAP 52 comprises a plurality of cellular constituent abundance measurements that consists of cellular constituent abundance measurements for less than 500,000 proteins, less than 250,000 proteins, less than 50,000 proteins, less than 10,000 proteins, less than 5000 proteins, or less than 1,000 proteins.

In some embodiments, the MAP data of step 204 is stored in a MAP data store 50. In some embodiments, the MAP data store 50 comprises data from a plurality of MAP 52 run in step 204, where the plurality of MAP 52 consists of between 50 MAPs 52 and 100,000 MAPs 52. In some embodiments, the MAP data store 50 comprises data from a plurality of MAPs 52 run in step 204, where the plurality of MAPs 52 consists of between 500 and 50,000 MAPs 52. In some embodiments, the MAP data store 50 comprises data from a plurality of MAPs 52 run in step 204, where the plurality of MAPs 52 consists of between 100 MAPs 52 and 35,000 MAPs 52. In some embodiments, the MAP data store 50 comprises data from a plurality of MAPs 52 run in step 204, where the plurality of MAPs 52 consists of between 50 MAPs 52 and 20,000 MAPs 52.

In some embodiments, a MAP 52 is measured from a microarray comprising probes arranged with a density of 100 different probes per 1 cm² or higher. In some embodiments, a MAP 52 is measured from a microarray comprising probes arranged with a density of at least 2,500 different probes per 1 cm², at least 5,000 different probes per 1 cm², or at least 10,000 different probes per 1 cm². In some embodiments, a microarray profile 52 is measured from a microarray comprising at least 10,000 different probes, at least 20,000 different probes, at least 30,000 different probes, at least 40,000 different probes, at least 100,000 different probes, at least 200,000 different probes, at least 300,000 different probes, at least 400,000 different probes, or at least 500,000 different probes.

As used herein, a microarray (which is used to obtain the data for a MAP 52 in some embodiments) is an array of positionally-addressable binding (e.g., hybridization) sites on a support. In some embodiments, the sites are for binding to many of the nucleotide sequences encoded by the genome of a cell or organism, most or almost all of the transcripts of genes or to transcripts of more than half of the genes having an open reading frame in the genome. In some embodiments, each of such binding sites consists of polynucleotide probes bound to the predetermined region on the support. Microarrays can be made in a number of ways, of which several are described in Section 5.5. However produced, preferably microarrays share certain characteristics. The arrays are reproducible, allowing multiple copies of a given array to be produced and easily compared with each other. In some embodiments, the microarrays are made from materials that are stable under binding (e.g., nucleic acid hybridization) conditions. Microarrays are preferably small, e.g., between 1 cm² and 25 cm², preferably 1 to 3 cm². However, both larger and smaller arrays (e.g., nanoarrays) are also contemplated and may be preferable, e.g., for simultaneously evaluating a very large number or very small number of different probes.

Step 206. In step 206, gene expression profiling is performed with each compound from a reserve library of compounds, such as drugs that have been approved by the FDA regardless of the performance of such drugs in step 202 and regardless of whether such compounds were in fact tested in step 202. In some embodiments, all or a portion of the compounds in the reserve library of compounds are tested in step 202. In some embodiments, none of the compounds in the reserve library of compounds are tested in step 202. Such compounds are referred to herein as validated compounds because such compounds have been approved by a regulatory agency. This does not mean, nor is there any requirement, that such compounds have demonstrated activity against the condition or disease of interest in this screening method. For each respective compound in the reserve library of compounds, the respective compound is exposed to one or more cell lines and then cellular constituent abundance values for a plurality of cellular constituents in the one or more cell lines is measured using microarray profiles. In some embodiments, the reserve library of compounds initially contains compounds approved by the United States Food and Drug Administration (and/or some other governing authority that has the power to approve the use of drugs in a country) and is then extended to include additional compounds of known activity. Over time, these compounds are profiled to identify the specific pathways and targets they uniquely affect. In some embodiments, each of the compounds in the reserve library is exposed to two or more cell lines, three or more cell lines, five or more cell lines, or ten or more cell lines resulting in two or more MAPs 52, three or more MAPs 52, five or more MAPs 52, or ten or more MAPs 52.

Step 208. Performance of steps 204 and 206 results in the creation of a very large number of MAPs 52 (e.g., 100 or more MAPs 52, 1000 or more MAPs 52, 10,000 or more MAPs 52, or 100,000 or more MAPs 52). In step 208, the MAPs 52 are used to construct a cellular network for a specific cellular phenotype under study. For instance, in some embodiments the cellular phenotype is a disease.

A cellular network comprises the identity of the proteins in the cell lines that have been tested (e.g., nodes) and the set of molecular interactions between these proteins (e.g, edges). In some embodiments, each edge represents a protein-protein interaction, a protein-DNA interaction or a transcription factor modulatory interaction (TFMI). In some embodiments, each edge is either directed or undirected. In some embodiments, a directed edge represents an interaction for which there is a molecule that is an activator or a modulator and a molecule that is regulated target of the modulator (e.g., a protein-DNA interaction or a TFMI). In some embodiments, an undirected edge represents proteins that bind to each other to form a complex (e.g., a protein-protein interaction or a transcription factor—transcription factor interaction).

The cellular phenotype under study is a disease and the cell lines under study in steps 202 through 206 are chosen so that they either best represent the disease or best represent control cells that do not exhibit the disease. In typical embodiments, cell lines are chosen for steps 202 through 206 to ensure that the compounds identified in the assays of steps 202 through 206 are both effective against the disease of interest and are selective for the disease of interest. For example, in some embodiments, the disease under study is breast cancer. In this case, one or more breast cancer cell types are chosen for use in the screens that are performed in steps 202 through 206. Because selective compounds are desired, the one or more cell types will typically include cell types that represent the disease of interest as well as cell types that, while closely related to the cell types of interest, are not themselves of interest. For example, consider the case of breast cancer where there is (i) basal breast cancer which is a very aggressive form of cancer for which there is almost no cure and (ii) normal breast cancer carcinomas for which there treatments that have some degree of success. If the desire is to find compounds that are very active against basal breast cancer but not the normal breast cancers, than the cellular network that is constructed using the assay results from steps 202 through 206 is built for basal breast cancer, using MAP data obtained from tissue samples that are representative of the basal breast cancer phenotype. Such a process allows for increased specificity on the phenotypic target. A specific disease, rather than a broad class of diseases, can be targeted. Thus, in some embodiments, what is desired are compounds that are very specific in, for example, ninety-nine percent of the subjects in a subpopulation that represents only, for example, twenty percent of the overall population rather than a compound that is applicable to a larger percent of the population but that is not specific to a the disease of interest but rather is applicable to a broad class of diseases.

The assays presented herein provide methods for performing personalized medicine where the cell lines are chosen from specific subpopulations. For example, consider the case of non-Hodgkins lymphoma which is potentially thirty different diseases. So, if a subject has non-Hodgkins lymphoma, they may have any one of thirty different subtypes. Because of this, an attempt to devise a cure that will cure all of these subtypes will likely result in a compound that is toxic due to a lack of specificity. Thus, in one embodiment, the goal is to work with individual sub-types of a disease (e.g., individual subtypes of non-Hodgkins lymphoma such as the ABC and GCB subtypes of Diffuse Large B Cell Lymphoma) that are very similar and homogenous at the molecular level. In the case of non-Hodgkins lymphoma, two subtypes of this disease are ABC and GCB Diffuse Large B Cell Lymphoma (DLBCL) and they have very different treatment efficacies. If ABC is of interest, the goal of step 202 is to identify compounds that have very high efficacy for ABC DLBCL but are not active or are less active in GCB DLBCL lymphoma. The goals of steps 204 and 206, then, are to screen the compounds identified in step 202 in the ABC non-Hodgkins cell type.

In order to build the cellular network, the MAP 52 data of steps 204 and 206 are subjected to analysis in order to identify cellular constituent interactions including, but not limited to, transcription factor interactions, protein-protein interactions whereby proteins for complexes, and modulators of proteins (e.g., modulators of transcription factors), and optionally microRNA interactions. In some embodiments this analysis includes an ARACNe (algorithm for the reconstruction of accurate cellular networks) analysis. See, for example, Margolin et al., 2006, Nature Protocols 1, 663-672; Basso et al., 2005, Nature Genetics 37, 382-390; Palomero, 2006, and Proceedings National Academy of Sciences 103, 18261-18266, each of which is hereby incorporated by reference herein in its entirety. ARACNe is designed to identify protein-DNA interactions (e.g., the target genes of a transcriptional factor). ARACNe uses the MAP 52 data from steps 204 and 206 to infer the transcriptional targets of any expressed transcription factor in the cell. ARACNe first identifies statistically significant gene-gene coregulation by an information theoretic measure such as mutual information using the cellular constituent abundance values for cellular constituents in the microarrray profiles measured in steps 204 and 206. It then eliminates indirect relationships, in which two cellular constituents are coregulated through one or more intermediaries, by making use of the data processing inequality (DPI). Therefore, relationships identified by ARACNe have a high probability of representing either direct regulatory interactions or interactions mediated by post-transcriptional modifiers that are undetectable from gene-expression profiles. See Basso et al., 2005, Nature Genetics 37, 382-390, which is hereby incorporated by reference herein in its entirety. In some embodiments this analysis comprises inferring one or more transcriptional targets of each of one or more expressed transcription factors, where the inferring comprises identifying a gene-gene coregulation between a first cellular constituent in the plurality of cellular constituents measured in the MAP 52 data of steps 204 and 206 that is a transcriptional target and a second cellular constituent in the plurality of cellular constituents measured in the MAP 52 data of steps 204 and 206 that is a transcription factor from the information theoretic measure I(X; Y) of the set of cellular constituent abundance values X for the first cellular constituent x and the set of cellular constituent abundance values Y for the second cellular constituent y. Here, X is the set of cellular constituent abundance values {x₁, . . . xₙ} measured from the plurality of MAPs 52, where each xᵢ in X is a measure of the cellular constituent abundance value of the first cellular constituent x in a different MAP 52 in the plurality of MAPs. Thus, X is a measure of x across the plurality of MAPs. Further, Y is the set of cellular constituent abundance values {y₁, . . . , yₙ} measured from the plurality of MAPs for y, where each yᵢ in Y is a measure of the cellular constituent abundance value of the second cellular constituent y in a different MAP 52 in the plurality of MAPs. Thus, Y is a measure of the cellular constituent abundance value of y across the plurality of MAPs. As used herein, the term “across” means “in each of.” For example, if there are ten MAPs in a plurality of maps, the cellular constituent abundance value of y across the plurality of MAPs means the cellular constituent abundance value of y in each MAP in the plurality of MAPs. In some embodiments, what is being compared is variance of X and variance of Y over the set of MAPs collectively measured in steps 204 and 206. In some embodiments, the information theoretic measure is the mutual information I(X; Y) of X and Y. Nonlimiting examples of transcription factors is provided in Section 5.8.

In one implementation, an information theoretic measure of X and Y is determined by treating X and Y as vectors and computing a similarity metric between the two vectors (X and Y) using mutual information, a correlation, a T-test, a Chi² test, or some other parametric or nonparametric means. In some embodiments, an information theoretic measure of X and Y is a measure of similarity such as any of the sixty-seven measures of similarity described in McGill, “An Evaluation of Factors Affecting Document Ranking by Information Retrieval Systems,” Project report, Syracuse University School of Information Studies, which is hereby incorporated by reference herein in its entirety. In some embodiments, each value x in X and each value y in Y is not weighted. In some embodiments, each value x in X and each value y in Y is weighted by a method disclosed in McGill, “An Evaluation of Factors Affecting Document Ranking by Information Retrieval Systems,” Project report, Syracuse University School of Information Studies, which is hereby incorporated by reference herein in its entirety.

ARACNe, which is based on a mutual information analysis, as well as methods based on ARACNe that use an information theoretic measure other than mutual information, are not designed to detect transcriptional interactions in a cell that are modulated by a variety of mechanisms that prevent their representation as pure pairwise interactions between a transcription factor and the one or more targets of the transcription factor. Such interactions include, but are not limited to, transcription factor activation by phosphorylation and acetylation, formation of active complexes with one or more cofactors, and mRNA/protein degradation and stabilization processes. Thus, in some embodiments, the MAPs in steps 204 and 206 are subjected to additional analysis to uncover these ternary interactions. In some embodiments, this additional analysis is a MINDy analysis or an analysis that is similar to MINDy but uses an information theoretic measure other than mutual information. MINDy is designed to identify transcription factor modulatory interactions (TFMI). See, for example, Wang et al., 2006, “Genome-wide discovery of modulators of transcriptional interactions in human B lymphocytes,” RECOMB, Lecture Notes in Computer Science, 348-362, which is hereby incorporated by reference herein in its entirety. MINDy predicts post-translational modulators of transcription factor activity. Specifically, druggable targets capable of activating, or suppressing specific transcriptional programs are identified by a MINDy analysis of the data from steps 204 and 206. Like ARACNe, MINDy makes use of mutual information to determine statistical significance between the measured abundance values for the cellular constituents measured in steps 204 and 206. However, MINDy focuses on transcription factors by determining whether the ability of a transcription factor g_{TF} to regulate a target cellular constituent gₜ is modulated by a third cellular constituent gₘ. Thus, MINDy is designed to identify ternary interactions. In some embodiments, given the MAP dataset with N cellular constituents (the MAPs measured in steps 204 and 206) and an a-priori selected transcription factor g_{TF} (which is one of the cellular constituents in the plurality of cellular constituents whose abundance value is measured in the MAPs of steps 204 and 206) an initial pool of candidate modulators gₘ is selected from the N genes according to two criteria: (a) each gₘ has sufficient expression range in the datasets measured in steps 204 and 206 to determine statistical dependencies, and (b) cellular constituents that are not statistically independent of g_{TF} (e.g., based on mutual information analysis) are excluded. Each candidate modulator gₘ is a cellular constituent in the plurality of cellular constituents whose abundance value is measured in the MAPs of steps 204 and 206. Each candidate modulator gₘ is used to partition the MAPs measured in steps 204 and 206 into two equal-sized, non-overlapping subsets, Lₘ⁺ and Lₘ⁻, in which gₘ is respectively at its highest (gₘ⁺) and lowest (gₘ⁻) abundances in the plurality of MAPs tested in previous steps. For example, in some embodiments Lₘ⁺ are those MAPs in which gₘ abundance is in the top fifty percentile or more, the top forty percentile or more, the top thirty percentile or more, the top twenty percentile or more, or the top ten percentile or more relative to the entire panel of MAPs measured in the combined steps 204 and 206. In some embodiments Lₘ⁻ are those MAPs in which gₘ abundance is in the bottom fifty percentile or less, the bottom forty percentile or less, the bottom thirty percentile or less, the bottom twenty percentile or less, or the bottom ten percentile or less relative to the entire panel of MAPs measured in the combined steps 204 and 206. Then, the conditional information theoretic measure I^{±}=(g_{TF},gₜ|gₘ^{±}) is computed. In some embodiments this conditional mutual information takes the form: ΔI(g_{TF},gₜ|gₘ) where

ΔI(g_{TF},gₜ|gₘ)=|I(g_{TF},gₜ|gₘ⁺)−(g_{TF},gₜ|gₘ⁻)|

and where
I(g_{TF},gₜ|gₘ⁺) is an information theoretic measure (e.g. mutual information) of the relationship between the abundance value of the transcription factor g_{TF} and the abundance value of the target g_{T} across Lₘ⁺, given the abundance value of the post-translational modulator of transcription factor activity gₘ across Lₘ⁺; and

I(g_{TF},gₜ|gₘ⁻) is an information theoretic measure of the relationship between the abundance value of the transcription factor g_{TF} and the abundance value of the target g_{T} across Lₘ⁻, given the abundance value of the post-translational modulator of transcription factor activity gₘ across Lₘ⁻. See Wang et al, 2006, “Genome-wide discovery of modulators of transcriptional interactions in human B lymphocytes,” RECOMB, Lecture Notes in Computer Science, 348-362, which is hereby incorporated by reference herein in its entirety. In this way, cellular constituents gₘ that modulate the ability for a transcription factor to regulation a target gₜ are identified.

In some embodiments, the information theoretic measure used in the computation of I(g_{TF},gₜ|gₘ⁺) and I(g_{TF},gₜ|gₘ⁻) is mutual information, a correlation, a T-test, a Chi² test, or some other parametric or nonparametric means. In some embodiments, an information theoretic measure used here is a measure of similarity such as any of the sixty-seven measures of similarity described in McGill, “An Evaluation of Factors Affecting Document Ranking by Information Retrieval Systems,” Project report, Syracuse University School of Information Studies, which is hereby incorporated by reference herein in its entirety. In some embodiments, g_{TF}, gₜ, gₘ⁺, and gₘ⁻ are unweighted for purposes of computing the information theoretic measure. In some embodiments, g_{TF}, gₜ, gₘ⁺, and gₘ⁻ are weighted for purposes of computing the information theoretic measure, using, for example any of the weighting methods set forth in McGill, “An Evaluation of Factors Affecting Document Ranking by Information Retrieval Systems,” Project report, Syracuse University School of Information Studies, which is hereby incorporated by reference herein in its entirety.

Step 210. The results from ARACNe and MINDY respectively provide numerous protein-DNA interactions and transcription factor modulatory interactions. In some embodiments, the ARACNe and MINDY data is assembled along with other data into an integrated mixed-interaction network using a Bayesian evidence integration framework such as the framework disclosed in Lefebvre et al., 2006, “A context-specific network of protein-DNA and protein-protein interactions reveals new regulatory motifs in human B cells,” Recomb Satellite on Systems Biology, San Diego, Calif.; as well as Mani et al., 2008, Molecular Systems Biology 4, 169, each of which is hereby incorporated by reference herein in its entirety. As used herein, the term interaction network is any network of molecular interactions relevant to the phenotype of interest. In some embodiments, the interaction network is a list of transcription factors and their targets. In some embodiments, the interaction network further comprises one or more transcription factor modulatory interactions. In some embodiments, the interaction network for a phenotype of interest is already known (e.g., from the literature). In such embodiments it is not necessary to perform steps 208 or 210. In some embodiments, a interaction network is any molecular interaction network built by observing correlations or some other information theoretic measure between cellular constituent abundances in cell samples upon exposure of such cell samples to various compounds or other perturbations (e.g., exposure to environmental factors such as temperature, culture media temperature) or genetic manipulations of such cell samples (e.g., point mutations). Examples of the construction of such molecular interaction networks provided herein are merely exemplary and any of several other techniques not disclosed herein can be used to construct such molecular interaction networks.

In some embodiments, the interaction network comprises of protein-protein (PP) and protein-DNA (PD) interactions in the context of the phenotype under study. This includes same-complex protein interactions and transient ones, such as those supporting signaling pathways. In some embodiments, the interaction network further comprises of the post-translational interactions predicted by the MINDy algorithm. These interactions include those cases where the ability of a transcription factor (TF) to regulate its target(s) (T) is modulated by a third protein (M) (e.g., an activating kinase). In some embodiments, the interaction network is generated by applying a Naïve Bayes classification algorithm using evidences from a variety of sources and gold-standard positive (GSP) and gold-standard-negative GSN) sets, to integrate the experimental and computational evidence. In some embodiments, the gold-standard evidence is drawn from several sources, including literature mining from GeneWays (Rzhetsky et al, 2004, J Biomed Inform. 37, 43-53, which is hereby incorporated by reference herein in its entirety), transcription factor-binding motif enrichment, orthologous interactions from model organisms, and reverse engineering algorithms, including ARACNe and MINDy for regulatory and post-translational interactions, respectively. A likelihood ratio (LR) for each evidence source is generated using the positive and negative gold-standard sets. Individual LRs are then combined into a global LR for each interaction. A threshold corresponding to a posterior probability greater than a predetermined threshold (e.g. P≧0.5) is used to qualify interactions as present or absent. In some embodiments, the additional sources of data that are integrated into the network using the Bayes classifier along with the protein-DNA interactions identified by ARACNe are protein-protein interaction data from sources such as the Gene Ontology biological process annotations (Ashburner et al., 2000, Nature Genetics 25, 25-29, which is hereby incorporated by reference herein in its entirety), data obtain from the GeneWays literature datamining algorithm (Rzhetsky et al., 2004, J Biomed Inform. 37, 43-53, which is hereby incorporated by reference herein in its entirety), and/or other sources. In some embodiments, additional protein-nucleic interaction data sources of data (in addition to or instead of the protein-nucleic interaction data provided by ARACNe) are integrated to form the interaction network using the Bayes classifier. Such additional protein-nucleic interaction data can be obtained from sources such as the GeneWays literature datamining algorithm.

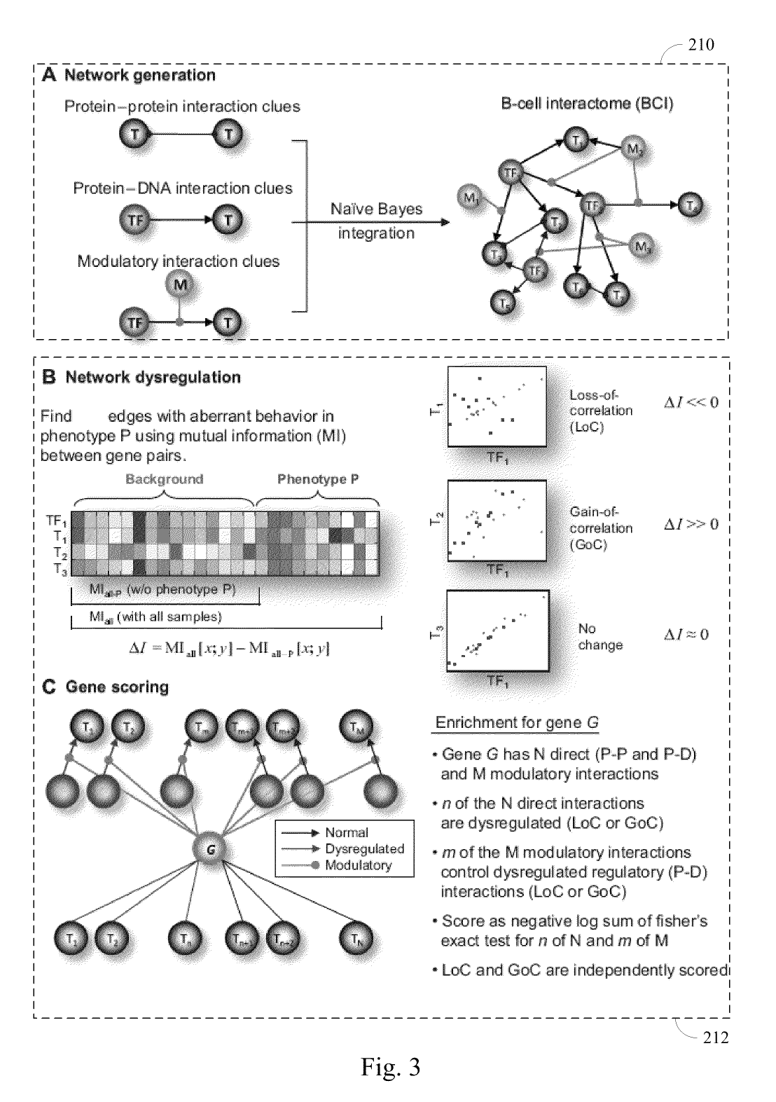

The Bayesian evidence integration framework allows for the integration of different sources of protein-protein interactions and protein-DNA interactions into a final set of interactions each with a posterior probability of greater than a threshold percent (e.g., fifty percent) of being a true interaction thereby forming the interaction network. Step 210 is illustrated in panel A of FIG. 3. In the graph shown in panel A of FIG. 3, directed edges indicate protein-DNA interactions and undirected edges indicate protein-protein (P-P) interactions or modulation events.

Step 212. In step 212, an interaction set enrichment analysis is performed to determine the drug activity profile of each of the compounds tested in steps 204 and 206 against the interaction network constructed in steps 208 and 210. Specifically, for a given compound, the edges in the interaction network that show aberrant behavior after treatment with the compound are identified using mutual information between cellular constituent pairs. Panel B of FIG. 3 illustrates this step.

In some embodiments, in steps 204 and 206, cell lines both representative of the phenotype under study (e.g., a particular disease or more preferably, a particular disease subtype) and cell lines not representative of the phenotype under study are each exposed to the compound under study before performing MAP analysis and thereby measuring a microarray profile from each cell line exposed to the compound. Edges (interactions) between any pair of cellular constituents that are found in the resultant interaction network constructed in steps 208 and 210 that show aberrant behavior are then identified in step 212. There are at least two types of aberrant behavior possible for each edge: loss of correlation (LoC) between the two cellular constituents that the edge connects and gain of correlation (GoC) between the two cellular constituents that the edge connects. In some embodiments, the data from steps 204 and 206 can be used to perform the interaction set enrichment analysis and in such embodiments step 212 advantageously does not require any wet lab experimentation that has not already been done in previous steps.

In some embodiments, the test for aberrant behavior of an edge is determined based on the estimate of an information theoretic measure, such as mutual information, in the MAPs of the two cellular constituents that make up the edge in the interaction network. Mutual information is an information theoretic measure of statistical dependence, which is zero if and only if two variables are statistically independent. Mutual information can be calculated, for example, using a Gaussian kernel estimation. See, for example, Margolin et al., 2006, BMC Bioinformatics 7 (Suppl 1:) S7, which is hereby incorporated by reference herein in its entirety. In one such embodiment, an edge in the interaction network is tested to see whether mutual information increases (Loc) or decreases (GoC) when the samples corresponding to the specific phenotype are removed from the entire compendium of datasets measured in steps 204 and 206 (used to compute the background mutual information). A null distribution is computed to assess the statistical significance of mutual information changes as a function of the background mutual information and of the number of removed samples. In some embodiments, an edge in the interaction network between cellular constituents a and b is deemed to be affected in the phenotype P, if and only if the following information theoretic measure difference is statistically significant:

ΔI=I_{AH}[A;B]−I_{AH-P}[A;B]

where I_{AH}[A;B] is an information theoretic measure between cellular constituent abundance values A for the cellular constituent a where each Aᵢ in the set A={a₁, . . . , aₙ} is a cellular constituent abundance value for the cellular constituent a (e.g., transcription factor) in a microarray sample in the MAPs tested in steps 204 and 206 collectively, and each Bᵢ in the set B={b₁ . . . , bₙ} is a cellular constituent abundance value for the cellular constituent b (e.g., cellular constituent) in the plurality of MAPs. Further, I_{AH-P}[A;B] is an information theoretic measure between cellular constituent abundance values A for the cellular constituent a in each of the plurality of MAPs not taken from samples of cells exhibiting the phenotype of interest and cellular constituent abundance values B for the cellular constituent b in the plurality of MAPs not taken from samples of cells exhibiting the phenotype of interest.

In some embodiments, the information theoretic measure used to compute I_{AH}[A;B] and I_{AH-P}[A;B] is mutual information (MI) and the threshold that defines whether ΔI is statistically significant is calculated by sampling a subset of interactions across a predetermined number of equally sized MI bins (e.g., 100 bins) covering the full mutual information range in the interaction network. For each bin of interactions, sample sets of various sizes, representing the size of each phenotype group, are randomly removed from the dataset and the ΔI is calculated. A total of 10,000 values (or some other number of values) are computed for each bin and fit with a Gaussian distribution. In some embodiments, a Bonferroni corrected p-value of 0.05 is used to threshold a test for a given sample set size and original mutual information value. Note that the ΔI value will be negative in the LoC cases (as the mutual information increases after removal), and positive in the GoC cases (vice-versa). In some embodiments, all interactions that pass the threshold are labeled as −1 or 1 respectively. In some embodiments, some other information theoretic measure of statistical dependence is used to identify aberrant behavior of an edge such as correlations, a T-test, a Chi² test, some other parametric or nonparametric means, or any of the measures of similarity disclosed in McGill, “An Evaluation of Factors Affecting Document Ranking by Information Retrieval Systems,” Project report, Syracuse University School of Information Studies, which is hereby incorporated by reference herein in its entirety.

LoC interactions are interactions that show correlation in all cell lines except the cell lines representative of P, the phenotype under study. For example, consider panel B of FIG. 3 in which interactions between a transcription factor TF₁ and three targets of TF₁, T₁, T₂, and T₃, are listed. The abundance data from steps 204 and 206 provides abundance data for TF₁, T₁, T₂, and T₃ in each of several cell types including those not representative of the desired phenotype (background) and those with the desired phenotype (P). In the exemplary data, there is loss of correlation between T₁ and TF₁ as illustrated in the correlation chart between T₁ and TF₁ because there is a degree of correlation in the expression of T₁ and TF₁ in background cell lines, as determined by mutual information, but there is considerably less correlation in the expression of T₁ and TF₁ in cell lines that have phenotype P.

GoC interactions are interactions that show correlation in all cell lines representative of P but not in background cell lines. For example, consider panel B of FIG. 3 in which, in accordance with the exemplary data, there is gain of correlation between TF₁ and T₂ as illustrated in the correlation chart because there is a degree of correlation in the expression of TF₁ and T₂ in cell lines representative of the phenotype P, as determined by mutual information, but there is considerably less correlation in the expression of TF₁ and T₂ in background cell lines.

In some embodiments, in steps 204 and 206, cell lines representative of the phenotype under study (e.g., a particular disease or more preferably, a particular disease subtype) are exposed to the compound under study before performing MAP analysis. Furthermore, in some embodiments the same cell lines that are representative of the phenotype under study are not exposed to the compound under study before performing MAP analysis. Edges (interactions) between transcription factors TF (e.g., TF₁) and their targets (e.g., T₁, T₂, . . . , T_{N}) found in the interaction network constructed in steps 208 and 210 can then be analyzed for aberrant behavior between the cell lines exposed and not exposed to the compound. Here, loss of correlation (LoC) between the two cellular constituents that the edge connects are those interactions that show correlation in all cell lines not exposed to the compound but not in cell lines not exposed to the compound. Gain of correlation (GoC) between the two cellular constituents that the edge connects are those interactions that show correlation in all cell lines exposed to the compound but not in the cell lines that have not been exposed to the compound.

Of course, various combinations of the two embodiments given above, that is (i) comparison of cell types of phenotype P to cell types of background phenotype to identify dysregulated interactions (edges in the Interactome graph) where all cell types are exposed to compound of interest and (ii) comparison of cell types exposed to compound of interest to cell types not exposed to compound of interest to identify dysregulated interactions, can be used to identify the interactions that a given compound affects.

Once the dysregulated interactions in the interaction network have been determined for a given compound under study, these dsyregulated interactions are pooled together and a statistical enrichment is calculated which identifies cellular constituents having an unusually high number of dysregulated interactions in their neighborhood, when either direct or modulated interactions are considered. The list of cellular constituents that are significantly affected by a compound is termed the drug activity profile of the compound.

In some embodiments cellular constituents are scored by the enrichment of their direct network neighborhood in GoC/LoC interactions, using a Fisher’ exact test. Specifically, in such an approach for both LoC and GoC, two partial p-values are separately computed, based on the number of dysregulated interactions a cellular constituent is directly involved in or is modulating within its direct neighborhood. A global p-value is then computed as the product of all four partial p-values. More specifically, in some embodiments, enrichment for each cellular constituent is calculated using a set of hypergeometric tests. For the phenotype, all affected interactions are split into LoC or GoC categories. A p-value for each case is computed, based on the total interactions (N), the number of LoC or GoC interactions the cellular constituent is directly connected to (D), its natural connectivity in the interaction network (H), and the size of the overall LoC/GoC signature for that particular phenotype (S). As shown below, the p-value is equivalent to a Fisher Exact Test, and is computed for LoC and GoC cases separately.

$$p - value ( G ) = 1 - {\int }_{i = 1}^{D - 1} \frac{( \begin{matrix}H \\ i\end{matrix} ) ( \begin{matrix}N - H \\ S - i\end{matrix} )}{( \begin{matrix}N \\ S\end{matrix} )}$$

An additional set of p-values is computed based on modulatory interactions from each cellular constituent as well. As noted above, in some embodiments the predictions from the MINDy-type algorithm about three way interactions between a transcription factor, its target, and a third modulator cellular constituent are incorporated into the interaction network. Thus, an enrichment based on the number of interactions a constituent is predicted to modulate that fall into the LoC or GoC category is included in some embodiments. In total, these four p-values are combined in a negative log sum operation in order to invoke the simplifying assumption that LoC and GoC cases can be treated independently, as can direct effects and modulatory effects. Although this type of enrichment may bias the analysis against hubs, it can still identify those hubs when they are, in fact, related to the phenotype being analyzed. There are several alternative ways of computing a dysregulation score for cellular constituents. For instance, in some embodiments, the Gene Set Enrichment Analysis method can be used to compute such a score by considering the enrichment of the interactions supported by a cellular constituent against all interactions sorted from the one with highest LOC to the one with highest GOC. Furthermore, there are several alternatives to combine scores for different types of interactions and LOC/GOC, all of which are encompassed herein.

Those cellular constituents that are determined to be affected by a respective compound on a statistically significant basis (e.g. a p-value of 0.10 or less, 0.05 or less, or 0.005 or less) are deemed to comprise the drug activity profile of the compound. By performing the analysis described in this step for each of the compounds under study, a drug activity profile is defined for each of the compound under study.

Step 214. In step 214, the compounds that have been tested are filtered to form a filtered set of compound combinations. In some embodiments, a compound will be included one or more compound combinations in the filtered set of compound combinations if it satisfies any one of the following three criteria:

(i) the compound has demonstrated efficacy in step 202 (e.g., the compound causes a desired end-point phenotype such as cell death);

(ii) the compound has not demonstrated efficacy in step 202 but, from the drug activity profile of the compound from step 212 and the interaction network of step 210, it is seen that the compound hits one or more targets that are synergistic to the targets in the drug activity profile of at least one compound qualifying under criterion (i); or

(iii) the compound has been designed to specifically inhibit a target that has been computationally identified as being synergistic to the targets in the drug activity profile of at least one compound qualifying under criterion (i).

In some embodiments there exists a cellular constituent signature for the desired end-point phenotype. In some embodiments, the cellular constituent signature for the desired end-point phenotype is the difference in cellular constituent abundance between (i) a cell sample representative of the phenotype of interest but is not exhibiting the desired end-point phenotype (e.g., Diffuse Large B Cell Lymphoma, DLBCL that is alive) and (ii) a cell sample representative of the phenotype of interest but that also exhibits the desired end-point phenotype (e.g., DLBCL cells undergoing apoptosis). For example, consider the case in which there are a plurality of cellular constituents whose abundances are measured in (i) a first cell sample representative of the phenotype of interest (e.g., DLBCL that are not undergoing apotosis) and (ii) a second cell sample representative of the phenotype of interest but that also exhibit the desired end-point phenotype (e.g., DLBCL cells undergoing apoptosis). In this example, the cellular constituent signature for the desired end-point phenotype (apotosis) is the differential cellular constituent abundance of each cellular constituent between the first cell sample and the second cell sample.

In some embodiments in which the cellular constituent signature for the desired end-point phenotype is available, the filtering in step 214 comprises assigning a score to each of the candidate compounds. In some embodiments, the score for a given candidate compound is a similarity between (i) the differential cellular constituent abundances in the differential profile of the candidate compound as described above in conjunction with step 202 and (ii) the differential cellular constituent abundances in the cellular constituent signature of the desired end-point phenotype. In some embodiments, this measure of similarity is calculated by mutual information, a correlation, a T-test, a Chi² test, or some other parametric or nonparametric means. In some embodiments, the measure of similarity is any of the sixty-seven measures of similarity described in McGill, “An Evaluation of Factors Affecting Document Ranking by Information Retrieval Systems,” Project report, Syracuse University School of Information Studies, which is hereby incorporated by reference herein in its entirety.

In some embodiments in which multiple differential profiles for the candidate compound have been made as described above in conjunction with step 202, the score for the respective compound can be some mathematical combination of the similarity of the differential cellular constituent abundances in the cellular constituent signature of the desired end-point phenotype against each of the differential cellular constituents abundances in the differential profiles of the candidate compound produced for the candidate compound.

In some embodiments, once a score has been assigned to each of the candidate compounds as described above, a combination score is computed for each unique combination of candidate compounds. To compute the combination score, a measure of similarity between the differential cellular constituent abundances in the differential profiles of each of the compounds in the combination of compounds is determined. This measure of similarity can be calculated, for example, by mutual information, a correlation, a T-test, a Chi² test, or some other parametric or nonparametric means. In some embodiments, the measure of similarity is any of the sixty-seven measures of similarity described in McGill, “An Evaluation of Factors Affecting Document Ranking by Information Retrieval Systems,” Project report, Syracuse University School of Information Studies, which is hereby incorporated by reference herein in its entirety. For instance, if the desire is to obtain pairs of candidate compounds, a similarity score is computed for each unique pair of candidate compounds in the candidate set of compounds. In another example, if the desire is to obtain candidate compound triplets, a score is computed for each unique triplet of candidate compounds in the candidate set of compounds.

In some embodiments, the combinations of compounds are ranked by their combinations scores such that those compounds that have the least correlation between their differential profiles are ranked higher than those compounds that have the most correlation between their differential profiles. For example, consider the case in which a correlation coefficient is used to measure the similarity in the differential profile of a first and second compound, where a high correlation coefficient (close to 1) indicates that the differential abundances of the cellular constituents in the differential profile of the first compound and the differential profile of the second compound are similar. Compound pairs that receive a high correlation would be assigned a low combination score and ranked low on the ranked list of compounds. Further, compound pairs that receive a low correlation would be assigned a high combination score and ranked high on the ranked list of compounds. Of course, the concept of “low” and “high” as used herein for combination scores can be completely reversed and still be within the scope of the present invention provided that the compound combinations can be ranked in some manner as a function of their combination scores. From this ranked list, those compound combinations that have the least similar differential profiles are preferentially selected.

In some embodiments, each potential compound combination is selected based on two types of scores: (i) the individual similarity scores assigned to each compound based on their similarity to the cellular constituent signature of the desired end-point phenotype and (ii) and the combination score assigned to the potential compound combination. In the case where compound pairs are desired, each compound pair has (i) a score for a first compound against the cellular constituent signature of the desired end-point phenotype, (ii) a score for a second compound against the cellular constituent signature of the desired end-point phenotype, and (iii) a compound combination score. Those compound combinations that have relatively high individual similarity between the differential profiles of each compound in the combination against the cellular constituent signature for the desired end-point phenotype and relativity low compound combination scores are preferentially selected for the filter set of compound combinations in such embodiments.

In general, step 214 serves to identify each of the compounds suitable for further analysis. Combinations of compounds (e.g. combinations of two compounds, combinations of three compounds, combinations of four compounds) are of interest in some embodiments. Because combinations will be selected, in some embodiments the filtering imposed in this step does not impose the requirement that a respective compound have observed efficacy in step 202. In some embodiments, the filtering in this step uses a scoring function that seeks compounds that (i) form compound pairs or compound triplets (or some higher ordered compound combination) whose respective drug activity profiles involve genes that are in synergistic pathways rather than the same pathways and (ii) target specific pathways rather than being pleiotropic. In some embodiments, the scoring function in this step gives higher priority to compound combinations formed from compounds with well known toxicity profiles (e.g., compounds that have been approved for at least one medical indication by a drug approving agency such as the Food and Drug Administration in the United States or corresponding agencies in other countries). In some embodiments, the scoring function in this step gives higher priority to compound combinations where at least one of the compounds has a well known toxicity profile (e.g., has been approved for at least one medical indication by a drug approving agency such as the Food and Drug Administration in the United States or corresponding agencies in other countries).

As a result of the filtering in this step, compound combinations in the filtering set are depleted of compound combinations where each of the compounds in the combinations affect identical pathways that may not bypass the cell's redundancy mechanisms and are likely only to produce an additive effect, identical to using a larger dose of a single compound are eliminated in the filtering step. Eliminating such compound combinations will thereby enrich the filtered compound combination list for compounds combinations affecting independent pathways with the same end-point phenotype that produce a synergistic effect, thus allowing to more effectively defeat a target disease's defenses. Additionally, by selecting pathway and target combinations that are specific to the disease phenotype but not to the normal cells, toxicity and side effects are reduced. In some embodiments, at the end of this step, the original set 1,000,000³ potential compound combination is reduced to about 10,000 highest priority combinations based on the aforementioned steps.

Step 216. Among all the possible compound combinations from the filtered list of step 214, a top number of the most synergistic combinations (e.g. 1,000 to 10,000 combinations) are screened again using the phenotype of interest as well as background cell types in combination form using, for example, the experimental assay used in step 202, to assess their synergistic behavior in implementing the desired end-point phenotype. In these screens, the compounds are stratified against disease cells and normal background cells at various concentrations. For example, in one embodiment, a combination of two different compounds is tested, with each compound tested at three different concentrations for a total of nine different dosages. In another example, in one embodiment, a combination of three different compounds is tested, with each compound tested at three different concentrations for a total of 27 different dosages. Compound combinations achieving optimal selectivity in disease phenotype versus either other disease phenotypes or normal tissue are then screened in vivo for synergistic behavior. In some embodiments, at the end of this step, the original set 1,000,000³ potential compound combination is reduced to about 1 to 10 highest priority combinations based on the aforementioned steps that can be further prioritized for lead optimization, pre-clinical studies, and clinical studies.

The present invention provides variations of the above-identified method. In a first variation a interaction network is not used and thus steps 208, 210, and 212 are not performed. In this first variation a first plurality of cell-based assays are performed as described above in step 202. Each cell-based assay in the first plurality of cell-based assays comprises (i) exposing a different compound in a first plurality of compounds to a different sample of cells and (ii) measuring a phenotypic result of the different sample of cells upon exposure of the different compound, thereby obtaining a first plurality of phenotypic results as described in step 202. Typically, such exposing and measuring is done twice, where in one instance a first aliquot of cells is exposed to delivery medium without compound and in the other instance a second aliquot of cells is exposed to delivery medium that includes compound. Each phenotypic result in the first plurality of phenotypic results corresponds to a compound in the first plurality of compounds. From the first plurality of phenotypic results, a subset of compounds in the first plurality of compounds that cause a desired end-point phenotype are selected as described above in step 202.

Next, as described in step 204 above, for each respective compound in the subset of compounds, a MAP is measured using a different sample of cells that has been exposed to the respective compound thereby obtaining a first plurality of MAPs. Each MAP in the first plurality of MAPs comprises cellular constituent abundance values for a plurality of cellular constituents in a sample of cells that has been exposed to a compound in the subset of compounds. Further, MAPs may be obtained for compounds in a reference library of compounds as described above in step 206.

Then, rather than performing steps 208, 210, or 212, there is computed, for each respective compound in the subset of compounds, a compound similarity score between (i) a differential profile of the respective compound and (ii) a cellular constituent signature of the desired end-point phenotype, thereby calculating a plurality of compound similarity scores. The differential profile of the respective compound comprises differences in cellular constituent abundance values of each cellular constituent in a plurality of cellular constituents between (i) cells representative of the phenotype of interest (e.g., malignant state) that have not been exposed to the respective compound (e.g. cells that have only been exposed to delivery medium but not compound) and (ii) cells representative of the phenotype of interest (e.g., malignant state) that have been exposed to the respective compound (e.g., cells that have been exposed to delivery medium, such as DMSO, that includes compound). In some embodiments, the cellular constituent signature of the desired end-point phenotype comprises differences in cellular constituent abundance values of each cellular constituent in a plurality of cellular constituents between (i) a cell sample representative of a phenotype of interest (e.g., malignant state) that is not exhibiting a desired end-point phenotype and (ii) a cell sample representative of the phenotype of interest (e.g., malignant state) that is also exhibiting a desired end-point phenotype (e.g., undergoing apotosis). In some embodiments, the cellular constituent signature comprises differences in cellular constituent abundance values of each cellular constituent in a plurality of cellular constituents between (i) a cell sample or other biological sample representative of the phenotype of interest (e.g., malignant state) that has been exposed to delivery medium without compound for a time t₁ and (ii) a cell sample or other biological sample representative of the phenotype of interest (e.g., malignant state) that has been exposed to delivery medium with compound for a time t₁.

Next, a filter set of compound combinations comprising a plurality compound combinations is formed. Each compound combination is a combination of compounds in the subset of compounds, where a compound combination in the plurality of compound combinations is selected based on a combination of (i) a compound similarity score of each compound in the compound combination as determined above, and a difference in the differential profile of each compound, determined above, in the compound combination.

In some embodiments in accordance with this first variation, a compound in the first plurality of compounds is used in single cell-based assay in the first plurality of cell-based assays at a single concentration. In some embodiments in accordance with this first variation, a compound in the first plurality of compounds is used in a first cell-based assay in the first plurality of cell-based assays at a first concentration and is used in a second cell-based assay in the first plurality of cell-based assay at a second concentration. In some embodiments in accordance with this first variation, a compound in the first plurality of compounds is used in a plurality of cell-based assays in the first plurality of cell-based assays, where each cell-based assay in the plurality of cell-based assays in which the compound is used is at a same or different concentration. In some embodiments in accordance with this first variation, each respective compound in the first plurality of compounds is used in a plurality of cell-based assays in the first plurality of cell-based assays, where each cell-based assay in the plurality of cell-based assays in which a respective compound is used is at a same or different concentration. In some embodiments in accordance with this first variation, a compound in the first plurality of compounds is assayed in single cell-based assay in the first plurality of cell-based assays at a single time delay. In some embodiments in accordance with this first variation, a compound in the first plurality of compounds is assayed in a first cell-based assay in the first plurality of cell-based assays at a first time delay and is assayed in a second cell-based assay in the first plurality of cell-based assay at a second time delay. In some embodiments in accordance with this first variation, a compound in the first plurality of compounds is assayed in a plurality of cell-based assays in the first plurality of cell-based assays, where each cell-based assay in the plurality of cell-based assays in which the compound is used is assayed at a same or different time delay. In some embodiments in accordance with this first variation, each respective compound in the first plurality of compounds is assayed in a plurality of cell-based assays in the first plurality of cell-based assays, where each cell-based assay in the plurality of cell-based assays in which a respective compound is used is assayed after exposure of the cells sample to the compound for a same or different amount of time.

In some embodiments in accordance with this first variation, the measuring step further comprises measuring, for each respective compound in a plurality of validated compounds, a MAP using a different sample of cells or other biological sample that has been exposed to the respective compound in delivery medium (e.g., DMSO) thereby obtaining a second plurality of MAPs, each MAP in the second plurality of MAPs comprising cellular constituent abundance values for a plurality of cellular constituents in a sample of cells that has been exposed to a compound in the plurality of validated compounds. In some embodiments in accordance with this first variation, the performing further comprises performing a second plurality of cell-based assays, each cell-based assay in the second plurality of cell-based assays for a different compound in a plurality of validated compounds, each cell-based assay in the second plurality of cell-based assays comprising (i) exposing a different compound in the plurality of validated compounds to a different sample of cells, and (ii) measuring a phenotypic result of the different sample of cells upon exposure of the different compound, thereby obtaining a second plurality of phenotypic results, each phenotypic result in the second plurality of phenotypic results corresponding to a compound in the plurality of validated compounds. In some embodiments, a compound in the plurality of validated compounds is used in single cell-based assay in the second plurality of cell-based assays at a single concentration. In some embodiments, a compound in the plurality of validated compounds is used in a first cell-based assay in the second plurality of cell-based assays at a first concentration and is used in a second cell-based assay in the second plurality of cell-based assays at a second concentration. In some embodiments, a compound in the plurality of validated compounds is used in a plurality of cell-based assays in the second plurality of cell-based assays, where each cell-based assay in the plurality of cell-based assays in which the compound is used is at a same or different concentration.

In some embodiments in accordance with this first variation, each respective compound in the plurality of validated compounds is used in a plurality of cell-based assays in the second plurality of cell-based assays, where each cell-based assay in the plurality of cell-based assays in which a respective compound is used is at a same or different concentration. In some embodiments in accordance with this first variation, the method further comprises screening a subset of compound combinations in the filter set of compound combinations for their ability to implement the desired end-point phenotype. In some embodiments in accordance with this first variation, the method further comprises outputting the filter set of compound combinations in a format accessible to a user, to a computer readable storage medium, to a tangible computer readable storage medium, to a local or remote computer system, or to a display. As used herein, a local computer is a computer that is in the physical location where any of the steps described above in conjunction with FIG. 2 are carried out. As used herein, a remote computer is a computer that is not in the physical location where one or more of the steps described above in conjunction with FIG. 2 is carried out, but rather such remote computer is addressable over the Internet from the physical location where one or more of the steps described above in conjunction with FIG. 2 is carried out. In some embodiments in accordance with this first variation, the first plurality of compounds comprises one thousand compounds or more, ten thousand compounds or more, or one hundred thousand compounds or more.

In some embodiments in accordance with this first variation, the phenotype of interest is a disease, a cancer, bladder cancer, breast cancer, colorectal cancer, gastric cancer, germ cell cancer, kidney cancer, hepatocellular cancer, non-small cell lung cancer, non-Hodgkin's lymphoma, melanoma, ovarian cancer, pancreatic cancer, prostate cancer, soft tissue sarcoma, or thyroid cancer. In some embodiments in accordance with this first variation, the plurality of cellular constituents is between 5 mRNAs and 50,000 mRNAs and the cellular constituent abundance values are amounts of each mRNA. In some embodiments in accordance with this first variation, the plurality of cellular constituents is between 50 proteins and 200,000 proteins and the cellular constituent abundance values are amounts of each protein. In some embodiments in accordance with this first variation, each compound combination in the filter set of compound combinations consists of two different compounds in the subset of compounds. In some embodiments in accordance with this first variation, each compound combination in the filter set of compound combinations consists of three different compounds in the subset of compounds. In some embodiments in accordance with this first variation, the filter set of compound combinations comprises 10,000 or more compound combinations.

In some embodiments in accordance with this first variation, the filter set of compound combinations comprises 50,000 or more compound combinations. In some embodiments in accordance with this first variation, the screening step comprises performing a plurality of cell-based confirmation assays, each cell-based confirmation assay in the plurality of cell-based confirmation assays comprising (i) exposing a different compound combination in the filter set of compound combinations to a different sample of cells, and (ii) measuring a phenotypic result of the different sample of cells upon exposure of the different compound combination. In some embodiments in accordance with this first variation, the phenotypic result is cell death as a function of an amount of a compound in the different compound composition.

In a second variation of the method set forth in FIG. 2, a cellular constituent signature of the desired end-point phenotype is computed, where the cellular constituent signature of the desired end-point phenotype comprises differences in cellular constituent abundance values of each cellular constituent in a plurality of cellular constituents between (a) a cell sample exhibiting a phenotype of interest (e.g. cells representative of a physiologic or pathologic state) but that is not exhibiting a desired end-point phenotype and (b) a cell sample exhibiting a phenotype of interest but that is also exhibiting the desired end-point phenotype (e.g. cells representative of a physiologic or pathologic state and that are undergoing apotosis). For example, the phenotype of interest may be Diffuse Large B Cell Lymphoma (DLBCL) and the cell sample exhibiting the desired end-point phenotype may be that of DLBCL cells undergoing apoptosis. Using the cellular constituent signature of the desired end-point phenotype as well as the interaction network, a plurality of transcription factors that can implement the desired end-point phenotype is determined. The interaction network may be obtained from the literature or may be obtained using the techniques disclosed in step 208 (e.g., an ARACNe analysis). In this second variation of the method set forth in FIG. 2, the drug activity profile, for each respective compound in the subset of compounds, indicates whether the respective compound affects an abundance of one or more transcription factors in the plurality of transcription factors, as determined by the interaction network and a differential profile of the respective compound. Here, the differential profile of the respective compound comprises differences in cellular constituent abundance values of each cellular constituent in a plurality of cellular constituents between (i) a first aliquot of cells or other biological sample that have not been exposed to the respective compound (e.g., has not been exposed to anything or has just been exposed to a compound delivery vehicle that does not include the compound) and (ii) a second aliquot of cells or other biological sample that have been exposed to the respective compound. Typically, the first and second aliquot of cells or other biological sample exhibits the phenotype of interest (e.g., DLBCL) prior to exposure. In this second variation of the method set forth in FIG. 2, the forming step 214 comprises selecting a compound combination for the filter set of compound combinations based on a combination of (i) a drug activity profile of each compound in the compound combination, and (ii) a difference in the differential profile of each compound in the compound combination. What is desired are compound combinations in which the compounds have a drug activity profiles that show an effect on identified transcription profiles but where the compounds combinations have different differential profiles from each other. In this way, such compounds in a given compound combination are likely to affect the transcription factors that implement the desired end-point phenotype but do so in synergistic ways because they affect different cellular constituents in the plurality of cellular constituents.

In a third variation of the method set forth in FIG. 2, a cellular constituent signature of the desired end-point phenotype is computed, where the cellular constituent signature of the phenotype of interest comprises differences in cellular constituent abundance values of each cellular constituent in a plurality of cellular constituents between (a) a cell sample exhibiting a phenotype of interest but that is not exhibiting a desired end-point phenotype and (b) a cell sample that is exhibiting a phenotype of interest and that is also exhibiting a desired end-point phenotype. For example, the phenotype of interest may be a Diffuse Large B Cell Lymphoma (DLBCL) and (a) the cell sample exhibiting the phenotype of interest but is not exhibiting a desired end-point phenotype is live DLBCL cells whereas (b) the cell sample that is exhibiting the phenotype of interest and that is also exhibiting the desired end-point phenotype is DLBCL cells undergoing apoptosis. Using the cellular constituent signature of the desired end-point phenotype as well as the interaction network, a plurality of post-translational modulators of transcription factor activity that implement the desired end-point phenotype is determined. The interaction network may be obtained from the literature or may be obtained using the techniques disclosed in step 208 (e.g., a MINDy analysis). In this third variation of the method set for in FIG. 2, the drug activity profile, for each respective compound in the subset of compounds, indicates whether the respective compound affects the abundance of one or more post-translational modulators of transcription factor activity in the plurality of post-translational modulators of transcription factor activity as determined by the interaction network and a differential profile of the respective compound. Here, the differential profile of the respective compound comprises differences in cellular constituent abundance values of each cellular constituent in a plurality of cellular constituents between (i) a first aliquot of cells or other biological specimen exhibiting the phenotype of interest that have not been exposed to the respective compound (e.g., are not exposed to anything or have been exposed to a compound delivery medium that does not include compound) and (ii) a second aliquot of cells or other biological specimen exhibiting the phenotype of interest prior to exposure that have been exposed to the respective compound for a period of time. In this third variation of the method set forth in FIG. 2, the forming step 214 comprises selecting a compound combination for the filter set of compound combinations based on a combination of (i) a drug activity profile of each compound in the compound combination, and (ii) a difference in the differential profile of each compound in the compound combination. What is desired are compound combinations in which the compounds have a drug activity profiles that show an effect on the identified post-translational modulators of transcription factor activity but where the compounds combinations have distinct activity profiles from each other. In this way, such compounds in a given compound combination are likely to affect the plurality of post-translational modulators of transcription factor activity, but do so in synergistic ways because they affect different cellular constituents in the plurality of cellular constituents.

### 5.2 Exemplary Cell Types

Exemplary cell types that may be tested in steps 202, 204, 206, and 216 include, but are not limited to, keratinizing epithelial cells such as epidermal keratinocytes (differentiating epidermal cells), epidermal basal cells (stem cells), keratinocytes of fingernails and toenails, nail bed basal cells (stem cells), medullary hair shaft cells, cortical hair shaft cells, cuticular hair shaft cells, cuticular hair root sheath cells, hair root sheath cells of Huxley's layer, hair root sheath cell of Henle's layer, external hair root sheath cells, hair matrix cells (stem cells).

Exemplary cell types further include, but are not limited to, wet stratified barrier epithelial cells such as surface epithelial cells of stratified squamous epithelium of cornea, tongue, oral cavity, esophagus, anal canal, distal urethra and vagina, basal cells (stem cell) of epithelia of cornea, tongue, oral cavity, esophagus, anal canal, distal urethra and vagina, and urinary epithelium cells (lining urinary bladder and urinary ducts).

Exemplary cell types further include, but are not limited to, exocrine secretory epithelial cells such as salivary gland mucous cells (polysaccharide-rich secretion), salivary gland serous cells (glycoprotein enzyme-rich secretion), Von Ebner's gland cells in tongue (washes taste buds), mammary gland cells (milk secretion), lacrimal gland cells (tear secretion), Ceruminous gland cells in ear (wax secretion), Eccrine sweat gland dark cells (glycoprotein secretion), Eccrine sweat gland clear cells (small molecule secretion), Apocrine sweat gland cells (odoriferous secretion, sex-hormone sensitive), Gland of Moll cells in eyelid (specialized sweat gland), Sebaceous gland cells (lipid-rich sebum secretion) Bowman's gland cells in nose (washes olfactory epithelium), Brunner's gland cells in duodenum (enzymes and alkaline mucus), seminal vesicle cells (secretes seminal fluid components, including fructose for swimming sperm), prostate gland cells (secretes seminal fluid components), Bulbourethral gland cells (mucus secretion), Bartholin's gland cells (vaginal lubricant secretion), gland of Littre cells (mucus secretion), Uterus endometrium cells (carbohydrate secretion), isolated goblet cells of respiratory and digestive tracts (mucus secretion), stomach lining mucous cells (mucus secretion), gastric gland zymogenic cells (pepsinogen secretion), gastric gland oxyntic cells (hydrochloric acid secretion), pancreatic acinar cells (bicarbonate and digestive enzyme secretion), Paneth cells of small intestine (lysozyme secretion), type II pneumocytes of lung (surfactant secretion), and Clara cells of lung.

Exemplary cell types further include, but are not limited to, hormone secreting cells such as anterior pituitary cells (somatotropes, lactotropes, thyrotropes, gonadotropes, corticotropes), intermediate pituitary cells (secreting melanocyte-stimulating hormone), magnocellular neurosecretory cells (secreting oxytocin, secreting vasopressin), gut and respiratory tract cells secreting serotonin (secreting endorphin, secreting somatostatin, secreting gastrin, secreting secretin, secreting cholecystokinin, secreting insulin, secreting glucagons, secreting bombesin), thyroid gland cells (thyroid epithelial cells, parafollicular cells), parathyroid gland cells (parathyroid chief cells, oxyphil cells), adrenal gland cells (chromaffin cells, secreting steroid hormones), Leydig cells of testes secreting testosterone, Theca interna cells of ovarian follicle secreting estrogen, Corpus luteum cells of ruptured ovarian follicle secreting progesterone, kidney juxtaglomerular apparatus cells (renin secretion), macula densa cells of kidney, peripolar cells of kidney, and mesangial cells of kidney.

Exemplary cell types further include, but are not limited to, gut, exocrine glands and urogenital tract cells such as intestinal brush border cells (with microvilli), exocrine gland striated duct cells, gall bladder epithelial cells, kidney proximal tubule brush border cells, kidney distal tubule cells, ductulus efferens nonciliated cells, epididymal principal cells, and epididymal basal cells.

Exemplary cell types further include, but are not limited to, metabolism and storage cells such as hepatocytes (liver cells), white fat cells, brown fat cells, and liver lipocytes. Exemplary cell types further include, but are not limited to, barrier function cells (lung, gut, exocrine glands and urogenital tract) such as type I pneumocytes (lining air space of lung), pancreatic duct cells (centroacinar cell), nonstriated duct cells (of sweat gland, salivary gland, mammary gland, etc.), kidney glomerulus parietal cells, kidney glomerulus podocytes, loop of Henle thin segment cells (in kidney), kidney collecting duct cells, and duct cells (of seminal vesicle, prostate gland, etc.).

Exemplary cell types further include, but are not limited to, epithelial cells lining closed internal body cavities such as blood vessel and lymphatic vascular endothelial fenestrated cells, blood vessel and lymphatic vascular endothelial continuous cells, blood vessel and lymphatic vascular endothelial splenic cells, synovial cells (lining joint cavities, hyaluronic acid secretion), serosal cells (lining peritoneal, pleural, and pericardial cavities), squamous cells (lining perilymphatic space of ear), squamous cells (lining endolymphatic space of ear), columnar cells of endolymphatic sac with microvilli (lining endolymphatic space of ear), columnar cells of endolymphatic sac without microvilli (lining endolymphatic space of ear), dark cells (lining endolymphatic space of ear), vestibular membrane cells (lining endolymphatic space of ear), stria vascularis basal cells (lining endolymphatic space of ear), stria vascularis marginal cells (lining endolymphatic space of ear), cells of Claudius (lining endolymphatic space of ear), cells of Boettcher (lining endolymphatic space of ear), Choroid plexus cells (cerebrospinal fluid secretion), pia-arachnoid squamous cells, pigmented ciliary epithelium cells of eye, nonpigmented ciliary epithelium cells of eye, and corneal endothelial cells

Exemplary cell types further include, but are not limited to, ciliated cells with propulsive function such as respiratory tract ciliated cells, oviduct ciliated cells (in female), uterine endometrial ciliated cells (in female), rete testis cilated cells (in male), ductulus efferens ciliated cells (in male), and ciliated ependymal cells of central nervous system (lining brain cavities).

Exemplary cell types further include, but are not limited to, cxtracellular matrix secretion cells such as ameloblast epithelial cells (tooth enamel secretion), planum semilunatum epithelial cells of vestibular apparatus of ear (proteoglycan secretion), organ of Corti interdental epithelial cells (secreting tectorial membrane covering hair cells) loose connective tissue fibroblasts, corneal fibroblasts, tendon fibroblasts, bone marrow reticular tissue fibroblasts, pericytes, nucleus pulposus cells of intervertebral disc, cementoblast/cementocytes (tooth root bonelike cementum secretion), odontoblast/odontocyte (tooth dentin secretion), hyaline cartilage chondrocytes fibrocartilage chondrocytes, elastic cartilage chondrocytes, osteoblasts/osteocytes, osteoprogenitor cells (stem cell of osteoblasts), hyalocyte of vitreous body of eye, and stellate cells of perilymphatic space of ear.

Exemplary cell types further include, but are not limited to, contractile cells such as red skeletal muscle cells (slow), white skeletal muscle cells (fast), intermediate skeletal muscle cells, nuclear bag cells of Muscle spindle, nuclear chain cells of Muscle spindle, satellite cells (stem cell), ordinary heart muscle cells, nodal heart muscle cells, purkinje fiber cells, smooth muscle cells (various types), myoepithelial cells of iris, myoepithelial cells of exocrine glands, and red blood cells.

Exemplary cell types further include, but are not limited to, blood and immune system cells such as erythrocytes (red blood cell), megakaryocytes (platelet precursor), monocytes, connective tissue macrophages (various types), epidermal Langerhans cells, osteoclasts (in bone), dendritic cells (in lymphoid tissues), microglial cells (in central nervous system), neutrophil granulocytes, eosinophil granulocytes, basophil granulocytes, mast cells, helper T cells, suppressor T cells, cytotoxic T cells, B cells, natural killer cells, and reticulocytes.

Exemplary cell types further include, but are not limited to, sensory transducer cells such as auditory inner hair cells of organ of Corti, auditory outer hair cells of organ of Corti, basal cells of olfactory epithelium (stem cell for olfactory neurons), cold-sensitive primary sensory neurons, heat-sensitive primary sensory neurons, merkel cell of epidermis (touch sensor), olfactory receptor neurons, photoreceptor rod cell of eyes, photoreceptor blue-sensitive cone cells of eye, photoreceptor green-sensitive cone cells of eye, photoreceptor red-sensitive cone cells of eye, type I carotid body cells (blood pH sensor), Type II carotid body cells (blood pH sensor), type I hair cells of vestibular apparatus of ear (acceleration and gravity), type II hair cells of vestibular apparatus of ear (acceleration and gravity), and type I taste bud cells.

Exemplary cell types further include, but are not limited to, autonomic neuron cells such as cholinergic neural cells, adrenergic neural cells, and peptidergic neural cells. Exemplary cell types further include, but are not limited to, sense organ and peripheral neuron supporting cells such as inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, type I taste bud supporting cells, olfactory epithelium supporting cells, Schwann cells, satellite cells (encapsulating peripheral nerve cell bodies), and enteric glial cells.

Exemplary cell types further include, but are not limited to, central nervous system neurons and glial cells such as astrocytes, neuron cells, oligodendrocytes, and spindle neurons. Exemplary cell types further include, but are not limited to, lens cells such as anterior lens epithelial cells, crystallin-containing lens fiber cells, and karan cells. Exemplary cell types further include, but are not limited to, pigment cells such as melanocytes and retinal pigmented epithelial cells. Exemplary cell types further include, but are not limited to,

germ cells such as oogoniums/oocytes, spermatids, spermatocytes, spermatogonium cells, (stem cell for spermatocyte), and spermatozoon. Exemplary cell types further include, but are not limited to, nurse cells such as ovarian follicle cells, sertoli cells (in testis), and thymus epithelial cells. For more reference on cell types see Freitas Jr., 1999, Nanomedicine, Volume I: Basic Capabilities, Landes Bioscience, Georgetown, Tex.

### 5.3 Exemplary Disease States

In some embodiments, such as the method disclosed in FIGS. 2A and 2B, compound combinations are identified that affect a phenotypic of interest. In some embodiment the phenotype of interest is a disease state. As used herein, the term “disease state” refers to the presence or stage of disease in a biological specimen and/or a subject from which the biological specimen was obtained.

In some embodiments, the phenotype of interest is a lymphoid malignancy. Lymphoma is complex, thus application of a true systems biology perspective provided herein advantageously affords new opportunities to identify common signaling pathway defects that will allow for the development of a compound therapy with broad efficacy in the disease. While the relative market caps for these diseases appears small, it is clear that identifying drugs with niche applications, even in relatively rare sub-types of the disease, can offer a very promising strategy for getting agents approved at the FDA. This diversity works to the benefit of our commercialization potential.

In some embodiments, the phenotype of interest is breast cancer. Given the nature of the cytotoxic drugs available for the treatment of breast cancer, the enormous toll it places on families and patients, the toxicity of many of the conventional therapies and the incurability of metastatic disease, there is clearly a need to identify more disease specific and efficacious drugs for breast cancer. The development of targeted agents affecting the critical growth and survival pathways in breast cancer will afford new opportunities to improve the outcome of women with the disease, while simultaneously reducing the toxicity associated with many conventional treatment programs.

Additional exemplary disease states include, but are not limited to, asthma, ataxia telangiectasia (Jaspers and Bootsma, 1982, Proc. Natl. Acad. Sci. U.S.A. 79: 2641), bipolar disorder, a cancer, common late-onset Alzheimer's disease, diabetes, heart disease, hereditary early-onset Alzheimer's disease (George-Hyslop et al., 1990, Nature 347: 194), hereditary nonpolyposis colon cancer, hypertension, infection, maturity-onset diabetes of the young (Barbosa et al., 1976, Diabete Metab. 2: 160), mellitus, migraine, nonalcoholic fatty liver (NAFL) (Younossi, et al., 2002, Hepatology 35, 746-752), nonalcoholic steatohepatitis (NASH) (James & Day, 1998, J. Hepatol. 29: 495-501), non-insulin-dependent diabetes mellitus, obesity, polycystic kidney disease (Reeders et al., 1987, Human Genetics 76: 348), psoriases, schizophrenia, steatohepatitis and xeroderma pigmentosum (De Weerd-Kastelein, Nat. New Biol. 238: 80). Genetic heterogeneity hampers genetic mapping, because a chromosomal region may cosegregate with a disease in some families but not in others.

Auto-immune and immune disease states include, but are not limited to, Addison's disease, ankylosing spondylitis, antiphospholipid syndrome, Barth syndrome, Graves' Disease, hemolytic anemia, IgA nephropathy, lupus erythematosus, microscopic polyangiitis, multiple sclerosis, myasthenia gravis, myositis, osteoporosis, pemphigus, psoriasis, rheumatoid arthritis, sarcoidosis, scleroderma, and Sjogren's syndrome. Cardiology disease states include, but are not limited to, arrhythmia, cardiomyopathy, coronary artery disease, angina pectoris, and pericarditis.

Cancers addressed by the systems and the methods disclosed herein include, but are not limited to, sarcoma or carcinoma. Examples of such cancers include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, and heavy chain disease.

### 5.4 Exemplary Preprocessing Routines

Optionally, a number of different preprocessing routines can be performed to prepare MAPs for use in the methods disclosed above in conjunction with steps 204 and 206 of FIG. 2. Some such preprocessing protocols are described in this section. Typically, the preprocessing comprises normalizing the cellular constituent abundance measurement of each cellular constituent in a plurality of cellular constituents that is measured in a cell line. Many of the preprocessing protocols described in this section are used to normalize MAP data and are called normalization protocols. It will be appreciated that there are many other suitable normalization protocols that may be used in accordance with the system and method disclosed herein. Many of the normalization protocols found in this section are found in publicly available software, such as Microarray Explorer (Image Processing Section, Laboratory of Experimental and Computational Biology, National Cancer Institute, Frederick, Md. 21702, USA).

One normalization protocol is Z-score of intensity. In this protocol, cellular constituent abundance values are normalized by the (mean intensity)/(standard deviation) of raw intensities for all spots in a sample. For MAP data that is Gene Expression Profile (GEP) microarray data, the Z-score of intensity method normalizes each hybridized sample by the mean and standard deviation of the raw intensities for all of the spots in that sample. The mean intensity mnIᵢ and the standard deviation sdIᵢ are computed for the raw intensity of control genes. It is useful for standardizing the mean (to 0.0) and the range of data between hybridized samples to about −3.0 to +3.0. When using the Z-score, the Z differences (Z_{diff}) are computed rather than ratios. The Z-score intensity (Z-scoreᵢⱼ) for intensity Iᵢⱼ for probe i (hybridization probe, protein, or other binding entity) and spot j is computed as:

Z-scoreᵢⱼ=(Iᵢⱼ−mnIᵢ)/sdIᵢ,

and

Zdiffⱼ(x,y)=Z-scoreₓⱼ−Z-score_{yj}

where x represents the x channel and y represents the y channel.

Another normalization protocol is the median intensity normalization protocol in which the raw intensities for all spots in each sample are normalized by the median of the raw intensities. For GEP data, the median intensity normalization method normalizes each hybridized sample by the median of the raw intensities of control genes (medianIᵢ) for all of the spots in that sample. Thus, upon normalization by the median intensity normalization method, the raw intensity Iᵢⱼ for probe i and spot j, has the value Imᵢⱼ where,

Imᵢⱼ=(Iᵢⱼ/medianIᵢ).

Another normalization protocol is the log median intensity protocol. In this protocol, raw expression intensities are normalized by the log of the median scaled raw intensities of representative spots for all spots in the sample. For GEP data, the log median intensity method normalizes each hybridized sample by the log of median scaled raw intensities of control genes (medianIᵢ) for all of the spots in that sample. As used herein, control genes are a set of genes that have reproducible accurately measured expression values. The value 1.0 is added to the intensity value to avoid taking the log(0.0) when intensity has zero value. Upon normalization by the median intensity normalization method, the raw intensity Iᵢⱼ for probe i and spot j, has the value Imᵢⱼ where,

Imᵢⱼ=log(1.0+(Iᵢⱼ/medianIᵢ)).

Yet another normalization protocol is the Z-score standard deviation log of intensity protocol. In this protocol, raw expression intensities are normalized by the mean log intensity (mnLIᵢ) and standard deviation log intensity (sdLIᵢ). For GEP data, the mean log intensity and the standard deviation log intensity is computed for the log of raw intensity of control genes. Then, the Z-score intensity ZlogSᵢⱼ for probe i and spot j is:

Z log Sᵢⱼ=(log(Iᵢⱼ)−mnLIᵢ)/sdLIⱼ.

Still another normalization protocol is the Z-score mean absolute deviation of log intensity protocol. In this protocol, raw intensities are normalized by the Z-score of the log intensity using the equation (log(intensity)−mean logarithm)/standard deviation logarithm. For GEP data, the Z-score mean absolute deviation of log intensity protocol normalizes each bound sample by the mean and mean absolute deviation of the logs of the raw intensities for all of the spots in the sample. The mean log intensity mnLIᵢ and the mean absolute deviation log intensity madLIᵢ are computed for the log of raw intensity of control genes. Then, the Z-score intensity ZlogAᵢⱼ for probe i and spot j is:

Z log Aᵢⱼ=(log(Iᵢⱼ)−mnLIᵢ)/madLIᵢ.

Another normalization protocol is the user normalization gene set protocol. In this protocol, raw expression intensities are normalized by the sum of the genes in a user defined gene set in each sample. This method is useful if a subset of genes has been determined to have relatively constant expression across a set of samples. Yet another normalization protocol is the calibration DNA gene set protocol in which each sample is normalized by the sum of calibration DNA genes. As used herein, calibration DNA genes are genes that produce reproducible expression values that are accurately measured. Such genes tend to have the same expression values on each of several different GEPs. The algorithm is the same as user normalization gene set protocol described above, but the set is predefined as the genes flagged as calibration DNA.

Yet another normalization protocol is the ratio median intensity correction protocol. This protocol is useful in embodiments in which a two-color fluorescence labeling and detection scheme is used. In the case where the two fluors in a two-color fluorescence labeling and detection scheme are Cy3 and Cy5, measurements are normalized by multiplying the ratio (Cy3/Cy5) by medianCy5/medianCy3 intensities. If background correction is enabled, measurements are normalized by multiplying the ratio (Cy3/Cy5) by (medianCy5-medianBkgdCy5)/(medianCy3-medianBkgdCy3) where medianBkgd means median background levels.

In some embodiments, intensity background correction is used to normalize measurements. The background intensity data from a spot quantification programs may be used to correct spot intensity. Background may be specified as either a global value or on a per-spot basis. If the array images have low background, then intensity background correction may not be necessary.

An intensity dependent normalization can be implemented in R, a language and environment for statistical computing and graphics. In a specific embodiment, the normalization method uses a lowess( ) scatter plot smoother that can be applied to all or a subgroup of probes on the array. For a description of lowess( ), see, e.g., Becker et al., “The New S Language,” Wadsworth and Brooks/Cole (S version), 1988; Ripley, 1996, Pattern Recognition and Neural Networks, Cambridge University Press; and Cleveland, 1979, J. Amer. Statist. Assoc. 74, 829:836, each of which is hereby incorporated by reference in its entirety.

### 5.5 Transcriptional State Measurements

This section provides some exemplary methods for measuring the expression level of gene products, which are one type of cellular constituent that can be measures in steps 204 and 206 in order to obtain MAPs data. One of skill in the art will appreciate that measurement methods can be used in the systems and methods disclosed herein.

### 5.5.1 Transcript Assay Using Microarrays

The techniques described in this section are particularly useful for the determination of the expression state or the transcriptional state of a cell or cell type or any other biological sample. These techniques include the provision of polynucleotide probe arrays that can be used to provide simultaneous determination of the expression levels of a plurality of genes. These techniques further provide methods for designing and making such polynucleotide probe arrays.

The expression level of a nucleotide sequence of a gene can be measured by any high throughput technique. However measured, the result is either the absolute or relative amounts of transcripts or response data including, but not limited to, values representing abundances or abundance ratios. Preferably, measurement of the microarray profile is made by hybridization to transcript arrays, which are described in this subsection. In one embodiment microarrays such as “transcript arrays” or “profiling arrays” are used. Transcript arrays can be employed for analyzing the microarray profile in a cell sample and especially for measuring the microarray profile of a cell sample of a particular tissue type or developmental state or exposed to a drug of interest.

In one embodiment, a molecular profile is an microarray profile that is obtained by hybridizing detectably labeled polynucleotides representing the nucleotide sequences in mRNA transcripts present in a cell (e.g., fluorescently labeled cDNA synthesized from total cell mRNA) to a microarray. In some embodiments, a microarray is an array of positionally-addressable binding (e.g., hybridization) sites on a support for representing many of the nucleotide sequences in the genome of a cell or organism, preferably most or almost all of the genes. Each of such binding sites consists of polynucleotide probes bound to the predetermined region on the support. Microarrays can be made in a number of ways, of which several are described herein below. However produced, microarrays share certain characteristics. The arrays are reproducible, allowing multiple copies of a given array to be produced and easily compared with each other.

Preferably, a given binding site or unique set of binding sites in the microarray will specifically bind (e.g., hybridize) to a nucleotide sequence in a single gene from a cell or organism (e.g., to exon of a specific mRNA or a specific cDNA derived therefrom). The microarrays used can include one or more test probes, each of which has a polynucleotide sequence that is complementary to a subsequence of RNA or DNA to be detected. Each probe typically has a different nucleic acid sequence, and the position of each probe on the solid surface of the array is usually known. Indeed, the microarrays are preferably addressable arrays, more preferably positionally addressable arrays. Each probe of the array is preferably located at a known, predetermined position on the solid support so that the identity (e.g., the sequence) of each probe can be determined from its position on the array (e.g., on the support or surface). In some embodiments, the arrays are ordered arrays.

Preferably, the density of probes on a microarray or a set of microarrays is 100 different (e.g., non-identical) probes per 1 cm² or higher. In some embodiments, a microarray can have at least 550 probes per 1 cm², at least 1,000 probes per 1 cm², at least 1,500 probes per 1 cm² or at least 2,000 probes per 1 cm². In some embodiments, the microarray is a high density array, preferably having a density of at least 2,500 different probes per 1 cm². A microarray can contain at least 2,500, at least 5,000, at least 10,000, at least 15,000, at least 20,000, at least 25,000, at least 50,000 or at least 55,000 different (e.g., non-identical) probes.

In one embodiment, the microarray is an array (e.g., a matrix) in which each position represents a discrete binding site for a nucleotide sequence of a transcript encoded by a gene (e.g., for an exon of an mRNA or a cDNA derived therefrom). In such and embodiment, the collection of binding sites on a microarray contains sets of binding sites for a plurality of genes. For example, in various embodiments, a microarray can comprise binding sites for products encoded by fewer than 50% of the genes in the genome of an organism. Alternatively, a microarray can have binding sites for the products encoded by at least 50%, at least 75%, at least 85%, at least 90%, at least 95%, at least 99% or 100% of the genes in the genome of an organism (e.g., human, mammal, rat, mouse, pig, dog, cat, etc.). In other embodiments, a microarray can having binding sites for products encoded by fewer than 50%, by at least 50%, by at least 75%, by at least 85%, by at least 90%, by at least 95%, by at least 99% or by 100% of the genes expressed by a cell of an organism. The binding site can be a DNA or DNA analog to which a particular RNA can specifically hybridize. The DNA or DNA analog can be, e.g., a synthetic oligomer or a gene fragment, e.g. corresponding to an exon.

In some embodiments, a gene or an exon in a gene is represented in the profiling arrays by a set of binding sites comprising probes with different polynucleotides that are complementary to different sequence segments of the gene or the exon. Such polynucleotides are preferably of the length of 15 to 200 bases, more preferably of the length of 20 to 100 bases, most preferably 40-60 bases. In some embodiments, the profiling arrays comprise one probe specific to each target gene or exon. However, if desired, the profiling arrays can contain at least 2, 5, 10, 100, or 1000 or more probes specific to some target genes or exons.

### 5.5.1.1 Preparing Probes for Microarrays

As noted above, the “probe” to which a particular polynucleotide molecule, such as an exon, specifically hybridizes is a complementary polynucleotide sequence. Preferably one or more probes are selected for each target exon. For example, when a minimum number of probes are to be used for the detection of an exon, the probes normally comprise nucleotide sequences greater than 40 bases in length. Alternatively, when a large set of redundant probes is to be used for an exon, the probes normally comprise nucleotide sequences of 40-60 bases. The probes can also comprise sequences complementary to full length exons. The lengths of exons can range from less than 50 bases to more than 200 bases. Therefore, when a probe length longer than exon is to be used, it is preferable to augment the exon sequence with adjacent constitutively spliced exon sequences such that the probe sequence is complementary to the continuous mRNA fragment that contains the target exon. This will allow comparable hybridization stringency among the probes of an exon profiling array. It will be understood that each probe sequence may also comprise linker sequences in addition to the sequence that is complementary to its target sequence.

In some embodiments, the probes may comprise DNA or DNA “mimics” (e.g., derivatives and analogues) corresponding to a portion of each exon of each gene in an organism's genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of exon segments from genomic DNA, cDNA (e.g., by RT-PCR), or cloned sequences. PCR primers are preferably chosen based on known sequence of the exons or cDNA that result in amplification of unique fragments (e.g., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between 10 and 600 bases in length, more typically between 20 and 100 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; and U.S. Pat. No. 5,539,083).

In alternative embodiments, the hybridization sites (e.g., the probes) are made from plasmid or phage clones of genes, cDNAs (e.g., expressed sequence tags), or inserts therefrom (Nguyen et al., 1995, Genomics 29:207-209).

### 5.5.1.2 Attaching Nucleic Acids to the Solid Surface

Preformed polynucleotide probes can be deposited on a support to form the array. Alternatively, polynucleotide probes can be synthesized directly on the support to form the array. The probes are attached to a solid support or surface, which may be made, e.g., from glass, plastic (e.g., polypropylene, nylon), polyacrylamide, nitrocellulose, gel, or other porous or nonporous material.

One method for attaching the nucleic acids to a surface is by printing on glass plates, as is described generally by Schena et al, 1995, Science 270:467-470. This method is especially useful for preparing microarrays of cDNA (See also, DeRisi et al, 1996, Nature Genetics 14:457-460; Shalon et al., 1996, Genome Res. 6:639-645; and Schena et al., 1995, Proc. Natl. Acad. Sci. U.S.A. 93:10539-11286).

A second method for making microarrays is by making high-density polynucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. The array produced can be redundant, with several polynucleotide molecules per exon.

Other methods for making microarrays, e.g., by masking (Maskos and Southern, 1992, Nucl. Acids. Res. 20:1679-1684), may also be used. In principle, and as noted supra, any type of array, for example, dot blots on a nylon hybridization membrane (see Sambrook et al., supra) could be used.

In one embodiment, microarrays are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123; and U.S. Pat. No. 6,028,189 to Blanchard. Specifically, the polynucleotide probes in such microarrays can be synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Polynucleotide probes are normally attached to the surface covalently at the 3N end of the polynucleotide. Alternatively, polynucleotide probes can be attached to the surface covalently at the 5N end of the polynucleotide (see for example, Blanchard, 1998, in Synthetic DNA Arrays in Genetic Engineering, Vol. 20, J. K. Setlow, Ed., Plenum Press, New York at pages 111-123).

### 5.5.1.3 Target Polynucleotide Molecules

Target polynucleotides that can be analyzed include RNA molecules such as, but by no means limited to, messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides that can also be analyzed include, but are not limited to DNA molecules such as genomic DNA molecules, cDNA molecules, and fragments thereof including oligonucleotides, ESTs, STSs, etc.

The target polynucleotides can be from any source. For example, the target polynucleotide molecules can be naturally occurring nucleic acid molecules such as genomic or extragenomic DNA molecules isolated from a patient, or RNA molecules, such as mRNA molecules, isolated from a patient. Alternatively, the polynucleotide molecules can be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In some embodiments, the target polynucleotides will correspond to particular genes or to particular gene transcripts (e.g., to particular mRNA sequences expressed in cells or to particular cDNA sequences derived from such mRNA sequences). However, in many embodiments, the target polynucleotides can correspond to particular fragments of a gene transcript. For example, the target polynucleotides may correspond to different exons of the same gene, e.g., so that different splice variants of the gene can be detected and/or analyzed.

In some embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest using guanidinium thiocyanate lysis followed by CsCl centrifugation and an oligo dT purification (Chirgwin et al., 1979, Biochemistry 18:5294-5299). In another embodiment, RNA is extracted from cells using guanidinium thiocyanate lysis followed by purification on RNeasy columns (Qiagen). cDNA is then synthesized from the purified mRNA using, e.g., oligo-dT or random primers. In some embodiments, the target polynucleotides are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. Pat. Nos. 6,271,002, and 7,229,765. Both oligo-dT primers (U.S. Pat. Nos. 5,545,522 and 6,132,997) or random primers (U.S. Pat. No. 7,229,765) that contain an RNA polymerase promoter or complement thereof can be used. The target polynucleotides can be short and/or fragmented polynucleotide molecules that are representative of the original nucleic acid population of the cell.

The target polynucleotides to be analyzed are typically detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

In some instances, the detectable label is a fluorescent label, e.g., by incorporation of nucleotide analogs. Other labels suitable for use include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Some radioactive isotopes include, but are not limited to, ³²P, ³⁵S, ¹⁴C, ¹⁵N and ¹²⁵I. Fluorescent molecules include, but are not limited to, fluorescein and its derivatives, rhodamine and its derivatives, texas red, 5N carboxy-fluorescein (“FMA”), 2N,7N-dimethoxy-4N,5N-dichloro-6-carboxy-fluorescein (“JOE”), N,N,NN,NN-tetramethyl-6-carboxy-rhodamine (“TAMRA”), 6Ncarboxy-X-rhodamine (“ROX”), HEX, TET, IRD40, and IRD41. Fluorescent molecules further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable, but are not limited to, ferritin, hemocyanin, and colloidal gold. Alternatively, in some embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

### 5.5.1.4 Hybridization to Microarrays

As described supra, nucleic acid hybridization and wash conditions are chosen so that the polynucleotide molecules to be analyzed (referred to herein as the “target polynucleotide molecules) specifically bind or specifically hybridize to the complementary polynucleotide sequences of the array, preferably to a specific array site, where its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (e.g., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization with Nucleic Acid Probes, Elsevier Science Publishers B.V. and Kricka, 1992, Nonisotopic DNA Probe Techniques, Academic Press, San Diego, Calif.

Exemplary hybridization conditions for use with the screening and/or signaling chips include hybridization at a temperature at or near the mean melting temperature of the probes (e.g., within 5° C., more preferably within 2° C.) in 1 M NaCl, 50 mM MES buffer (pH 6.5), 0.5% sodium Sarcosine and 30% formamide.

### 5.5.1.5 Signal Detection and Data Analysis

It will be appreciated that when target sequences, e.g., cDNA or cRNA, complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (e.g., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal.

When fluorescently labeled probes are used, the fluorescence emissions at each site of a transcript array can be, preferably, detected by scanning confocal laser microscopy. In one embodiment, a separate scan, using the appropriate excitation line, is carried out for each of two fluorophores used in such embodiments. Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In some embodiments, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, can be used to monitor mRNA abundance levels at a large number of sites simultaneously.

Signals are recorded and, in a preferred embodiment, analyzed by computer. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors can be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

### 5.6 Apparatus, Computer and Computer Program Product Implementations

The present invention can be implemented as a computer program product that comprises a computer program mechanism embedded in a computer-readable storage medium. Further, any of the methods disclosed herein can be implemented in one or more computers or other forms of apparatus. Examples of apparatus include but are not limited to, a computer, and a spectroscopic measuring device (e.g., a microarray reader or microarray scanner). Further still, any of the methods disclosed herein can be implemented in one or more computer program products. Some embodiments disclosed herein provide a computer program product that encodes any or all of the methods disclosed herein. Such methods can be stored on a CD-ROM, DVD, magnetic disk storage product, or any other computer-readable data or program storage product. Such methods can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices.

Some embodiments provide a computer program product that contains any or all of the program modules shown in FIG. 1. These program modules can be stored on a CD-ROM, DVD, magnetic disk storage product, or any other computer-readable data or program storage product. The program modules can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices.

### 5.7 Exemplary Cell Based Assays

The cell-based assays that can be used can range from cytotoxic assays including apoptosis to cell proliferation and metabolic assays. Cell-based assays can also include high throughput screening assays and other custom bioassays used to characterize drug stability, drug potency and drug selectivity. In some embodiments, cell-based assays encompass testing whole cells in a variety of formats including ELISA and immunohistochemical methods. In some embodiments cell-based assays are prepared by growing and differentiating stem cells to monitor stem cell differentiation in the present of specific compounds.

In some embodiments high throughput cell-based assays are screened for response to each compound in one or more libraries of compounds. In some instances in accordance with such embodiments, a frozen stock of a predetermined cell line is generated at the onset of any high throughput screening assay to maintain reproducibility of the desired bioactivity. In some embodiments the initial design of the assay is performed with a 96, 384 or 1536 well plate with a read out that is fluorescence, luminescence, calorimetric or radioactivity depending upon the variable to be measured. This enables microscopic visualization of the cells. In some embodiments, morphologic information on the status of the culture and individual cells is used.

In some embodiments, cell growth is measured in cell-based assays. For example, in some embodiments cell growth is measured by a homogeneous, vital dye method in which one of several choices of dye is added to cells in a 96, 384 or 1536 well plate (or other form of plate), incubated for increasing hours, and read directly in a plate reader. The dye is enzymatically changed in healthy cells so that development of color or fluorescence is measured using a different wavelength than the unaltered dye. Addition of a growth factor, an inhibitor or a cytotoxic factor to cells is easily read. Alternatively, uptake of 3^{H}-thymidine is used specifically for assay of DNA synthesis, or as a more sensitive assay of cell proliferation for slow growing cells

Cell death occurs by lysis, necrosis, or apoptosis. Lysis is the destruction of the cell surface membrane such as by the action of an antibody and complement that makes holes in the membrane. Necrosis occurs through the action of toxic factors that act within the cell, such as irreversible inhibitors of protein, RNA or DNA synthesis, or mitotic poisons. Apoptosis is a programmed cell death used by the body to remove damaged or unwanted cells, and occurs during cytotoxic T cell killing and with some cancer chemotherapies. Apoptosis is characterized by early events such as expression of phosphotidylserine on the cell surface and fragmentation of the DNA, followed by loss of membrane integrity and mitochondrial function. In some embodiments, cell death is assessed microscopically by uptake of trypan blue dye that is excluded by live cells. The percentage of dying cells is determined microscopically or by flow cytometry using vital stains or DNA-binding dyes. In some embodiments, high throughput measurement of cell death is performed by release of a label from cells prelabeled with a radiotracer, typically 51 Cr, or a fluorescent or color marker. Alternatively, fluorescent or calorimetric dye methods are used.

In some embodiments, a cell-based assay is used to study drug effect on metabolism. This can be measured by radioactive precursor uptake, thymidine, uridine (or uracil for bacteria), and amino acid, into DNA, RNA and proteins. Carbohydrate or lipid synthesis is similarly measured using suitable precursors. Turnover of nucleic acid or protein or the degradation of specific cell components, is measured by prelabeling (or pulse labeling) followed by a purification step and quantitation of remaining label or sometimes by measurement of chemical amounts of the component. Energy source metabolism is also analyzed for optimal cell growth.

In some embodiments, flow cytometry is used to conduct cell-based assays. Flow cytometry allows the study of individual live cells in a population of 10⁴-10⁵ cells, with the detection stage requiring less than a minute. Specific cell components are stained by fluorescent antibodies or other reagents. Cells can be made more permeable to large proteins without changing overall cell shape. Simultaneously, cell viability, cell size, and internal structures (e.g. distinguishing lymphocytes from granulocytes with many vesicles) can be measured. After cells are stained, and fixed with glutaraldehyde if desired, the cell suspension is distributed into droplets containing one cell or no cell. The droplets flow through a chamber with one or multiple laser beams for excitation of the fluorescent probes. The data are displayed as a histogram of cell numbers with increasing fluorescence signal, and can be transformed to show double (and triple, etc.) labeled cells and integration for the fraction of cells in any chosen window of signals. Additionally, a mixture of cells can be analyzed by cell size.

In some embodiments, phase and fluorescence microscopy is used to conduct cell-based assays. Light microscopy shows the general state of cells, and combined with trypan blue exclusion, the percent of viable cells. Small, optically dense cells indicate necrosis, while bloated “blasting” cells with blebs indicate apoptosis. Phase microscopy views cells in indirect light; the reflected light shows more detail, particularly intracellular structures. Fluorescence microscopy detects individual components in cells, after labeling with selective dyes or specific antibodies, and can distinguish cell surface from intracellular labeling. Microscopic observation of cell cultures is an integral tool for tissue culture, as it reveals the culture health during the maintenance, expansion and experimentation phases of the study.

A wide variety of protocols can be used to measure cytotoxicity in cell-based assays. In some embodiments, assay plates are set up containing cells and allowed to equilibrate for a predetermined period before adding test compounds. Alternatively, cells may be added directly to plates that already contain library compounds. The duration of exposure to the test compound may vary from less than an hour to several days, depending on specific project goals.

Brief periods (e.g., 10 hours or less, five hours or less, one hour or less, etc.) of exposure is used in some embodiments to determine if test compounds cause an immediate necrotic insult to cells, whereas exposure for several days is used in some embodiments to determine if test compounds cause an inhibition of cell proliferation. In some embodiments, cell viability or cytotoxicity measurements usually are determined at the end of the exposure period. Assays that require only a few minutes to generate a measurable signal (e.g., ATP quantitation or LDH-release assays) provide information representing a snapshot in time and have an advantage over assays that may require several hours of incubation to develop a signal (e.g., MTS or resazurin). In vitro cultured cells exist as a heterogeneous population. When populations of cells are exposed to test compounds they do not all respond simultaneously. Cells exposed to toxin may respond over the course of several hours or days, depending on many factors including the mechanism of cell death, the concentration of the toxin, and the duration of exposure. As a result of culture heterogeneity, the data from Some plate-based assay formats used in the methods disclosed herein represent an average of the signal from the population of cells.

An example of a cell-based assay system is the CELLTITER 96® Aqueous assay (Promega) that is based on the reduction of the tetrazolium salt, MTS, to a colored formazan compound by viable cells in culture. The MTS tetrazolium is similar to the widely used MTT tetrazolium. The formazan product of MTS reduction is soluble in cell culture medium. Metabolism in viable cells produces “reducing equivalents” such as NADH or NADPH. These reducing compounds pass their electrons to an intermediate electron transfer reagent that can reduce MTS into the aqueous, formazan product. Upon cell death, cells rapidly lose the ability to reduce tetrazolium products. The production of the colored formazan product, therefore, is proportional to the number of viable cells in culture.

Another example of a cell-based assay system is the CELLTITER 96® AQueous One Solution Cell Proliferation Assay which is an MTS-based assay that involves adding a reagent directly to the assay wells at a recommended ratio of 20 μl reagent to 100 μl of culture medium. Cells are incubated 1-4 hours at 37° C., and then absorbance is measured at 490 nm.

### 5.8 Exemplary Transcription Factors

Table 1 provides a nonlimiting list of exemplary human transcription factors may be used in the methods and systems disclosed herein. In some embodiments, any combination of transcription factors listed in Table 1 is used in the methods and systems disclosed herein. In some embodiments, any combination of transcription factors listed in Table 1 as well as transcription factors not listed in Table 1 is used in the methods and systems disclosed herein. In some embodiments, transcription factors not listed in Table 1 are used in the methods and systems disclosed herein. In Table 1, the field “GeneID” is the National Center for Biotechnology Information (NCBI) Entrez gene identifier for the gene.

Furthermore, the present invention is not limited to application to humans but may be used in other mammals, plants, yeast, or any other biological organisms. In such instances, transcription factors for such organisms would be used in preferred embodiments.

TABLE 1
Transcription Factors

| Transcription Factor Symbol(Name) | Gene ID |
|---|---|
| AATF (apoptosis antagonizing transcription factor) | 26574 |
| ABRA (actin-binding Rho activating protein) | 137735 |
| ABT1 (activator of basal transcription 1) | 29777 |
| ADNP (activity-dependent neuroprotector homeobox) | 23394 |
| ADNP2 (ADNP homeobox 2) | 22850 |
| AFF1 (AF4/FMR2 family, member 1) | 4299 |
| AFF4 (AF4/FMR2 family, member 4) | 27125 |
| AGT (angiotensinogen (serpin peptidase inhibitor, clade A, member 8)) | 183 |
| AHR (aryl hydrocarbon receptor) | 196 |
| AIRE (autoimmune regulator) | 326 |
| ALS2CR8 (amyotrophic lateral sclerosis 2 (juvenile) chromosome region)) | 79800 |
| ALX1 (ALX homeobox 1) | 8092 |
| ALX3 (ALX homeobox 3) | 257 |
| ALX4 (ALX homeobox 4) | 60529 |
| ANKRD30A (ankyrin repeat domain 30A) | 91074 |
| AR (androgen receptor) | 367 |
| ARGFX (arginine-fifty homeobox) | 503582 |
| ARID3A (AT rich interactive domain 3A (BRIGHT-like)) | 1820 |
| ARID4A (AT rich interactive domain 4A (RBP1-like)) | 5926 |
| ARNT (aryl hydrocarbon receptor nuclear translocator)) | 405 |
| ARNT2 (aryl-hydrocarbon receptor nuclear translocator 2) | 9915 |
| ARNTL (aryl hydrocarbon receptor nuclear translocator-like) | 406 |
| ARNTL2 (aryl hydrocarbon receptor nuclear translocator-like 2) | 56938 |
| ARX (aristaless related homeobox) | 170302 |
| ASCL1 (achaete-scute complex homolog 1 (Drosophila)) | 429 |
| ASCL2 (achaete-scute complex homolog 2 (Drosophila)) | 430 |
| ASH1L (ash1 (absent, small, or homeotic)-like (Drosophila)) | 55870 |
| ATAD2 (ATPase family, AAA domain containing 2) | 29028 |
| ATF1 (activating transcription factor 1) | 466 |
| ATF2 (activating transcription factor 2) | 1386 |
| ATF3 (activating transcription factor 3) | 467 |
| ATF4 (activating transcription factor 4 (tax-responsive enhancer element B67)) | 468 |
| ATF5 (activating transcription factor 5) | 22809 |
| ATF6 (activating transcription factor 6) | 22926 |
| ATF6B (activating transcription factor 6 beta) | 1388 |
| ATF7 (activating transcription factor 7) | 11016 |
| ATOH1 (atonal homolog 1 (Drosophila)) | 474 |
| BACH1 (BTB and CNC homology 1, basic leucine zipper transcription factor 1) | 571 |
| BACH2 (BTB and CNC homology 1, basic leucine zipper transcription factor 2) | 60468 |
| BARHL1 (BarH-like homeobox 1) | 56751 |
| BARHL2 (BarH-like homeobox 2) | 343472 |
| BARX1 (BARX homeobox 1) | 56033 |
| BARX2 (BARX homeobox 2) | 8538 |
| BATF (basic leucine zipper transcription factor, ATF-like) | 10538 |
| BATF2 (basic leucine zipper transcription factor, ATF-like 2) | 116071 |
| BATF3 (basic leucine zipper transcription factor, ATF-like 3) | 55509 |
| BAZ1B (bromodomain adjacent to zinc finger domain, 1B) | 9031 |
| BCL10 (B-cell CLL/lymphoma 10) | 8915 |
| BCL3 (B-cell CLL/lymphoma 3) | 602 |
| BCL6 (B-cell CLL/lymphoma 6) | 604 |
| BHLHE40 (basic helix-loop-helix family, member e40) | 8553 |
| BHLHE41 (basic helix-loop-helix family, member e41) | 79365 |
| BLZF1 (basic leucine zipper nuclear factor 1) | 8548 |
| BNC1 (basonuclin 1) | 646 |
| BRD8 (bromodomain containing 8) | 10902 |
| BRF1 (BRF1 homolog, subunit of RNA polymerase III transcription initiation factor | 2972 |
| TF3B90, TFIIIB90, hBRF) | |
| BSX (brain-specific homeobox) | 390259 |
| BTAF1 (BTAF1 RNA polymerase II, B-TFIID transcription factor-associated) | 9044 |
| BTF3 (basic transcription factor 3) | 689 |
| BTF3L2 (basic transcription factor 3, like 2) | 652963 |
| BTF3L3 (basic transcription factor 3, like 3) | 132556 |
| BUD31 (BUD31 homolog (S. cerevisiae)) | 8896 |
| C11orf9 (chromosome 11 open reading frame 9) | 745 |
| C14orf39 (chromosome 14 open reading frame 39) | 317761 |
| C21orf66 (chromosome 21 open reading frame 66) | 94104 |
| C2orf3 (chromosome 2 open reading frame 3) | 6936 |
| CAMK2A (calcium/calmodulin-dependent protein kinase II alpha) | 815 |
| CARD11 (caspase recruitment domain family, member 11) | 84433 |
| CAT (catalase) | 847 |
| CBFA2T2 (core-binding factor, runt domain, alpha subunit 2; translocated to, 2) | 9139 |
| CBFA2T3 (core-binding factor, runt domain, alpha subunit 2; translocated to, 3) | 863 |
| CBFB (core-binding factor, beta subunit) | 865 |
| CBL (Cas-Br-M (murine) ecotropic retroviral transforming sequence) | 867 |
| CCRN4L (CCR4 carbon catabolite repression 4-like (S. cerevisiae)) | 25819 |
| CDKN2A (cyclin-dependent kinase inhibitor 2A (melanoma, p16, inhibits CDK4)) | 1029 |
| CDX1 (caudal type homeobox 1) | 1044 |
| CDX2 (caudal type homeobox 2) | 1045 |
| CDX4 (caudal type homeobox 4) | 1046 |
| CEBPA (CCAAT/enhancer binding protein (C/EBP), alpha) | 1050 |
| CEBPB (CCAAT/enhancer binding protein (C/EBP), beta) | 1051 |
| CEBPD (CCAAT/enhancer binding protein (C/EBP), delta) | 1052 |
| CEBPE (CCAAT/enhancer binding protein (C/EBP), epsilon) | 1053 |
| CEBPG (CCAAT/enhancer binding protein (C/EBP), gamma) | 1054 |
| CIITA (class II, major histocompatibility complex, transactivator) | 4261 |
| CBF1 interacting corepressor) | 9541 |
| CITED1 (Cbp/p300-interacting transactivator, with Glu/Asp-rich carboxy-terminal | 4435 |
| domain, 1) | |
| CITED2 (Cbp/p300-interacting transactivator, with Glu/Asp-rich carboxy-terminal | 10370 |
| domain, 2) | |
| CLOCK (clock homolog (mouse)) | 9575 |
| CNBP (CCHC-type zinc finger, nucleic acid binding protein) | 7555 |
| CNOT7 (CCR4-NOT transcription complex, subunit 7) | 29883 |
| CNOT8 (CCR4-NOT transcription complex, subunit 8) | 9337 |
| COMMD7 (COMM domain containing 7) | 149951 |
| CREB1 (cAMP responsive element binding protein 1) | 1385 |
| CREB3 (cAMP responsive element binding protein 3 LZIP-alpha) | 10488 |
| CREB3L1 (cAMP responsive element binding protein 3-like 1) | 90993 |
| CREB3L2 (cAMP responsive element binding protein 3-like 2) | 64764 |
| CREB3L3 (cAMP responsive element binding protein 3-like 3) | 84699 |
| CREB3L4 (cAMP responsive element binding protein 3-like 4) | 148327 |
| CREB5 (cAMP responsive element binding protein 5) | 9586 |
| CREBBP (CREB binding protein) | 1387 |
| CREBL2 (cAMP responsive element binding protein-like 2) | 1389 |
| CREBZF (CREB/ATF bZIP transcription factor) | 58487 |
| CREG1 (cellular repressor of E1A-stimulated genes 1) | 8804 |
| CREM (cAMP responsive element modulator) | 1390 |
| CRKRS (Cdc2-related kinase, arginine/serine-rich) | 51755 |
| CRX (cone-rod homeobox) | 1406 |
| CSDA (cold shock domain protein A) | 8531 |
| CSRNP1 (cysteine-serine-rich nuclear protein 1) | 64651 |
| CSRNP2 (cysteine-serine-rich nuclear protein 2) | 81566 |
| CSRNP3 (cysteine-serine-rich nuclear protein 3) | 80034 |
| CTCF (CCCTC-binding factor (zinc finger protein)) | 10664 |
| CTNNB1 (catenin (cadherin-associated protein), beta 1, 88 kDa) | 1499 |
| CUX1 (cut-like homeobox 1) | 1523 |
| CUX2 (cut-like homeobox 2) | 23316 |
| DACH1 (dachshund homolog 1 (Drosophila)) | 1602 |
| DBP (D site of albumin promoter (albumin D-box) binding protein) | 1628 |
| DBX1 (developing brain homeobox 1) | 120237 |
| DBX2 (developing brain homeobox 2) | 440097 |
| DDIT3 (DNA-damage-inducible transcript 3) | 1649 |
| DEK (DEK oncogene) | 7913 |
| DLX1 (distal-less homeobox 1) | 1745 |
| DLX2 (distal-less homeobox 2) | 1746 |
| DLX3 (distal-less homeobox 3) | 1747 |
| DLX4 (distal-less homeobox 4) | 1748 |
| DLX5 (distal-less homeobox 5) | 1749 |
| DLX6 (distal-less homeobox 6) | 1750 |
| DMBX1 (diencephalon/mesencephalon homeobox 1) | 127343 |
| DMRT1 (doublesex and mab-3 related transcription factor 1) | 1761 |
| DMRT2 (doublesex and mab-3 related transcription factor 2) | 10655 |
| DMRT3 (doublesex and mab-3 related transcription factor 3) | 58524 |
| DMRTA1 (DMRT-like family A1) | 63951 |
| DMRTA2 (DMRT-like family A2) | 63950 |
| DMRTB1 (DMRT-like family B with proline-rich C-terminal, 1) | 63948 |
| DMRTC2 (DMRT-like family C2) | 63946 |
| DMTF1 (cyclin D binding myb-like transcription factor 1) | 9988 |
| DPRX (divergent-paired related homeobox) | 503834 |
| DRAP1 (DR1-associated protein 1 (negative cofactor 2 alpha)) | 10589 |
| DRGX (dorsal root ganglia homeobox factor DRG11) | 644168 |
| DUX1 (double homeobox, 1) | 26584 |
| DUX2 (double homeobox, 2) | 26583 |
| DUX3 (double homeobox, 3) | 26582 |
| DUX4 (double homeobox, 4) | 22947 |
| DUX5 (double homeobox, 5) | 26581 |
| DUXA (double homeobox A) | 503835 |
| E2F1 (E2F transcription factor 1) | 1869 |
| E2F2 (E2F transcription factor 2) | 1870 |
| E2F3 (E2F transcription factor 3) | 1871 |
| E2F4 (E2F transcription factor 4, p107/p130-binding) | 1874 |
| E2F5 (E2F transcription factor 5, p130-binding) | 1875 |
| E2F6 (E2F transcription factor 6) | 1876 |
| E2F7 (E2F transcription factor 7) | 144455 |
| E2F8 (E2F transcription factor 8) | 79733 |
| E4F1 (E4F transcription factor 1) | 1877 |
| EDA (ectodysplasin A ectodermal dysplasia protein) | 1896 |
| EDA2R (ectodysplasin A2 receptor) | 60401 |
| EDF1 (endothelial differentiation-related factor 1) | 8721 |
| EGLN1 (egl nine homolog 1 (C. elegans) | 54583 |
| EGR1 (early growth response 1) | 1958 |
| EGR2 (early growth response 2 (Krox-20 homolog, Drosophila)) | 1959 |
| EGR3 (early growth response 3) | 1960 |
| EGR4 (early growth response 4) | 1961 |
| EHF (ets homologous factor) | 26298 |
| ELF1 (E74-like factor 1 (ets domain transcription factor)) | 1997 |
| ELF2 (E74-like factor 2 (ets domain transcription factor related factor)) | 1998 |
| ELF3 (E74-like factor 3 (ets domain transcription factor, epithelial-specific)) | 1999 |
| ELF4 (E74-like factor 4 (ets domain transcription factor)) | 2000 |
| ELF5 (E74-like factor 5 (ets domain transcription factor)) | 2001 |
| ELK1 (ELK1, member of ETS oncogene family) | 2002 |
| ELK3 (ELK3, ETS-domain protein (SRF accessory protein 2)) | 2004 |
| ELK4 (ELK4, ETS-domain protein (SRF accessory protein 1)) | 2005 |
| ELL2 (elongation factor, RNA polymerase II, 2) | 22936 |
| EMX1 (empty spiracles homeobox 1) | 2016 |
| EMX2 (empty spiracles homeobox 2) | 2018 |
| EN1 (engrailed homeobox 1) | 2019 |
| EN2 (engrailed homeobox 2) | 2020 |
| ENO1 (enolase 1, (alpha)) | 2023 |
| EOMES (eomesodermin homolog (Xenopus laevis)) | 8320 |
| EP300 (E1A binding protein p300) | 2033 |
| EPAS1 (endothelial PAS domain protein 1) | 2034 |
| ERC1 (ELKS/RAB6-interacting/CAST family member 1) | 23085 |
| ERF (Ets2 repressor factor) | 2077 |
| ERG (v-ets erythroblastosis virus E26 oncogene homolog (avian)) | 2078 |
| ESR1 (estrogen receptor 1) | 2099 |
| ESR2 (estrogen receptor 2 (ER beta)) | 2100 |
| ESRRA (estrogen-related receptor alpha) | 2101 |
| ESRRB (estrogen-related receptor beta) | 2103 |
| ESRRG (estrogen-related receptor gamma) | 2104 |
| ESX1 (ESX homeobox 1) | 80712 |
| ETS1 (v-ets erythroblastosis virus E26 oncogene homolog 1 (avian)) | 2113 |
| ETS2 (v-ets erythroblastosis virus E26 oncogene homolog 2 (avian)) | 2114 |
| ETV1 (ets variant 1) | 2115 |
| ETV2 (ets variant 2) | 2116 |
| ETV3 (ets variant 3) | 2117 |
| ETV3L (ets variant 3-like) | 440695 |
| ETV4 (ets variant 4) | 2118 |
| ETV5 (ets variant 5) | 2119 |
| ETV6 (ets variant 6) | 2120 |
| ETV7 (ets variant 7) | 51513 |
| EVX1 (even-skipped homeobox 1) | 2128 |
| EVX2 (even-skipped homeobox 2) | 344191 |
| FEV (FEV (ETS oncogene family)) | 54738 |
| FLI1 (Friend leukemia virus integration 1) | 2313 |
| FLNA (filamin A, alpha (actin binding protein 280)) | 2316 |
| FOS (v-fos FBJ murine osteosarcoma viral oncogene homolog) | 2353 |
| FOSB (FBJ murine osteosarcoma viral oncogene homolog B) | 2354 |
| FOSL1 (FOS-like antigen 1) | 8061 |
| FOSL2 (FOS-like antigen 2) | 2355 |
| FOXA1 (forkhead box A1) | 3169 |
| FOXA2 (forkhead box A2 factor-3-beta; hepatocyte nuclear factor 3, beta) | 3170 |
| FOXA3 (forkhead box A3) | 3171 |
| FOXB1 (forkhead box B1) | 27023 |
| FOXB2 (forkhead box B2) | 442425 |
| FOXC1 (forkhead box C1) | 2296 |
| FOXC2 (forkhead box C2 (MFH-1, mesenchyme forkhead 1)) | 2303 |
| FOXD1 (forkhead box D1) | 2297 |
| FOXD2 (forkhead box D2) | 2306 |
| FOXD3 (forkhead box D3) | 27022 |
| FOXD4 (forkhead box D4) | 2298 |
| FOXD4L1 (forkhead box D4-like 1) | 200350 |
| FOXD4L3 (forkhead box D4-like 3) | 286380 |
| FOXD4L4 (forkhead box D4-like 4) | 349334 |
| FOXD4L5 (forkhead box D4-like 5) | 653427 |
| FOXD4L6 (forkhead box D4-like 6) | 653404 |
| FOXE1 (forkhead box E1 (thyroid transcription factor 2)) | 2304 |
| FOXE3 (forkhead box E3) | 2301 |
| FOXF1 (forkhead box F1) | 2294 |
| FOXF2 (forkhead box F2) | 2295 |
| FOXG1 (forkhead box G1) | 2290 |
| FOXH1 (forkhead box H1) | 8928 |
| FOXI1 (forkhead box I1) | 2299 |
| FOXI2 (forkhead box I2) | 399823 |
| FOXI3 (forkhead box I3) | 344167 |
| FOXJ1 (forkhead box J1) | 2302 |
| FOXJ2 (forkhead box J2) | 55810 |
| FOXJ3 (forkhead box J3) | 22887 |
| FOXK1 (forkhead box K1) | 221937 |
| FOXK2 (forkhead box K2) | 3607 |
| FOXL1 (forkhead box L1) | 2300 |
| FOXL2 (forkhead box L2) | 668 |
| FOXM1 (forkhead box M1) | 2305 |
| FOXN1 (forkhead box N1) | 8456 |
| FOXN2 (forkhead box N2) | 3344 |
| FOXN3 (forkhead box N3) | 1112 |
| FOXN4 (forkhead box N4) | 121643 |
| FOXO1 (forkhead box O1) | 2308 |
| FOXO3 (forkhead box O3) | 2309 |
| FOXO4 (forkhead box O4) | 4303 |
| FOXO6 (forkhead box protein O6) | 100132074 |
| FOXP1 (forkhead box P1) | 27086 |
| FOXP2 (forkhead box P2) | 93986 |
| FOXP3 (forkhead box P3) | 50943 |
| FOXP4 (forkhead box P4) | 116113 |
| FOXQ1 (forkhead box Q1) | 94234 |
| FOXR1 (forkhead box R1) | 283150 |
| FOXR2 (forkhead box R2) | 139628 |
| FOXS1 (forkhead box S1) | 2307 |
| FUBP1 (far upstream element (FUSE) binding protein 1)) | 8880 |
| GABPA (GA binding protein transcription factor, alpha subunit 60 kDa) | 2551 |
| GABPB1 (GA binding protein transcription factor, beta subunit 1) | 2553 |
| GAS7 (growth arrest-specific 7) | 8522 |
| GATA1 (GATA binding protein 1 (globin transcription factor 1)) | 2623 |
| GATA2 (GATA binding protein 2) | 2624 |
| GATA3 (GATA binding protein 3) | 2625 |
| GATA4 (GATA binding protein 4) | 2626 |
| GATA5 (GATA binding protein 5) | 140628 |
| GATA6 (GATA binding protein 6) | 2627 |
| GATAD1 (GATA zinc finger domain containing 1) | 57798 |
| GATAD2A (GATA zinc finger domain containing 2A) | 54815 |
| GATAD2B (GATA zinc finger domain containing 2B) | 57459 |
| GBX1 (gastrulation brain homeobox 1) | 2636 |
| GBX2 (gastrulation brain homeobox 2) | 2637 |
| GCM1 (glial cells missing homolog 1 (Drosophila)) | 8521 |
| GFI1B (growth factor independent 1B transcription repressor) | 8328 |
| GLI2 (GLI family zinc finger 2) | 2736 |
| GLI3 (GLI family zinc finger 3) | 2737 |
| GLIS1 (GLIS family zinc finger 1) | 148979 |
| GLIS3 (GLIS family zinc finger 3) | 169792 |
| GATA like protein-1) | 100125288 |
| GMEB2 (glucocorticoid modulatory element binding protein 2) | 26205 |
| GSC (goosecoid homeobox) | 145258 |
| GSC2 (goosecoid homeobox 2) | 2928 |
| GSX1 (GS homeobox 1) | 219409 |
| GSX2 (GS homeobox 2) | 170825 |
| GTF2A1 (general transcription factor IIA, 1, 19/37 kDa) | 2957 |
| GTF2A1L (general transcription factor IIA, 1-like) | 11036 |
| GTF2A2 (general transcription factor IIA, 2, 12 kDa) | 2958 |
| GTF2B (general transcription factor IIB) | 2959 |
| GTF2E1 (general transcription factor IIE, polypeptide 1, alpha 56 kDa) | 2960 |
| GTF2E2 (general transcription factor IIE, polypeptide 2, beta 34 kDa) | 2961 |
| GTF2F1 (general transcription factor IIF, polypeptide 1, 74 kDa) | 2962 |
| GTF2F2 (general transcription factor IIF, polypeptide 2, 30 kDa) | 2963 |
| GTF2H1 (general transcription factor IIH, polypeptide 1, 62 kDa) | 2965 |
| GTF2H2 (general transcription factor IIH, polypeptide 2, 44 kDa) | 2966 |
| GTF2H3 (general transcription factor IIH, polypeptide 3, 34 kDa) | 2967 |
| GTF2H4 (general transcription factor IIH, polypeptide 4, 52 kDa) | 2968 |
| GTF2I (general transcription factor II, i) | 2969 |
| GTF2IRD1 (GTF2I repeat domain containing 1) | 9569 |
| GTF3A (general transcription factor IIIA) | 2971 |
| GTF3C1 (general transcription factor IIIC, polypeptide 1, alpha 220 kDa) | 2975 |
| GTF3C2 (general transcription factor IIIC, polypeptide 2, beta 110 kDa) | 2976 |
| GTF3C3 (general transcription factor IIIC, polypeptide 3, 102 kDa) | 9330 |
| GTF3C4 (general transcription factor IIIC, polypeptide 4, 90 kDa) | 9329 |
| GTF3C5 (general transcription factor IIIC, polypeptide 5, 63 kDa) | 9328 |
| GTF3C6 (general transcription factor IIIC, polypeptide 6, alpha 35 kDa) | 112495 |
| HAND1 (heart and neural crest derivatives expressed 1) | 9421 |
| HAND2 (heart and neural crest derivatives expressed 2) | 9464 |
| HCFC1 (host cell factor C1 (VP16-accessory protein)) | 3054 |
| HCFC2 (host cell factor C2) | 29915 |
| HCLS1 (hematopoietic cell-specific Lyn substrate 1) | 3059 |
| HDAC1 (histone deacetylase 1) | 3065 |
| HDAC2 (histone deacetylase 2) | 3066 |
| HDX (highly divergent homeobox) | 139324 |
| HELT (HES/HEY-like transcription factor) | 391723 |
| HES6 (hairy and enhancer of split 6 (Drosophila)) | 55502 |
| HESX1 (HESX homeobox 1) | 8820 |
| HEY1 (hairy/enhancer-of-split related with YRPW motif 1) | 23462 |
| HEY2 (hairy/enhancer-of-split related with YRPW motif 2) | 23493 |
| HEYL (hairy/enhancer-of-split related with YRPW motif-like) | 26508 |
| HHEX (hematopoietically expressed homeobox) | 3087 |
| HIC1 (hypermethylated in cancer 1) | 3090 |
| HIF1A (hypoxia inducible factor 1, alpha subunit (basic helix-loop-helix transcription | 3091 |
| factor)) | |
| HIRA (HIR histone cell cycle regulation defective homolog A (S. cerevisiae)) | 7290 |
| HLF (hepatic leukemia factor) | 3131 |
| HLTF (helicase-like transcription factor) | 6596 |
| HLX (H2.0-like homeobox) | 3142 |
| HMBOX1 (homeobox containing 1) | 79618 |
| HMG20A (high-mobility group 20A) | 10363 |
| HMG20B (high-mobility group 20B) | 10362 |
| HMGA1 (high mobility group AT-hook 1) | 3159 |
| HMGB2 (high-mobility group box 2) | 3148 |
| HMGN1 (high-mobility group nucleosome binding domain 1) | 3150 |
| HMOX1 (heme oxygenase (decycling) 1) | 3162 |
| HMX1 (H6 family homeobox 1) | 3166 |
| HMX2 (H6 family homeobox 2) | 3167 |
| HMX3 (H6 family homeobox 3) | 340784 |
| HNF1A (HNF1 homeobox A) | 6927 |
| HNF1B (HNF1 homeobox B) | 6928 |
| HNF4A (hepatocyte nuclear factor 4, alpha) | 3172 |
| HNF4G (hepatocyte nuclear factor 4, gamma) | 3174 |
| HNRNPAB (heterogeneous nuclear ribonucleoprotein A/B) | 3182 |
| HOMEZ (homeobox and leucine zipper encoding) | 57594 |
| HOPX (HOP homeobox) | 84525 |
| HOXA1 (homeobox A1) | 3198 |
| HOXA10 (homeobox A10) | 3206 |
| HOXA11 (homeobox A11) | 3207 |
| HOXA13 (homeobox A13) | 3209 |
| HOXA2 (homeobox A2) | 3199 |
| HOXA3 (homeobox A3) | 3200 |
| HOXA4 (homeobox A4) | 3201 |
| HOXA5 (homeobox A5) | 3202 |
| HOXA6 (homeobox A6) | 3203 |
| HOXA7 (homeobox A7) | 3204 |
| HOXA9 (homeobox A9) | 3205 |
| HOXB1 (homeobox B1) | 3211 |
| HOXB13 (homeobox B13) | 10481 |
| HOXB2 (homeobox B2) | 3212 |
| HOXB3 (homeobox B3) | 3213 |
| HOXB4 (homeobox B4) | 3214 |
| HOXB5 (homeobox B5) | 3215 |
| HOXB6 (homeobox B6) | 3216 |
| HOXB7 (homeobox B7) | 3217 |
| HOXB8 (homeobox B8) | 3218 |
| HOXB9 (homeobox B9) | 3219 |
| HOXC10 (homeobox C10) | 3226 |
| HOXC11 (homeobox C11) | 3227 |
| HOXC12 (homeobox C12) | 3228 |
| HOXC13 (homeobox C13) | 3229 |
| HOXC4 (homeobox C4) | 3221 |
| HOXC5 (homeobox C5) | 3222 |
| HOXC6 (homeobox C6) | 3223 |
| HOXC8 (homeobox C8) | 3224 |
| HOXC9 (homeobox C9) | 3225 |
| HOXD1 (homeobox D1) | 3231 |
| HOXD10 (homeobox D10) | 3236 |
| HOXD11 (homeobox D11) | 3237 |
| HOXD12 (homeobox D12) | 3238 |
| HOXD13 (homeobox D13) | 3239 |
| HOXD3 (homeobox D3) | 3232 |
| HOXD4 (homeobox D4) | 3233 |
| HOXD8 (homeobox D8) | 3234 |
| HOXD9 (homeobox D9) | 3235 |
| HR (hairless homolog (mouse)) | 55806 |
| HSF1 (heat shock transcription factor 1) | 3297 |
| HSF2 (heat shock transcription factor 2) | 3298 |
| HSF4 (heat shock transcription factor 4) | 3299 |
| HSF5 (heat shock transcription factor family member 5) | 124535 |
| HSFX2 (heat shock transcription factor family, X linked 2) | 100130086 |
| HSFY2 (heat shock transcription factor, Y linked 2) | 159119 |
| HTATIP2 (HIV-1 Tat interactive protein 2, 30 kDa) | 10553 |
| HTATSF1 (HIV-1 Tat specific factor 1) | 27336 |
| ID1 (inhibitor of DNA binding 1, dominant negative helix-loop-helix protein) | 3397 |
| ID2 (inhibitor of DNA binding 2, dominant negative helix-loop-helix protein) | 3398 |
| ID3 (inhibitor of DNA binding 3, dominant negative helix-loop-helix protein) | 3399 |
| IKBKB (inhibitor of kappa light polypeptide gene enhancer in B-cells, kinase beta) | 3551 |
| IKZF3 (IKAROS family zinc finger 3 (Aiolos)) | 22806 |
| IKZF4 (IKAROS family zinc finger 4 (Eos)) | 64375 |
| IL1B (interleukin 1, beta) | 3553 |
| IL6 (interleukin 6 (interferon, beta 2)) | 3569 |
| ILF2 (interleukin enhancer binding factor 2, 45 kDa) | 3608 |
| IRAK2 (interleukin-1 receptor-associated kinase 2) | 3656 |
| IRF1 (interferon regulatory factor 1) | 3659 |
| IRF2 (interferon regulatory factor 2) | 3660 |
| IRF3 (interferon regulatory factor 3) | 3661 |
| IRF4 (interferon regulatory factor 4) | 3662 |
| IRF5 (interferon regulatory factor 5) | 3663 |
| IRF6 (interferon regulatory factor 6) | 3664 |
| IRF7 (interferon regulatory factor 7) | 3665 |
| IRF8 (interferon regulatory factor 8) | 3394 |
| IRF9 (interferon regulatory factor 9) | 10379 |
| IRX1 (iroquois homeobox 1) | 79192 |
| IRX2 (iroquois homeobox 2) | 153572 |
| IRX3 (iroquois homeobox 3) | 79191 |
| IRX4 (iroquois homeobox 4) | 50805 |
| IRX5 (iroquois homeobox 5) | 10265 |
| IRX6 (iroquois homeobox 6) | 79190 |
| ISL1 (ISL LIM homeobox 1) | 3670 |
| ISL2 (ISL LIM homeobox 2) | 64843 |
| ISX (intestine-specific homeobox) | 91464 |
| ITGB2 (integrin, beta 2 (complement component 3 receptor 3 and 4 subunit)) | 3689 |
| JDP2 (Jun dimerization protein 2) | 122953 |
| JMY (junction mediating and regulatory protein, p53 cofactor) | 133746 |
| JUN (jun oncogene) | 3725 |
| JUNB (jun B proto-oncogene) | 3726 |
| JUND (jun D proto-oncogene) | 3727 |
| KDM1 (lysine (K)-specific demethylase 1) | 23028 |
| KDM5A (lysine (K)-specific demethylase 5A) | 5927 |
| KDM5B (lysine (K)-specific demethylase 5B) | 10765 |
| KLF1 (Kruppel-like factor 1 (erythroid)) | 10661 |
| KLF10 (Kruppel-like factor 10) | 7071 |
| KLF11 (Kruppel-like factor 11) | 8462 |
| KLF12 (Kruppel-like factor 12) | 11278 |
| KLF13 (Kruppel-like factor 13) | 51621 |
| KLF15 (Kruppel-like factor 15) | 28999 |
| KLF16 (Kruppel-like factor 16) | 83855 |
| KLF2 (Kruppel-like factor 2 (lung)) | 10365 |
| KLF3 (Kruppel-like factor 3 (basic)) | 51274 |
| KLF4 (Kruppel-like factor 4 (gut)) | 9314 |
| KLF5 (Kruppel-like factor 5 (intestinal)) | 688 |
| KLF7 (Kruppel-like factor 7 (ubiquitous)) | 8609 |
| KLF9 (Kruppel-like factor 9) | 687 |
| L3MBTL (l(3)mbt-like (Drosophila)) | 26013 |
| L3MBTL4 (l(3)mbt-like 4 (Drosophila)) | 91133 |
| LASS2 (LAG1 homolog, ceramide synthase 2) | 29956 |
| LASS3 (LAG1 homolog, ceramide synthase 3) | 204219 |
| LASS4 (LAG1 homolog, ceramide synthase 4) | 79603 |
| LASS5 (LAG1 homolog, ceramide synthase 5) | 91012 |
| LASS6 (LAG1 homolog, ceramide synthase 6) | 253782 |
| LBX1 (ladybird homeobox 1) | 10660 |
| LBX2 (ladybird homeobox 2) | 85474 |
| LCOR (ligand dependent nuclear receptor corepressor) | 84458 |
| LCORL (ligand dependent nuclear receptor corepressor-like) | 254251 |
| LEF1 (lymphoid enhancer-binding factor 1) | 51176 |
| LHX1 (LIM homeobox 1) | 3975 |
| LHX2 (LIM homeobox 2) | 9355 |
| LHX3 (LIM homeobox 3) | 8022 |
| LHX4 (LIM homeobox 4) | 89884 |
| LHX5 (LIM homeobox 5) | 64211 |
| LHX6 (LIM homeobox 6) | 26468 |
| LHX8 (LIM homeobox 8) | 431707 |
| LHX9 (LIM homeobox 9) | 56956 |
| LITAF (lipopolysaccharide-induced TNF factor) | 9516 |
| LMO1 (LIM domain only 1 (rhombotin 1)) | 4004 |
| LMO4 (LIM domain only 4) | 8543 |
| LMX1A (LIM homeobox transcription factor 1, alpha) | 4009 |
| LMX1B (LIM homeobox transcription factor 1, beta) | 4010 |
| TBP-associated factor 11 pseudogene) | 391742 |
| LZTR1 (leucine-zipper-like transcription regulator 1) | 8216 |
| LZTS1 (leucine zipper, putative tumor suppressor 1) | 11178 |
| MAF (v-maf musculoaponeurotic fibrosarcoma oncogene homolog (avian)) | 4094 |
| MAFA (v-maf musculoaponeurotic fibrosarcoma oncogene homolog A (avian)) | 389692 |
| MAFB (v-maf musculoaponeurotic fibrosarcoma oncogene homolog B (avian)) | 9935 |
| MAFF (v-maf musculoaponeurotic fibrosarcoma oncogene homolog F (avian)) | 23764 |
| MAFG (v-maf musculoaponeurotic fibrosarcoma oncogene homolog G (avian)) | 4097 |
| MAFK (v-maf musculoaponeurotic fibrosarcoma oncogene homolog K (avian)) | 7975 |
| MAP3K13 (mitogen-activated protein kinase kinase kinase 13) | 9175 |
| MAX (MYC associated factor X) | 4149 |
| MBD1 (methyl-CpG binding domain protein 1) | 4152 |
| MDS1 (myelodysplasia syndrome 1) | 4197 |
| MED20 (mediator complex subunit 20) | 9477 |
| MED21 (mediator complex subunit 21) | 9412 |
| MED6 (mediator complex subunit 6) | 10001 |
| MEF2A (myocyte enhancer factor 2A) | 4205 |
| MEF2B (myocyte enhancer factor 2B) | 100271849 |
| MEF2C (myocyte enhancer factor 2C) | 4208 |
| MEF2D (myocyte enhancer factor 2D) | 4209 |
| MEIS1 (Meis homeobox 1) | 4211 |
| MEIS2 (Meis homeobox 2) | 4212 |
| MEIS3 (Meis homeobox 3) | 56917 |
| MEIS3P1 (Meis homeobox 3 pseudogene 1) | 4213 |
| MEIS3P2 (Meis homeobox 3 pseudogene 2) | 257468 |
| MEN1 (multiple endocrine neoplasia I) | 4221 |
| MEOX1 (mesenchyme homeobox 1) | 4222 |
| MEOX2 (mesenchyme homeobox 2) | 4223 |
| MESP2 (mesoderm posterior 2 homolog (mouse)) | 145873 |
| MGA (MAX gene associated) | 23269 |
| MIXL1 (Mix1 homeobox-like 1 (Xenopus laevis)) | 83881 |
| MKX (mohawk homeobox) | 283078 |
| MLL (myeloid/lymphoid or mixed-lineage leukemia (trithorax homolog, Drosophila)) | 4297 |
| myeloid/lymphoid or mixed-lineage leukemia 4) | 9757 |
| MLLT1 (myeloid/lymphoid or mixed-lineage leukemia (trithorax homolog, Drosophila)) | 4298 |
| MLLT10 (myeloid/lymphoid or mixed-lineage leukemia (trithorax homolog, Drosophila)) | 8028 |
| MLX (MAX-like protein X) | 6945 |
| MLXIPL (MLX interacting protein-like) | 51085 |
| MNT (MAX binding protein) | 4335 |
| MNX1 (motor neuron and pancreas homeobox 1) | 3110 |
| MRPL28 (mitochondrial ribosomal protein L28) | 10573 |
| MSC (musculin (activated B-cell factor-1)) | 9242 |
| MSL3 (male-specific lethal 3 homolog (Drosophila)) | 10943 |
| MSRB2 (methionine sulfoxide reductase B2) | 22921 |
| MSX1 (msh homeobox 1) | 4487 |
| MSX2 (msh homeobox 2) | 4488 |
| MTA1 (metastasis associated 1) | 9112 |
| MTA2 (metastasis associated 1 family, member 2) | 9219 |
| MTA3 (metastasis associated 1 family, member 3) | 57504 |
| MTDH (metadherin) | 92140 |
| MTF1 (metal-regulatory transcription factor 1) | 4520 |
| MXD1 (MAX dimerization protein 1) | 4084 |
| MYBL2 (v-myb myeloblastosis viral oncogene homolog (avian)-like 2) | 4605 |
| MYC (v-myc myelocytomatosis viral oncogene homolog (avian)) | 4609 |
| MYCL1 (v-myc myelocytomatosis viral oncogene homolog 1, lung carcinoma derived | 4610 |
| (avian)) | |
| MYCN (v-myc myelocytomatosis viral related oncogene, neuroblastoma derived (avian)) | 4613 |
| MYD88 (myeloid differentiation primary response gene (88)) | 4615 |
| MYF5 (myogenic factor 5) | 4617 |
| MYF6 (myogenic factor 6 (herculin)) | 4618 |
| MYNN (myoneurin) | 55892 |
| MYOD1 (myogenic differentiation 1) | 4654 |
| MYOG (myogenin (myogenic factor 4)) | 4656 |
| MYPOP (Myb-related transcription factor, partner of profilin) | 339344 |
| MYST2 (MYST histone acetyltransferase 2) | 11143 |
| MYT1 (myelin transcription factor 1) | 4661 |
| MYT1L (myelin transcription factor 1-like) | 23040 |
| MZF1 (myeloid zinc finger 1) | 7593 |
| NANOG (Nanog homeobox) | 79923 |
| NANOGP1 (Nanog homeobox pseudogene 1) | 404635 |
| NANOGP8 (Nanog homeobox pseudogene 8) | 388112 |
| NARFL (nuclear prelamin A recognition factor-like) | 64428 |
| NCOR1 (nuclear receptor co-repressor 1) | 9611 |
| NEUROD1 (neurogenic differentiation 1) | 4760 |
| NEUROD2 (neurogenic differentiation 2) | 4761 |
| NEUROG1 (neurogenin 1) | 4762 |
| NEUROG3 (neurogenin 3) | 50674 |
| NFAM1 (NFAT activating protein with ITAM motif 1) | 150372 |
| NFAT5 (nuclear factor of activated T-cells 5, tonicity-responsive) | 10725 |
| NFATC1 (nuclear factor of activated T-cells, cytoplasmic, calcineurin-dependent 1) | 4772 |
| NFATC2 (nuclear factor of activated T-cells, cytoplasmic, calcineurin-dependent 2) | 4773 |
| NFATC3 (nuclear factor of activated T-cells, cytoplasmic, calcineurin-dependent 3) | 4775 |
| NFATC4 (nuclear factor of activated T-cells, cytoplasmic, calcineurin-dependent 4) | 4776 |
| NFE2 (nuclear factor (erythroid-derived 2), 45 kDa) | 4778 |
| NFE2L1 (nuclear factor (erythroid-derived 2)-like 1) | 4779 |
| NFE2L2 (nuclear factor (erythroid-derived 2)-like 2) | 4780 |
| NFE2L3 (nuclear factor (erythroid-derived 2)-like 3) | 9603 |
| NFIA (nuclear factor I/A) | 4774 |
| NFIB (nuclear factor I/B) | 4781 |
| NFIC (nuclear factor I/C (CCAAT-binding transcription factor)) | 4782 |
| NFIL3 (nuclear factor, interleukin 3 regulated) | 4783 |
| NFIX (nuclear factor I/X (CCAAT-binding transcription factor)) | 4784 |
| NFKB1 (nuclear factor of kappa light polypeptide gene enhancer in B-cells 1) | 4790 |
| NFKB2 (nuclear factor of kappa light polypeptide gene enhancer in B-cells 2 (p49/p100)) | 4791 |
| NFRKB (nuclear factor related to kappaB binding protein) | 4798 |
| NFX1 (nuclear transcription factor, X-box binding 1) | 4799 |
| NFXL1 (nuclear transcription factor, X-box binding-like 1) | 152518 |
| NFYA (nuclear transcription factor Y, alpha) | 4800 |
| NFYB (nuclear transcription factor Y, beta) | 4801 |
| NFYC (nuclear transcription factor Y, gamma) | 4802 |
| NKX1-1 (NK1 homeobox 1) | 54729 |
| NKX1-2 (NK1 homeobox 2) | 390010 |
| NKX2-1 (NK2 homeobox 1) | 7080 |
| NKX2-2 (NK2 homeobox 2) | 4821 |
| NKX2-3 (NK2 transcription factor related, locus 3 (Drosophila)) | 159296 |
| NKX2-4 (NK2 homeobox 4) | 644524 |
| NKX2-5 (NK2 transcription factor related, locus 5 (Drosophila)) | 1482 |
| NKX2-6 (NK2 transcription factor related, locus 6 (Drosophila)) | 137814 |
| NKX2-8 (NK2 homeobox 8) | 26257 |
| NKX3-1 (NK3 homeobox 1 transcription factor related, locus 1) | 4824 |
| NKX3-2 (NK3 homeobox 2) | 579 |
| NKX6-1 (NK6 homeobox 1) | 4825 |
| NKX6-2 (NK6 homeobox 2) | 84504 |
| NKX6-3 (NK6 homeobox 3) | 157848 |
| NLRC3 (NLR family, CARD domain containing 3) | 197358 |
| NLRP3 (NLR family, pyrin domain containing 3) | 114548 |
| NME2 (non-metastatic cells 2) | 4831 |
| NOBOX (NOBOX oogenesis homeobox) | 135935 |
| NOD2 (nucleotide-binding oligomerization domain containing 2) | 64127 |
| NOTCH1 (Notch homolog 1, translocation-associated (Drosophila)) | 4851 |
| NOTCH2 (Notch homolog 2 (Drosophila)) | 4853 |
| NOTO (notochord homeobox) | 344022 |
| NPAS1 (neuronal PAS domain protein 1) | 4861 |
| NPAS2 (neuronal PAS domain protein 2) | 4862 |
| NPM1 (nucleophosmin (nucleolar phosphoprotein B23, numatrin)) | 4869 |
| NR0B1 (nuclear receptor subfamily 0, group B, member 1) | 190 |
| NR0B2 (nuclear receptor subfamily 0, group B, member 2) | 8431 |
| NR1D1 (nuclear receptor subfamily 1, group D, member 1) | 9572 |
| NR1D2 (nuclear receptor subfamily 1, group D, member 2) | 9975 |
| NR1H2 (nuclear receptor subfamily 1, group H, member 2) | 7376 |
| NR1H3 (nuclear receptor subfamily 1, group H, member 3) | 10062 |
| NR1H4 (nuclear receptor subfamily 1, group H, member 4) | 9971 |
| NR1I2 (nuclear receptor subfamily 1, group I, member 2) | 8856 |
| NR1I3 (nuclear receptor subfamily 1, group I, member 3) | 9970 |
| NR2C1 (nuclear receptor subfamily 2, group C, member 1) | 7181 |
| NR2C2 (nuclear receptor subfamily 2, group C, member 2) | 7182 |
| NR2E1 (nuclear receptor subfamily 2, group E, member 1) | 7101 |
| NR2E3 (nuclear receptor subfamily 2, group E, member 3) | 10002 |
| NR2F1 (nuclear receptor subfamily 2, group F, member 1) | 7025 |
| NR2F2 (nuclear receptor subfamily 2, group F, member 2) | 7026 |
| NR2F6 (nuclear receptor subfamily 2, group F, member 6) | 2063 |
| NR3C1 (nuclear receptor subfamily 3, group C, member 1 (glucocorticoid receptor)) | 2908 |
| NR3C2 (nuclear receptor subfamily 3, group C, member 2) | 4306 |
| NR4A1 (nuclear receptor subfamily 4, group A, member 1) | 3164 |
| NR4A2 (nuclear receptor subfamily 4, group A, member 2) | 4929 |
| NR4A3 (nuclear receptor subfamily 4, group A, member 3) | 8013 |
| NR5A1 (nuclear receptor subfamily 5, group A, member 1) | 2516 |
| NR5A2 (nuclear receptor subfamily 5, group A, member 2) | 2494 |
| NR6A1 (nuclear receptor subfamily 6, group A, member 1) | 2649 |
| NRK (Nik related kinase) | 203447 |
| NRL (neural retina leucine zipper) | 4901 |
| OLIG2 (oligodendrocyte lineage transcription factor 2) | 10215 |
| ONECUT1 (one cut homeobox 1) | 3175 |
| ONECUT2 (one cut homeobox 2) | 9480 |
| ONECUT3 (one cut homeobox 3) | 390874 |
| OTP (orthopedia homeobox) | 23440 |
| OTX1 (orthodenticle homeobox 1) | 5013 |
| OTX2 (orthodenticle homeobox 2) | 5015 |
| PA2G4 (proliferation-associated 2G4, 38 kDa) | 5036 |
| PAX3 (paired box 3) | 5077 |
| PAX4 (paired box 4) | 5078 |
| PAX6 (paired box 6) | 5080 |
| PAX7 (paired box 7) | 5081 |
| PAX8 (paired box 8) | 7849 |
| PBX1 (pre-B-cell leukemia homeobox 1) | 5087 |
| PBX2 (pre-B-cell leukemia homeobox 2) | 5089 |
| PBX3 (pre-B-cell leukemia homeobox 3) | 5090 |
| PBX4 (pre-B-cell leukemia homeobox 4) | 80714 |
| PCGF2 (polycomb group ring finger 2) | 7703 |
| PCGF6 (polycomb group ring finger 6) | 84108 |
| PDX1 (pancreatic and duodenal homeobox 1) | 3651 |
| PEG3 (paternally expressed 3) | 5178 |
| PEX14 (peroxisomal biogenesis factor 14) | 5195 |
| PFDN1 (prefoldin subunit 1) | 5201 |
| PGBD1 (piggyBac transposable element derived 1) | 84547 |
| PGR (progesterone receptor) | 5241 |
| PHF1 (PHD finger protein 1) | 5252 |
| PHF5A (PHD finger protein 5A) | 84844 |
| PHOX2A (paired-like homeobox 2a) | 401 |
| PHOX2B (paired-like homeobox 2b) | 8929 |
| PHTF1 (putative homeodomain transcription factor 1) | 10745 |
| PITX1 (paired-like homeodomain 1) | 5307 |
| PITX2 (paired-like homeodomain 2) | 5308 |
| PITX3 (paired-like homeodomain 3) | 5309 |
| PKNOX1 (PBX/knotted 1 homeobox 1) | 5316 |
| PKNOX2 (PBX/knotted 1 homeobox 2) | 63876 |
| PLA2G1B (phospholipase A2, group IB (pancreas)) | 5319 |
| PLAG1 (pleiomorphic adenoma gene 1) | 5324 |
| PLAGL2 (pleiomorphic adenoma gene-like 2) | 5326 |
| POU1F1 (POU class 1 homeobox 1) | 5449 |
| POU2F1 (POU class 2 homeobox 1) | 5451 |
| POU2F2 (POU class 2 homeobox 2) | 5452 |
| POU2F3 (POU class 2 homeobox 3) | 25833 |
| POU3F1 (POU class 3 homeobox 1) | 5453 |
| POU3F2 (POU class 3 homeobox 2) | 5454 |
| POU3F3 (POU class 3 homeobox 3) | 5455 |
| POU3F4 (POU class 3 homeobox 4) | 5456 |
| POU4F1 (POU class 4 homeobox 1) | 5457 |
| POU4F2 (POU class 4 homeobox 2) | 5458 |
| POU4F3 (POU class 4 homeobox 3) | 5459 |
| POU5F1 (POU class 5 homeobox 1) | 5460 |
| POU5F1B (POU class 5 homeobox 1B) | 5462 |
| POU5F2 (POU domain class 5, transcription factor 2) | 134187 |
| POU6F1 (POU class 6 homeobox 1) | 5463 |
| POU6F2 (POU class 6 homeobox 2) | 11281 |
| PPARA (peroxisome proliferator-activated receptor alpha) | 5465 |
| PPARD (peroxisome proliferator-activated receptor delta) | 5467 |
| PPARG (peroxisome proliferator-activated receptor gamma) | 5468 |
| PRDM1 (PR domain containing 1, with ZNF domain) | 639 |
| PRDM16 (PR domain containing 16) | 63976 |
| PRDM2 (PR domain containing 2, with ZNF domain) | 7799 |
| PRDM4 (PR domain containing 4) | 11108 |
| PRDX3 (peroxiredoxin 3) | 10935 |
| PROP1 (PROP paired-like homeobox 1) | 5626 |
| PROX1 (prospero homeobox 1) | 5629 |
| PRRX1 (paired related homeobox 1) | 5396 |
| PRRX2 (paired related homeobox 2) | 51450 |
| PTTG1 (pituitary tumor-transforming 1) | 9232 |
| PURA (purine-rich element binding protein A) | 5813 |
| PURB (purine-rich element binding protein B) | 5814 |
| PYCARD (PYD and CARD domain containing) | 29108 |
| PYDC1 (PYD (pyrin domain) containing 1) | 260434 |
| RARA (retinoic acid receptor, alpha) | 5914 |
| RARB (retinoic acid receptor, beta) | 5915 |
| RARG (retinoic acid receptor, gamma) | 5916 |
| RAX (retina and anterior neural fold homeobox) | 30062 |
| RAX2 (retina and anterior neural fold homeobox 2) | 84839 |
| RB1 (retinoblastoma 1) | 5925 |
| RBPJ (recombination signal binding protein for immunoglobulin kappa J region) | 3516 |
| RBPJL (recombination signal binding protein for immunoglobulin kappa J region-like) | 11317 |
| RCAN1 (regulator of calcineurin 1) | 1827 |
| RCOR2 (REST corepressor 2) | 283248 |
| REL (v-rel reticuloendotheliosis viral oncogene homolog (avian)) | 5966 |
| RELA (v-rel reticuloendotheliosis viral oncogene homolog A (avian)) | 5970 |
| RELB (v-rel reticuloendotheliosis viral oncogene homolog B) | 5971 |
| RERE (arginine-glutamic acid dipeptide (RE) repeats) | 473 |
| REXO4 (REX4, RNA exonuclease 4 homolog (S. cerevisiae)) | 57109 |
| RFX1 (regulatory factor X, 1 (influences HLA class II expression)) | 5989 |
| RFX3 (regulatory factor X, 3 (influences HLA class II expression)) | 5991 |
| RFX5 (regulatory factor X, 5 (influences HLA class II expression)) | 5993 |
| RFXANK (regulatory factor X-associated ankyrin-containing protein) | 8625 |
| RFXAP (regulatory factor X-associated protein) | 5994 |
| RHOXF1 (Rhox homeobox family, member 1) | 158800 |
| RHOXF2 (Rhox homeobox family, member 2) | 84528 |
| RHOXF2B (Rhox homeobox family, member 2B) | 727940 |
| RIPK2 (receptor-interacting serine-threonine kinase 2) | 8767 |
| RLF (rearranged L-myc fusion) | 6018 |
| RNF4 (ring finger protein 4) | 6047 |
| RORA (RAR-related orphan receptor A) | 6095 |
| RORB (RAR-related orphan receptor B) | 6096 |
| RORC (RAR-related orphan receptor C) | 6097 |
| RPS3 (ribosomal protein S3) | 6188 |
| RUNX1 (runt-related transcription factor 1) | 861 |
| RUNX1T1 (runt-related transcription factor 1; translocated to, 1 (cyclin D-related)) | 862 |
| RUNX2 (runt-related transcription factor 2) | 860 |
| RUNX3 (runt-related transcription factor 3) | 864 |
| RXRA (retinoid X receptor, alpha) | 6256 |
| RXRB (retinoid X receptor, beta) | 6257 |
| RXRG (retinoid X receptor, gamma) | 6258 |
| SALL1 (sal-like 1 (Drosophila)) | 6299 |
| SALL2 (sal-like 2 (Drosophila)) | 6297 |
| SATB1 (SATB homeobox 1) | 6304 |
| SATB2 (SATB homeobox 2) | 23314 |
| SCAND1 (SCAN domain containing 1) | 51282 |
| SCAND2 (SCAN domain containing 2 pseudogene) | 54581 |
| SCAND3 (SCAN domain containing 3) | 114821 |
| SCMH1 (sex comb on midleg homolog 1 (Drosophila)) | 22955 |
| SCML1 (sex comb on midleg-like 1 (Drosophila)) | 6322 |
| SCML2 (sex comb on midleg-like 2 (Drosophila)) | 10389 |
| SCRT1 (scratch homolog 1, zinc finger protein (Drosophila)) | 83482 |
| SEBOX (SEBOX homeobox) | 645832 |
| SF1 (splicing factor 1) | 7536 |
| SHH (sonic hedgehog homolog (Drosophila)) | 6469 |
| SHOX (short stature homeobox) | 6473 |
| SHOX2 (short stature homeobox 2) | 6474 |
| SIGIRR (single immunoglobulin and toll-interleukin 1 receptor (TIR) domain) | 59307 |
| SIM1 (single-minded homolog 1 (Drosophila)) | 6492 |
| SIM2 (single-minded homolog 2 (Drosophila)) | 6493 |
| SIRT1 (sirtuin (silent mating type information regulation 2 homolog) 1 (S. cerevisiae)) | 23411 |
| SIX1 (SIX homeobox 1) | 6495 |
| SIX2 (SIX homeobox 2) | 10736 |
| SIX3 (SIX homeobox 3) | 6496 |
| SIX4 (SIX homeobox 4) | 51804 |
| SIX5 (SIX homeobox 5) | 147912 |
| SIX6 (SIX homeobox 6) | 4990 |
| SLC26A3 (solute carrier family 26, member 3) | 1811 |
| SLC2A4RG (SLC2A4 regulator) | 56731 |
| SLC30A9 (solute carrier family 30 (zinc transporter), member 9) | 10463 |
| SMAD1 (SMAD family member 1) | 4086 |
| SMAD2 (SMAD family member 2) | 4087 |
| SMAD3 (SMAD family member 3) | 4088 |
| SMAD4 (SMAD family member 4) | 4089 |
| SMAD5 (SMAD family member 5) | 4090 |
| SMAD6 (SMAD family member 6) | 4091 |
| SMAD7 (SMAD family member 7) | 4092 |
| SMAD9 (SMAD family member 9) | 4093 |
| SMARCA4 (SWI/SNF related, matrix associated, actin dependent regulator of) | 6597 |
| SMARCA5 (SWI/SNF related, matrix associated, actin dependent regulator of) | 8467 |
| SNAI3 (snail homolog 3 (Drosophila)) | 333929 |
| SNAPC2 (small nuclear RNA activating complex, polypeptide 2, 45 kDa) | 6618 |
| SNAPC4 (small nuclear RNA activating complex, polypeptide 4, 190 kDa) | 6621 |
| SNAPC5 (small nuclear RNA activating complex, polypeptide 5, 19 kDa) | 10302 |
| SNF8 (SNF8, ESCRT-II complex subunit, homolog (S. cerevisiae)) | 11267 |
| SOHLH1 (spermatogenesis and oogenesis specific basic helix-loop-helix 1) | 402381 |
| SOLH (small optic lobes homolog (Drosophila)) | 6650 |
| SOX1 (SRY (sex determining region Y)-box 1) | 6656 |
| SOX10 (SRY (sex determining region Y)-box 10) | 6663 |
| SOX12 (SRY (sex determining region Y)-box 12) | 6666 |
| SOX13 (SRY (sex determining region Y)-box 13) | 9580 |
| SOX14 (SRY (sex determining region Y)-box 14) | 8403 |
| SOX15 (SRY (sex determining region Y)-box 15) | 6665 |
| SOX18 (SRY (sex determining region Y)-box 18) | 54345 |
| SOX2 (SRY (sex determining region Y)-box 2) | 6657 |
| SOX21 (SRY (sex determining region Y)-box 21) | 11166 |
| SOX4 (SRY (sex determining region Y)-box 4) | 6659 |
| SOX5 (SRY (sex determining region Y)-box 5) | 6660 |
| SOX6 (SRY (sex determining region Y)-box 6) | 55553 |
| SOX7 (SRY (sex determining region Y)-box 7) | 83595 |
| SOX8 (SRY (sex determining region Y)-box 8) | 30812 |
| SOX9 (SRY (sex determining region Y)-box 9) | 6662 |
| SP1 (Sp1 transcription factor) | 6667 |
| SP100 (SP100 nuclear antigen) | 6672 |
| SP140 (SP140 nuclear body protein) | 11262 |
| SP2 (Sp2 transcription factor) | 6668 |
| SP4 (Sp4 transcription factor) | 6671 |
| SPDEF (SAM pointed domain containing ets transcription factor) | 25803 |
| SPEN (spen homolog, transcriptional regulator (Drosophila)) | 23013 |
| SPI1 (spleen focus forming virus (SFFV) proviral integration oncogene spi1) | 6688 |
| SPIB (Spi-B transcription factor (Spi-1/PU.1 related)) | 6689 |
| SPIC (Spi-C transcription factor (Spi-1/PU.1 related)) | 121599 |
| SREBF1 (sterol regulatory element binding transcription factor 1) | 6720 |
| SREBF2 (sterol regulatory element binding transcription factor 2) | 6721 |
| SRF (serum response factor (c-fos serum response element-binding transcription factor)) | 6722 |
| SRY (sex determining region Y) | 6736 |
| ST18 (suppression of tumorigenicity 18 (breast carcinoma) (zinc finger protein)) | 9705 |
| STAT1 (signal transducer and activator of transcription 1, 91 kDa) | 6772 |
| STAT2 (signal transducer and activator of transcription 2, 113 kDa) | 6773 |
| STAT3 (signal transducer and activator of transcription 3 (acute-phase response factor)) | 6774 |
| STAT4 (signal transducer and activator of transcription 4) | 6775 |
| STAT5A (signal transducer and activator of transcription 5A) | 6776 |
| STAT5B (signal transducer and activator of transcription 5B) | 6777 |
| STAT6 (signal transducer and activator of transcription 6) | 6778 |
| STK36 (serine/threonine kinase 36, fused homolog (Drosophila)) | 27148 |
| SUMO1 (SMT3 suppressor of mif two 3 homolog 1 (S. cerevisiae)) | 7341 |
| SUPT3H (suppressor of Ty 3 homolog (S. cerevisiae)) | 8464 |
| SUPT4H1 (suppressor of Ty 4 homolog 1 (S. cerevisiae)) | 6827 |
| SUPT6H (suppressor of Ty 6 homolog (S. cerevisiae)) | 6830 |
| transcription factor T) | 6862 |
| TADA2L (transcriptional adaptor 2 (ADA2 homolog, yeast)-like yeast, homolog)-like; | 6871 |
| transcriptional adaptor 2 alpha; transcriptional adaptor 2-like) | |
| TADA3L (transcriptional adaptor 3 (NGG1 homolog, yeast)-like) | 10474 |
| TAF10 (TAF10 RNA polymerase II, TATA box binding protein (TBP)-associated factor, | 6881 |
| 30 kDa) | |
| TAF11 (TAF11 RNA polymerase II, TATA box binding protein (TBP)-associated factor, | 6882 |
| 28 kDa) | |
| TAF12 (TAF12 RNA polymerase II, TATA box binding protein (TBP)-associated factor, | 6883 |
| 20 kDa) | |
| TAF13 (TAF13 RNA polymerase II, TATA box binding protein (TBP)-associated factor, | 6884 |
| 18 kDa) | |
| TAF1A (TATA box binding protein (TBP)-associated factor, RNA polymerase I, A, | 9015 |
| 48 kDa) | |
| TAF1B (TATA box binding protein (TBP)-associated factor, RNA polymerase I, B, | 9014 |
| 63 kDa) | |
| TAF1C (TATA box binding protein (TBP)-associated factor, RNA polymerase I, C, | 9013 |
| 110 kDa) | |
| TAF2 (TAF2 RNA polymerase II, TATA box binding protein (TBP)-associated factor, | 6873 |
| 150 kDa) | |
| TAF4 (TAF4 RNA polymerase II, TATA box binding protein (TBP)-associated factor, | 6874 |
| 135 kDa) | |
| TAF4B (TAF4b RNA polymerase II, TATA box binding protein (TBP)-associated factor, | 6875 |
| 105 kDa) | |
| TAF5 (TAF5 RNA polymerase II, TATA box binding protein (TBP)-associated factor, | 6877 |
| 100 kDa) | |
| TAF5L (TAF5-like RNA polymerase II, p300/CBP-associated factor (PCAF)-associated | 27097 |
| factor, 65 kDa) | |
| TAF6 (TAF6 RNA polymerase II, TATA box binding protein (TBP)-associated) | 6878 |
| TAF6L (TAF6-like RNA polymerase II, p300/CBP-associated factor (PCAF)-associated | 10629 |
| factor, 65 kDa) | |
| TAF7 (TAF7 RNA polymerase II, TATA box binding protein (TBP)-associated factor, | 6879 |
| 55 kDa) | |
| TAF7L (TAF7-like RNA polymerase II, TATA box binding protein (TBP)-associated | 54457 |
| factor, 50 kDa) | |
| TAF9 (TAF9 RNA polymerase II, TATA box binding protein (TBP)-associated factor, | 6880 |
| 32 kDa) | |
| TARDBP (TAR DNA binding protein) | 23435 |
| TBP (TATA box binding protein) | 6908 |
| TBPL1 (TBP-like 1) | 9519 |
| TBPL2 (TATA box binding protein like 2) | 387332 |
| TBR1 (T-box, brain, 1) | 10716 |
| TBX1 (T-box 1) | 6899 |
| TBX10 (T-box 10) | 347853 |
| TBX15 (T-box 15) | 6913 |
| TBX18 (T-box 18) | 9096 |
| TBX19 (T-box 19) | 9095 |
| TBX2 (T-box 2) | 6909 |
| TBX20 (T-box 20) | 57057 |
| TBX21 (T-box 21) | 30009 |
| TBX22 (T-box 22) | 50945 |
| TBX3 (T-box 3) | 6926 |
| TBX4 (T-box 4) | 9496 |
| TBX5 (T-box 5) | 6910 |
| TBX6 (T-box 6) | 6911 |
| TCEA1 (transcription elongation factor A (SII), 1) | 6917 |
| TCEA2 (transcription elongation factor A (SII), 2) | 6919 |
| TCEA3 (transcription elongation factor A (SII), 3) | 6920 |
| TCEAL1 (transcription elongation factor A (SII)-like 1) | 9338 |
| TCERG1 (transcription elongation regulator 1) | 10915 |
| TCF12 (transcription factor 12) | 6938 |
| TCF15 (transcription factor 15 (basic helix-loop-helix)) | 6939 |
| TCF19 (transcription factor 19) | 6941 |
| TCF25 (transcription factor 25 (basic helix-loop-helix)) | 22980 |
| TCF3 (transcription factor 3 (E2A immunoglobulin enhancer binding factors E12/E47)) | 6929 |
| TCF4 (transcription factor 4) | 6925 |
| TCF7 (transcription factor 7 (T-cell specific, HMG-box)) | 6932 |
| TCF7L1 (transcription factor 7-like 1 (T-cell specific, HMG-box)) | 83439 |
| TCF7L2 (transcription factor 7-like 2 (T-cell specific, HMG-box)) | 6934 |
| TCFL5 (transcription factor-like 5 (basic helix-loop-helix)) | 10732 |
| TEAD1 (TEA domain family member 1 (SV40 transcriptional enhancer factor)) | 7003 |
| TEAD2 (TEA domain family member 2) | 8463 |
| TEAD3 (TEA domain family member 3) | 7005 |
| TEAD4 (TEA domain family member 4) | 7004 |
| TEF (thyrotrophic embryonic factor) | 7008 |
| TFAM (transcription factor A, mitochondrial) | 7019 |
| TFAP2A (transcription factor AP-2 alpha (activating enhancer binding protein 2 alpha)) | 7020 |
| TFAP2B (transcription factor AP-2 beta (activating enhancer binding protein 2 beta)) | 7021 |
| TFAP2C (transcription factor AP-2 gamma (activating enhancer binding protein 2 | 7022 |
| gamma)) | |
| TFAP2D (transcription factor AP-2 delta (activating enhancer binding protein 2 delta)) | 83741 |
| TFAP2E (transcription factor AP-2 epsilon (activating enhancer binding protein 2 | 339488 |
| epsilon)) | |
| TFAP4 (transcription factor AP-4 (activating enhancer binding protein 4)) | 7023 |
| TFCP2 (transcription factor CP2) | 7024 |
| TFCP2L1 (transcription factor CP2-like 1) | 29842 |
| TFDP1 (transcription factor Dp-1) | 7027 |
| TFDP2 (transcription factor Dp-2 (E2F dimerization partner 2)) | 7029 |
| TFDP3 (transcription factor Dp family, member 3) | 51270 |
| TFE3 (transcription factor binding to IGHM enhancer 3) | 7030 |
| TFEB (transcription factor EB) | 7942 |
| TFEC (transcription factor EC) | 22797 |
| TGFB1 (transforming growth factor, beta 1) | 7040 |
| TGIF1 (TGFB-induced factor homeobox 1) | 7050 |
| TGIF2 (TGFB-induced factor homeobox 2) | 60436 |
| TGIF2LX (TGFB-induced factor homeobox 2-like, X-linked) | 90316 |
| TGIF2LY (TGFB-induced factor homeobox 2-like, Y-linked) | 90655 |
| THRA (thyroid hormone receptor, alpha (erythroblastic leukemia viral (v-erb-a) oncogene | 7067 |
| homolog, avian)) | |
| THRB (thyroid hormone receptor, beta (erythroblastic leukemia viral (v-erb-a) | 7068 |
| TIAL1 (TIA1 cytotoxic granule-associated RNA binding protein-like 1 protein; TIA-1 | 7073 |
| related protein; TIA-1-related nucleolysin; TIA1 cytotoxic granule-associated RNA- | |
| binding protein-like 1; aging-associated gene 7 protein) | |
| TLR3 (toll-like receptor 3) | 7098 |
| TLX1 (T-cell leukemia homeobox 1) | 3195 |
| TLX2 (T-cell leukemia homeobox 2) | 3196 |
| TLX3 (T-cell leukemia homeobox 3) | 30012 |
| TMF1 (TATA element modulatory factor 1) | 7110 |
| TNF (tumor necrosis factor (TNF superfamily, member 2)) | 7124 |
| TP53 (tumor protein p53) | 7157 |
| TP63 (tumor protein p63) | 8626 |
| TP73 (tumor protein p73) | 7161 |
| TPRX1 (tetra-peptide repeat homeobox 1) | 284355 |
| TRERF1 (transcriptional regulating factor 1) | 55809 |
| TRIB1 (tribbles homolog 1 (Drosophila) mitogenic pathways) | 10221 |
| TRIM22 (tripartite motif-containing 22) | 10346 |
| TRIM25 (tripartite motif-containing 25) | 7706 |
| TRIM28 (tripartite motif-containing 28) | 10155 |
| TRIM29 (tripartite motif-containing 29) | 23650 |
| TRPS1 (trichorhinophalangeal syndrome I) | 7227 |
| TSC22D1 (TSC22 domain family, member 1) | 8848 |
| TSC22D2 (TSC22 domain family, member 2) | 9819 |
| TSC22D3 (TSC22 domain family, member 3) | 1831 |
| TSC22D4 (TSC22 domain family, member 4) | 81628 |
| TSHZ1 (teashirt zinc finger homeobox 1) | 10194 |
| TSHZ2 (teashirt zinc finger homeobox 2) | 128553 |
| TSHZ3 (teashirt zinc finger homeobox 3) | 57616 |
| TSSK4 (testis-specific serine kinase 4) | 283629 |
| TULP4 (tubby like protein 4) | 56995 |
| UBE2N (ubiquitin-conjugating enzyme E2N (UBC13 homolog, yeast)) | 7334 |
| UBE2V1 (ubiquitin-conjugating enzyme E2 variant 1) | 7335 |
| UBN1 (ubinuclein 1) | 29855 |
| UBP1 (upstream binding protein 1 (LBP-1a)) | 7342 |
| UBTF (upstream binding transcription factor, RNA polymerase I) | 7343 |
| UHRF1 (ubiquitin-like with PHD and ring finger domains 1) | 29128 |
| UNCX (UNC homeobox) | 340260 |
| USF1 (upstream transcription factor 1) | 7391 |
| USF2 (upstream transcription factor 2, c-fos interacting) | 7392 |
| UTF1 (undifferentiated embryonic cell transcription factor 1) | 8433 |
| VAV1 (vav 1 guanine nucleotide exchange factor) | 7409 |
| VAX1 (ventral anterior homeobox 1) | 11023 |
| VAX2 (ventral anterior homeobox 2) | 25806 |
| VDR (vitamin D (1,25-dihydroxyvitamin D3) receptor) | 7421 |
| VENTX (VENT homeobox homolog (Xenopus laevis) hemopoietic progenitor homeobox | 27287 |
| protein VENTX2) | |
| VEZF1 (vascular endothelial zinc finger 1) | 7716 |
| VPS72 (vacuolar protein sorting 72 homolog (S. cerevisiae)) | 6944 |
| VSX1 (visual system homeobox 1) | 30813 |
| VSX2 (visual system homeobox 2) | 338917 |
| WT1 (Wilms tumor 1) | 7490 |
| XBP1 (X-box binding protein 1) | 7494 |
| YBX1 (Y box binding protein 1) | 4904 |
| YEATS4 (YEATS domain containing 4) | 8089 |
| YY1 (YY1 transcription factor) | 7528 |
| ZBTB17 (zinc finger and BTB domain containing 17) | 7709 |
| ZBTB25 (zinc finger and BTB domain containing 25) | 7597 |
| ZBTB32 (zinc finger and BTB domain containing 32) | 27033 |
| ZBTB38 (zinc finger and BTB domain containing 38) | 253461 |
| ZBTB48 (zinc finger and BTB domain containing 48) | 3104 |
| ZBTB7B (zinc finger and BTB domain containing 7B) | 51043 |
| ZEB1 (zinc finger E-box binding homeobox 1) | 6935 |
| ZEB2 (zinc finger E-box binding homeobox 2) | 9839 |
| ZFHX2 (zinc finger homeobox 2) | 85446 |
| ZFHX3 (zinc finger homeobox 3) | 463 |
| ZFHX4 (zinc finger homeobox 4) | 79776 |
| ZFP36L1 (zinc finger protein 36, C3H type-like 1) | 677 |
| ZFP36L2 (zinc finger protein 36, C3H type-like 2) | 678 |
| ZFP37 (zinc finger protein 37 homolog (mouse)) | 7539 |
| ZFP42 (zinc finger protein 42 homolog (mouse)) | 132625 |
| ZFPM2 (zinc finger protein, multitype 2) | 23414 |
| ZHX1 (zinc fingers and homeoboxes 1) | 11244 |
| ZHX2 (zinc fingers and homeoboxes 2) | 22882 |
| ZHX3 (zinc fingers and homeoboxes 3) | 23051 |
| ZIC1 (Zic family member 1 (odd-paired homolog, Drosophila)) | 7545 |
| ZKSCAN1 (zinc finger with KRAB and SCAN domains 1) | 7586 |
| ZKSCAN2 (zinc finger with KRAB and SCAN domains 2) | 342357 |
| ZKSCAN3 (zinc finger with KRAB and SCAN domains 3) | 80317 |
| ZKSCAN4 (zinc finger with KRAB and SCAN domains 4) | 387032 |
| ZKSCAN5 (zinc finger with KRAB and SCAN domains 5) | 23660 |
| ZNF117 (zinc finger protein 117) | 51351 |
| ZNF131 (zinc finger protein 131) | 7690 |
| ZNF132 (zinc finger protein 132) | 7691 |
| ZNF133 (zinc finger protein 133) | 7692 |
| ZNF134 (zinc finger protein 134) | 7693 |
| ZNF135 (zinc finger protein 135) | 7694 |
| ZNF136 (zinc finger protein 136) | 7695 |
| ZNF138 (zinc finger protein 138) | 7697 |
| ZNF140 (zinc finger protein 140) | 7699 |
| ZNF141 (zinc finger protein 141) | 7700 |
| ZNF142 (zinc finger protein 142) | 7701 |
| ZNF143 (zinc finger protein 143) | 7702 |
| ZNF148 (zinc finger protein 148) | 7707 |
| ZNF154 (zinc finger protein 154) | 7710 |
| ZNF155 (zinc finger protein 155) | 7711 |
| ZNF157 (zinc finger protein 157) | 7712 |
| ZNF165 (zinc finger protein 165) | 7718 |
| ZNF167 (zinc finger protein 167) | 55888 |
| ZNF169 (zinc finger protein 169) | 169841 |
| ZNF174 (zinc finger protein 174) | 7727 |
| ZNF175 (zinc finger protein 175) | 7728 |
| ZNF18 (zinc finger protein 18) | 7566 |
| ZNF187 (zinc finger protein 187) | 7741 |
| ZNF189 (zinc finger protein 189) | 7743 |
| ZNF19 (zinc finger protein 19) | 7567 |
| ZNF192 (zinc finger protein 192) | 7745 |
| ZNF193 (zinc finger protein 193) | 7746 |
| ZNF197 (zinc finger protein 197) | 10168 |
| ZNF202 (zinc finger protein 202) | 7753 |
| ZNF207 (zinc finger protein 207) | 7756 |
| ZNF211 (zinc finger protein 211) | 10520 |
| ZNF213 (zinc finger protein 213) | 7760 |
| ZNF215 (zinc finger protein 215) | 7762 |
| ZNF217 (zinc finger protein 217) | 7764 |
| ZNF219 (zinc finger protein 219) | 51222 |
| ZNF232 (zinc finger protein 232) | 7775 |
| ZNF236 (zinc finger protein 236) | 7776 |
| ZNF238 (zinc finger protein 238) | 10472 |
| ZNF24 (zinc finger protein 24) | 7572 |
| ZNF256 (zinc finger protein 256) | 10172 |
| ZNF263 (zinc finger protein 263) | 10127 |
| ZNF268 (zinc finger protein 268) | 10795 |
| ZNF274 (zinc finger protein 274) | 10782 |
| ZNF277 (zinc finger protein 277) | 11179 |
| ZNF281 (zinc finger protein 281) | 23528 |
| ZNF287 (zinc finger protein 287) | 57336 |
| ZNF3 (zinc finger protein 3) | 7551 |
| ZNF323 (zinc finger protein 323) | 64288 |
| ZNF33A (zinc finger protein 33A) | 7581 |
| ZNF33B (zinc finger protein 33B) | 7582 |
| ZNF345 (zinc finger protein 345) | 25850 |
| ZNF35 (zinc finger protein 35) | 7584 |
| ZNF354A (zinc finger protein 354A) | 6940 |
| ZNF367 (zinc finger protein 367) | 195828 |
| ZNF37A (zinc finger protein 37A) | 7587 |
| ZNF394 (zinc finger protein 394) | 84124 |
| ZNF396 (zinc finger protein 396) | 252884 |
| ZNF397 (zinc finger protein 397) | 84307 |
| ZNF397OS (zinc finger protein 397 opposite strand) | 100101467 |
| ZNF41 (zinc finger protein 41) | 7592 |
| ZNF423 (zinc finger protein 423) | 23090 |
| ZNF444 (zinc finger protein 444) | 55311 |
| ZNF445 (zinc finger protein 445) | 353274 |
| ZNF446 (zinc finger protein 446) | 55663 |
| ZNF449 (zinc finger protein 449) | 203523 |
| ZNF45 (zinc finger protein 45) | 7596 |
| ZNF483 (zinc finger protein 483) | 158399 |
| ZNF496 (zinc finger protein 496) | 84838 |
| ZNF498 (zinc finger protein 498) | 221785 |
| ZNF500 (zinc finger protein 500) | 26048 |
| ZNF628 (zinc finger protein 628) | 89887 |
| ZNF69 (zinc finger protein 69) | 7620 |
| ZNF70 (zinc finger protein 70) | 7621 |
| ZNF71 (zinc finger protein 71) | 58491 |
| ZNF73 (zinc finger protein 73) | 7624 |
| ZNF75C (zinc finger protein 75C pseudogene) | 7777 |
| ZNF75D (zinc finger protein 75D) | 7626 |
| ZNF80 (zinc finger protein 80) | 7634 |
| ZNF81 (zinc finger protein 81) | 347344 |
| ZNF83 (zinc finger protein 83) | 55769 |
| ZNF85 (zinc finger protein 85) | 7639 |
| ZNF90 (zinc finger protein 90) | 7643 |
| ZNF91 (zinc finger protein 91) | 7644 |
| ZNF92 (zinc finger protein 92) | 168374 |
| ZNF93 (zinc finger protein 93) | 81931 |
| ZNFX1 (zinc finger, NFX1-type containing 1) | 57169 |
| ZRANB2 (zinc finger, RAN-binding domain containing 2) | 9406 |
| ZSCAN1 (zinc finger and SCAN domain containing 1) | 284312 |
| ZSCAN10 (zinc finger and SCAN domain containing 10) | 84891 |
| ZSCAN12 (zinc finger and SCAN domain containing 12) | 9753 |
| ZSCAN16 (zinc finger and SCAN domain containing 16) | 80345 |
| ZSCAN18 (zinc finger and SCAN domain containing 18) | 65982 |
| ZSCAN2 (zinc finger and SCAN domain containing 2) | 54993 |
| ZSCAN20 (zinc finger and SCAN domain containing 20) | 7579 |
| ZSCAN21 (zinc finger and SCAN domain containing 21) | 7589 |
| ZSCAN22 (zinc finger and SCAN domain containing 22) | 342945 |
| ZSCAN23 (zinc finger and SCAN domain containing 23) | 222696 |
| ZSCAN29 (zinc finger and SCAN domain containing 29) | 146050 |
| ZSCAN4 (zinc finger and SCAN domain containing 4) | 201516 |
| ZSCAN5A (zinc finger and SCAN domain containing 5A) | 79149 |
| ZSCAN5B (zinc finger and SCAN domain containing 5B) | 342933 |
| ZSCAN5C (zinc finger and SCAN domain containing 5C) | 649137 |

5.9 Representative Compounds that May be Screened

In some embodiments, any combination of the compounds listed in Table 2 and/or Table 3 may be screened in step 202, described above. In some embodiments, any combination of the compounds listed in Table 2 and/or Table 3 may be screened in step 202 in addition to compounds not listed in this section. In some embodiments, compounds not listed Table 2 and/or Table 3 are screened in step 202.

Each of the 1040 compounds listed in Table 2 has reached clinical trial stages in the United States. Each of the compounds listed in Table 2 has been assigned USAN or USP status and is included in the USP Dictionary (U.S. Pharmacopeia, 2005), the authorized list of established names for drugs in the USA. These compounds are available, for screening purposes, from MicroSource Discovery Systems, Inc. (MDSI) (Gaylordsville, Conn.).

Table 3 is a collection of natural products comprising alkaloids (16%), flavanoids (12%), sterols/triterpenes (12%), diterpenes/sesquiterpenes (10%), enzophenones/chalcones/stilbenes (10%), limonoids/quassinoids (9%), and chromones/coumarins (6%). The remainder of the collection includes quinones/quinonemethides, benzofurans/benzopyrans, rotenoids/xanthones, carbohydrates, and benztropolones/depsides/depsidones, in descending order. These compounds are available, for screening purposes, from MDSI. See A Vogt, A Tamewitz, J Skoko, R P Sikorski, K A Giuliano and J S Lazo, “The Benzo[c]phenanthridine Alkaloid, Sanguinarine, Is a Selective, Cell-active Inhibitor of Mitogen-activated Protein Kinase Phosphatase-1”, J Biol Chem 280:19078 (2005), which is hereby incorporated by reference herein in its entirety.

TABLE 2
Exemplary Screening Compounds (From Clinical Trials)

| Compound Name | Compound Name | Compound Name |
|---|---|---|
| ACARBOSE | ACEBUTOLOL | ACECAINIDE |
| | HYDROCHLORIDE | HYDROCHLORIDE |
| ACECLIDINE | ACEDAPSONE | ACEPROMAZINE MALEATE |
| HYDROCHLORIDE | | |
| ACETAMINOPHEN | ACETAZOLAMIDE | ACETOHEXAMIDE |
| ACETOHYDROXAMIC ACID | ACETRIAZOIC ACID | ACETYLCHOLINE |
| ACETYLCYSTEINE | ACIVICIN | ACLACINOMYCIN A |
| ACRISORCIN | ACTINOQUINOL SODIUM | ACYCLOVIR |
| ADENINE | ADENOSINE | ADENOSINE PHOSPHATE |
| ADIPHENINE | ADRENALINE BITARTRATE | AKLOMIDE |
| HYDROCHLORIDE | | |
| ALAPROCLATE | ALBENDAZOLE | ALBUTEROL (+/−) |
| ALCLOMETAZONE | ALENDRONATE SODIUM | ALFLUZOCIN |
| DIPROPIONATE | | |
| ALLANTOIN | ALLOPURINOL | ALMOTRIPTAN |
| ALPHA-TOCHOPHEROL | ALPHA-TOCHOPHERYL | ALPRENOLOL |
| | ACETATE | |
| ALRESTATIN | ALTHIAZIDE | ALTRETAMINE |
| ALVERINE CITRATE | AMANTADINE | AMCINONIDE |
| | HYDROCHLORIDE | |
| AMFENAC | AMIDAPSONE | AMIFOSTINE |
| AMIKACIN SULFATE | AMILORIDE | AMINACRINE |
| | HYDROCHLORIDE | |
| AMINOCAPROIC ACID | AMINOGLUTETHIMIDE | AMINOHIPPURIC ACID |
| AMINOLEVULINIC ACID | AMINOPENTAMIDE | AMINOREX |
| HYDROCHLORIDE | | |
| AMINOSALICYLATE SODIUM | AMIODARONE | AMIPRILOSE |
| | HYDROCHLORIDE | |
| AMITRAZ | AMITRIPTYLINE | AMLODIPINE BESYLATE |
| | HYDROCHLORIDE | |
| AMODIAQUINE | AMOXAPINE | AMOXICILLIN |
| DIHYDROCHLORIDE | | |
| AMPHOTERICIN B | AMPICILLIN SODIUM | AMPROLIUM |
| AMSACRINE | ANAGRELIDE | ANIRACETAM |
| | HYDROCHLORIDE | |
| ANTAZOLINE PHOSPHATE | ANTHRALIN | ANTIPYRINE |
| APOMORPHINE | APRAMYCIN | ARILIDONE |
| HYDROCHLORIDE | | |
| ARPRINOCID | ARSANILIC ACID | ASCORBIC ACID |
| ASPARTAME | ASPIRIN | ASTEMIZOLE |
| ATENOLOL | ATOMOXETINE | ATORVASTATIN CALCIUM |
| | HYDROCHLORIDE | |
| ATOVAQUONE | ATROPINE OXIDE | ATROPINE SULFATE |
| AUROTHIOGLUCOSE | AVERMECTIN B1 | AVOBENZONE |
| AZACITIDINE | AZAPERONE | AZASERINE |
| AZATHIOPRINE | AZELAIC ACID | AZELASTINE |
| | | HYDROCHLORIDE |
| AZITHROMYCIN | AZLOCILLIN SODIUM | AZTREONAM |
| BACAMPICILLIN | BACITRACIN | BACLOFEN |
| HYDROCHLORIDE | | |
| BECLOMETHASONE | BEKANAMYCIN SULFATE | BELOXAMIDE |
| DIPROPIONATE | | |
| BENAZEPRIL | BENDAZAC | BENDROFLUMETHIAZIDE |
| HYDROCHLORIDE | | |
| BENSERAZIDE | BENURESTAT | BENZALKONIUM CHLORIDE |
| HYDROCHLORIDE | | |
| BENZETHONIUM CHLORIDE | BENZOCAINE | BENZOXYQUINE |
| BENZOYL PEROXIDE | BENZOYLPAS | BENZTHIAZIDE |
| BENZTROPINE | BENZYL BENZOATE | BEPRIDIL HYDROCHLORIDE |
| BETA-CAROTENE | BETA-PROPIOLACTONE | BETAHISTINE |
| | | HYDROCHLORIDE |
| BETAINE HYDROCHLORIDE | BETAMETHASONE | BETAMETHASONE 17,21- |
| | | DIPROPIONATE |
| BETAMETHASONE | BETHANECHOL CHLORIDE | BEZAFIBRATE |
| VALERATE | | |
| BIFONAZOLE | BIOTIN | BIPERIDEN |
| BISACODYL | BISMUTH SUBSALICYLATE | BITHIONATE SODIUM |
| BLEOMYCIN (BLEOMYCIN B2 | BRETYLIUM TOSYLATE | BROMHEXINE |
| SHOWN) | | HYDROCHLORIDE |
| BROMINDIONE | BROMOCRIPTINE MESYLATE | BROMPHENIRAMINE |
| | | MALEATE |
| BUDESONIDE | BUMETANIDE | BUNAMIDINE |
| | | HYDROCHLORIDE |
| BUPIVACAINE | BUPROPION | BURAMATE |
| HYDROCHLORIDE | | |
| BUSPIRONE | BUSULFAN | BUTACAINE |
| HYDROCHLORIDE | | |
| BUTACETIN | BUTAMBEN | BUTENAFINE |
| | | HYDROCHLORIDE |
| BUTIROSIN SULFATE | BUTOCONAZOLE | CAFFEINE |
| CAMPHOR (1R) | CANDESARTAN CILEXTIL | CANRENOIC ACID, |
| | | POTASSIUM SALT |
| CANRENONE | CAPOBENIC ACID | CAPREOMYCIN SULFATE |
| CAPSAICIN | CAPTOPRIL | CARBACHOL |
| CARBADOX | CARBAMAZEPINE | CARBENICILLIN DISODIUM |
| CARBENOXOLONE SODIUM | CARBIDOPA | CARBINOXAMINE MALEATE |
| CARBOPLATIN | CARISOPRODOL | CARMUSTINE |
| CARPROFEN | CARVEDILOL TARTRATE | CEFACLOR |
| CEFADROXIL | CEFAMANDOLE NAFATE | CEFAMANDOLE SODIUM |
| CEFAZOLIN SODIUM | CEFDINIR | CEFMETAZOLE SODIUM |
| CEFONICID SODIUM | CEFOPERAZONE SODIUM | CEFOTAXIME SODIUM |
| CEFOXITIN SODIUM | CEFPODOXIME PROXETIL | CEFPROZIL |
| CEFSULODIN SODIUM | CEFTIBUTEN | CEFTRIAXONE SODIUM |
| CEFTRIAXONE SODIUM | CEFUROXIME AXETIL | CEFUROXIME SODIUM |
| TRIHYDRATE | | |
| CELECOXIB | CEPHALEXIN | CEPHALOGLYCINE |
| CEPHALORIDINE | CEPHALOTHIN SODIUM | CEPHAPIRIN SODIUM |
| CEPHRADINE | CETABEN | CETIRIZINE |
| | | HYDROCHLORIDE |
| CETYLPYRIDINIUM | CHENODIOL | CHLORAMBUCIL |
| CHLORIDE | | |
| CHLORAMPHENICOL | CHLORAMPHENICOL | CHLORAMPHENICOL |
| | HEMISUCCINATE | PALMITATE |
| CHLORAMPHENICOL | CHLORCYCLIZINE | CHLORHEXIDINE |
| PALMITATE | HYDROCHLORIDE | |
| CHLORMADINONE ACETATE | CHLOROGUANIDE | CHLOROPHYLLIDE CU |
| | HYDROCHLORIDE | COMPLEX NA SALT |
| CHLOROQUINE | CHLOROTHIAZIDE | CHLOROTRIANISENE |
| DIPHOSPHATE | | |
| CHLOROXINE | CHLOROXYLENOL | CHLORPHENIRAMINE (S) |
| | | MALEATE |
| CHLORPROMAZINE | CHLORPROPAMIDE | CHLORPROTHIXENE |
| | | HYDROCHLORIDE |
| CHLORTETRACYCLINE | CHLORTHALIDONE | CHLORZOXAZONE |
| HYDROCHLORIDE | | |
| CHOLECALCIFEROL | CHOLINE CHLORIDE | CICLOPIROX OLAMINE |
| CILOSTAZOL | CIMETIDINE | CINNARAZINE |
| CINOXACIN | CIPROFIBRATE | CIPROFLOXACIN |
| CISPLATIN | CITALOPRAM | CITICOLINE |
| CLARITHROMYCIN | CLAVULANATE LITHIUM | CLEBOPRIDE MALEATE |
| CLEMASTINE | CLIDINIUM BROMIDE | CLINDAMYCIN |
| | | HYDROCHLORIDE |
| CLINDAMYCIN PALMITATE | CLIOQUINOL | CLOBETASOL PROPIONATE |
| HYDROCHLORIDE | | |
| CLOFIBRATE | CLOMIPHENE CITRATE | CLOMIPRAMINE |
| | | HYDROCHLORIDE |
| CLONIDINE | CLOPAMIDE | CLOPIDOGREL SULFATE |
| HYDROCHLORIDE | | |
| CLOPIDOL | CLOPROSTENOL SODIUM | CLORSULON |
| CLOTRIMAZOLE | CLOXACILLIN SODIUM | CLOXYQUIN |
| CLOZAPINE | COENZYME B12 | COLCHICINE |
| COLFORSIN | COLISTIMETHATE SODIUM | CORTISONE ACETATE |
| CROMOLYN SODIUM | CROTAMITON | CYCLIZINE |
| CYCLOBENZAPRINE | CYCLOHEXIMIDE | CYCLOPENTOLATE |
| HYDROCHLORIDE | | HYDROCHLORIDE |
| CYCLOPHOSPHAMIDE | CYCLOSERINE | CYCLOSPORINE |
| HYDRATE | | |
| CYCLOTHIAZIDE | CYPROTERONE ACETATE | CYTARABINE |
| D-LACTITOL MONOHYDRATE | DACARBAZINE | DACTINOMYCIN |
| DANAZOL | DANTROLENE SODIUM | DAPSONE |
| DARIFENACIN | DAUNORUBICIN | DEFEROXAMINE MESYLATE |
| DEHYDROCHOLIC ACID | DEMECLOCYCLINE | DERACOXIB |
| | HYDROCHLORIDE | |
| DESIPRAMINE | DESLORATIDINE | DESOXYCORTICOSTERONE |
| HYDROCHLORIDE | | ACETATE |
| DESOXYCORTICOSTERONE | DEXAMETHASONE | DEXAMETHASONE ACETATE |
| PIVALATE | | |
| DEXAMETHASONE SODIUM | DEXCHLORPHENIRAMINE | DEXPANTHENOL |
| PHOSPHATE | MALEATE | |
| DEXPROPRANOLOL | DEXTROMETHORPHAN | DIAZEPAM |
| HYDROCHLORIDE | HYDROBROMIDE | |
| DIAZIQUONE | DIAZOXIDE | DIBENZOTHIOPHENE |
| DIBUCAINE | DICHLORPHENAMIDE | DICHLORVOS |
| HYDROCHLORIDE | | |
| DICLOFENAC SODIUM | DICLOXACILLIN SODIUM | DICUMAROL |
| DICYCLOMINE | DIENESTROL | DIETHYLCARBAMAZINE |
| HYDROCHLORIDE | | CITRATE |
| DIETHYLSTILBESTROL | DIETHYLTOLUAMIDE | DIFLUCORTOLONE PIVALATE |
| DIFLUNISAL | DIGITOXIN | DIGOXIN |
| DIHYDROERGOTAMINE | DIHYDROSTREPTOMYCIN | DILOXANIDE FUROATE |
| MESYLATE | SULFATE | |
| DILTIAZEM | DIMENHYDRINATE | DIMERCAPROL |
| HYDROCHLORIDE | | |
| DIMETHADIONE | DIOXYBENZONE | DIPHEMANIL METHYL |
| | | SULFATE |
| DIPHENHYDRAMINE | DIPHENYLPYRALINE | DIPYRIDAMOLE |
| HYDROCHLORIDE | HYDROCHLORIDE | |
| DIPYRONE | DIRITHROMYCIN | DISOPYRAMIDE PHOSPHATE |
| DISULFIRAM | DOBUTAMINE | DOMPERIDONE |
| | HYDROCHLORIDE | |
| DONEPEZIL | DOPAMINE | DOXEPIN HYDROCHLORIDE |
| HYDROCHLORIDE | HYDROCHLORIDE | |
| DOXORUBICIN | DOXYCYCLINE | DOXYLAMINE SUCCINATE |
| | HYDROCHLORIDE | |
| DROPERIDOL | DULOXETINE | DUTASTERIDE |
| | HYDROCHLORIDE | |
| DYCLONINE | DYDROGESTERONE | DYPHYLLINE |
| HYDROCHLORIDE | | |
| ECONAZOLE NITRATE | EDOXUDINE | EDROPHONIUM CHLORIDE |
| ELETRIPTAN | EMETINE | ENALAPRIL MALEATE |
| HYDROBROMIDE | | |
| ENOXACIN | ENOXAPARIN SODIUM (1% WT/ | ENROFLOXACIN |
| | VOL IN 10% AQ DMSO) | |
| EPHEDRINE (1R,2S) | EQUILIN | ERGOCALCIFEROL |
| HYDROCHLORIDE | | |
| ERGONOVINE MALEATE | ERYTHROMYCIN | ERYTHROMYCIN |
| | | ETHYLSUCCINATE |
| ESCITALOPRAM OXALATE | ESTRADIOL | ESTRADIOL ACETATE |
| ESTRADIOL CYPIONATE | ESTRADIOL VALERATE | ESTRIOL |
| ESTRONE | ESTROPIPATE | ESZOPICLONE |
| ETANIDAZOLE | ETHACRYNIC ACID | ETHAMBUTOL |
| | | HYDROCHLORIDE |
| ETHINYL ESTRADIOL | ETHIONAMIDE | ETHOPABATE |
| ETHOPROPAZINE | ETHOSUXIMIDE | ETHOTOIN |
| HYDROCHLORIDE | | |
| ETHOXZOLAMIDE | ETHYLNOREPINEPHRINE | ETIDRONATE DISODIUM |
| | HYDROCHLORIDE | |
| ETODOLAC | ETOMIDATE | ETOPOSIDE |
| EUCATROPINE | EUGENOL | EXEMESTANE |
| HYDROCHLORIDE | | |
| EZETIMIBE | FAMCICLOVIR | FAMOTIDINE |
| FAMPRIDINE | FENBENDAZOLE | FENBUFEN |
| FENOPROFEN | FENOTEROL | FENSPIRIDE |
| | HYDROBROMIDE | HYDROCHLORIDE |
| FEXOFENADINE | FILIPIN | FLOXURIDINE |
| HYDROCHLORIDE | | |
| FLUCONAZOLE | FLUCYTOSINE | FLUDROCORTISONE |
| | | ACETATE |
| FLUFENAMIC ACID | FLUMEQUINE | FLUMETHASONE |
| FLUMETHAZONE PIVALATE | FLUNARIZINE | FLUNISOLIDE |
| | HYDROCHLORIDE | |
| FLUNIXIN | FLUNIXIN MEGLUMINE | FLUOCINOLONE ACETONIDE |
| FLUOCINONIDE | FLUORESCEIN | FLUOROMETHOLONE |
| FLUOROURACIL | FLUOXETINE | FLUPHENAZINE |
| | | HYDROCHLORIDE |
| FLURANDRENOLIDE | FLURBIPROFEN | FLUROTHYL |
| FLUTAMIDE | FOLIC ACID | FOMEPIZOLE |
| | | HYDROCHLORIDE |
| FOSCARNET SODIUM | FOSFOMYCIN | FOSINOPRIL SODIUM |
| FROVATRIPTAN | FURAZOLIDONE | FUREGRELATE SODIUM |
| FUROSEMIDE | FUSIDIC ACID | GABAPENTIN |
| GALANTHAMINE | GALLAMINE TRIETHIODIDE | GATIFLOXACIN |
| HYDROBROMIDE | | |
| GEMFIBROZIL | GEMIFLOXACIN MESYLATE | GENTAMICIN SULFATE |
| GENTIAN VIOLET | GLIPIZIDE | GLUCONOLACTONE |
| GLUCOSAMINE | GLYBURIDE | GRAMICIDIN |
| HYDROCHLORIDE | | |
| GRISEOFULVIN | GUAIFENESIN | GUANABENZ ACETATE |
| GUANETHIDINE SULFATE | GUANFACINE | HALAZONE |
| | HYDROCHLORIDE | |
| HALCINONIDE | HALOPERIDOL | HALOPROGIN |
| HALOTHANE | HETACILLIN POTASSIUM | HEXACHLOROPHENE |
| HEXYLRESORCINOL | HISTAMINE | HOMATROPINE BROMIDE |
| | DIHYDROCHLORIDE | |
| HOMATROPINE | HOMOSALATE | HYCANTHONE |
| METHYLBROMIDE | | |
| HYDRALAZINE | HYDRASTINE (1S,9R) | HYDROCHLOROTHIAZIDE |
| HYDROCHLORIDE | | |
| HYDROCORTISONE | HYDROCORTISONE ACETATE | HYDROCORTISONE |
| | | BUTYRATE |
| HYDROCORTISONE | HYDROCORTISONE SODIUM | HYDROCORTISONE |
| HEMISUCCINATE | PHOSPHATE | VALERATE |
| HYDROFLUMETHIAZIDE | HYDROQUINONE | HYDROXYAMPHETAMINE |
| | | HYDROBROMIDE |
| HYDROXYCHLOROQUINE | HYDROXYPROGESTERONE | HYDROXYUREA |
| SULFATE | CAPROATE | |
| HYDROXYZINE PAMOATE | HYOSCYAMINE | IBUPROFEN |
| IFOSFAMIDE | IMIPRAMINE | INDAPAMIDE |
| | HYDROCHLORIDE | |
| INDOMETHACIN | INDOPROFEN | INDORAMIN |
| | | HYDROCHLORIDE |
| IODIPAMIDE | IODOQUINOL | IOPANIC ACID |
| IPRATROPIUM BROMIDE | IRBESARTAN | IRINOTECAN |
| | | HYDROCHLORIDE |
| ISONIAZID | ISOPROPAMIDE IODIDE | ISOPROTERENOL |
| | | HYDROCHLORIDE |
| ISOSORBIDE DINITRATE | ISOTRETINON | ISOXICAM |
| ISOXSUPRINE | KANAMYCIN A SULFATE | KETANSERIN TARTRATE |
| HYDROCHLORIDE | | |
| KETOCONAZOLE | KETOPROFEN | KETOROLAC |
| | | TROMETHAMINE |
| KETOTIFEN FUMARATE | LABETALOL | LACTULOSE |
| | HYDROCHLORIDE | |
| LAMOTRIGINE | LANSOPRAZOLE | LASALOCID SODIUM |
| LEFUNOMIDE | LEUCOVORIN CALCIUM | LEVALBUTEROL |
| | | HYDROCHLORIDE |
| LEVAMISOLE | LEVOBUNOLOL | LEVOCARNITINE |
| HYDROCHLORIDE | HYDROCHLORIDE | |
| LEVODOPA | LEVOFLOXACIN | LEVONORDEFRIN |
| LIDOCAINE | LIFIBRATE | LINCOMYCIN |
| HYDROCHLORIDE | | HYDROCHLORIDE |
| LINDANE | LIOTHYRONINE | LIOTHYRONINE (L-ISOMER) |
| | | SODIUM |
| LISINOPRIL | LITHIUM CITRATE | LOBENDAZOLE |
| LOBENZARIT | LOMEFLOXACIN | LOPERAMIDE |
| | HYDROCHLORIDE | HYDROCHLORIDE |
| LORATADINE | LOSARTAN | LOVASTATIN |
| LOXAPINE SUCCINATE | LUPITIDINE | MAFENIDE HYDROCHLORIDE |
| | HYDROCHLORIDE | |
| MALATHION | MAPROTILINE | MEBENDAZOLE |
| | HYDROCHLORIDE | |
| MEBEVERINE | MECAMYLAMINE | MECHLORETHAMINE |
| HYDROCHLORIDE | HYDROCHLORIDE | |
| MECLIZINE | MECLOCYCLINE | MECLOFENAMATE SODIUM |
| HYDROCHLORIDE | SULFOSALICYLATE | |
| MEDROXYPROGESTERONE | MEDRYSONE | MEFENAMIC ACID |
| ACETATE | | |
| MEFEXAMIDE | MEFLOQUINE | MEGESTROL ACETATE |
| MELOXICAM | MELPHALAN | MEMANTINE |
| | | HYDROCHLORIDE |
| MENADIONE | MENTHOL(−) | MEPARTRICIN |
| MEPENZOLATE BROMIDE | MEPHENTERMINE SULFATE | MEPHENYTOIN |
| MEPIVACAINE | MERCAPTOPURINE | MESNA |
| HYDROCHLORIDE | | |
| MESTRANOL | METAPROTERENOL | METARAMINOL BITARTRATE |
| METAXALONE | METFORMIN | METHACHOLINE CHLORIDE |
| | HYDROCHLORIDE | |
| METHACYCLINE | METHAPYRILENE | METHAZOLAMIDE |
| HYDROCHLORIDE | HYDROCHLORIDE | |
| METHENAMINE | METHICILLIN SODIUM | METHIMAZOLE |
| METHOCARBAMOL | METHOTREXATE(+/−) | METHOXAMINE |
| | | HYDROCHLORIDE |
| METHOXSALEN | METHSCOPOLAMINE | METHSUXIMIDE |
| | BROMIDE | |
| METHYLATROPINE NITRATE | METHYLBENZETHONIUM | METHYLDOPA |
| | CHLORIDE | |
| METHYLDOPATE | METHYLENE BLUE | METHYLERGONOVINE |
| HYDROCHLORIDE | | MALEATE |
| METHYLPREDNISOLONE | METHYLPREDNISOLONE | METHYLTHIOURACIL |
| | SODIUM SUCCINATE | |
| METOCLOPRAMIDE | METOLAZONE | METOPROLOL TARTRATE |
| HYDROCHLORIDE | | |
| METRONIDAZOLE | MEXILETINE | MIANSERIN |
| | HYDROCHLORIDE | HYDROCHLORIDE |
| MICONAZOLE NITRATE | MIDODRINE | MIFEPRISTONE |
| | HYDROCHLORIDE | |
| MIGLITOL | MINAPRINE | MINOCYCLINE |
| | HYDROCHLORIDE | HYDROCHLORIDE |
| MINOXIDIL | MITOMYCIN C | MITOTANE |
| MITOXANTHRONE | MODAFINIL | MOEXIPRIL |
| HYDROCHLORIDE | | HYDROCHLORIDE |
| MOLSIDOMINE | MOMETASONE FUROATE | MONENSIN SODIUM |
| | | (MONENSIN A IS SHOWN) |
| MONOBENZONE | MONTELUKAST SODIUM | MORANTEL CITRATE |
| MOXALACTAM DISODIUM | MOXIDECTIN | MOXIFLOXACIN |
| | | HYDROCHLORIDE |
| MYCOPHENOLIC ACID | NABUMETONE | NADIDE |
| NADOLOL | NAFCILLIN SODIUM | NAFOXIDINE |
| | | HYDROCHLORIDE |
| NAFRONYL OXALATE | NAFTIFINE HYDROCHLORIDE | NALBUPHINE |
| | | HYDROCHLORIDE |
| NALIDIXIC ACID | NALOXONE | NALTREXONE |
| | HYDROCHLORIDE | HYDROCHLORIDE |
| NAPHAZOLINE | NAPROXEN(+) | NAPROXOL |
| HYDROCHLORIDE | | |
| NARASIN | NATAMYCIN | NATEGLINIDE |
| NEFOPAM | NEOMYCIN SULFATE | NEOSTIGMINE BROMIDE |
| NETILMICIN | NIACIN | NIACINAMIDE |
| NICARDIPINE | NICERGOLINE | NICLOSAMIDE |
| HYDROCHLORIDE | | |
| NICOTINE DITARTRATE | NICOTINYL ALCOHOL | NIFEDIPINE |
| | TARTRATE | |
| NIFURALDEZONE | NIFURPIRINOL | NILUTAMIDE |
| NIMODIPINE | NISOLDIPINE | NITHIAMIDE |
| NITRENDIPINE | NITROFURANTOIN | NITROFURAZONE |
| NITROMERSOL | NITROMIDE | NOCODAZOLE |
| NOMIFENSINE MALEATE | NONOXYNOL-9 | NOREPINEPHRINE |
| NORETHINDRONE | NORETHINDRONE ACETATE | NORETHYNODREL |
| NORFLOXACIN | NORGESTIMATE | NORGESTREL |
| NORTRIPTYLINE | NOSCAPINE | NOVOBIOCIN SODIUM |
| | HYDROCHLORIDE | |
| NYLIDRIN HYDROCHLORIDE | NYSTATIN | OCTODRINE |
| OFLOXACIN | OLANZAPINE | OLMESARTAN |
| OLMESARTAN MEDOXOMIL | OLVANIL | OMEPRAZOLE |
| ORLISTAT | ORPHENADRINE CITRATE | OSELTAMIVIR TARTRATE |
| OUABAIN | OXACILLIN SODIUM | OXAPROZIN |
| OXCARBAZEPINE | OXETHAZAINE | OXFENDAZOLE |
| OXIBENDAZOLE | OXICONAZOLE NITRATE | OXIDOPAMINE |
| | | HYDROCHLORIDE |
| OXOLINIC ACID | OXYBENZONE | OXYBUTYNIN CHLORIDE |
| OXYMETAZOLINE | OXYPHENBUTAZONE | OXYPHENCYCLIMINE |
| HYDROCHLORIDE | | HYDROCHLORIDE |
| OXYQUINOLINE | OXYTETRACYCLINE | PACLITAXEL |
| HEMISULFATE | | |
| PANCURONIUM BROMIDE | PANTHENOL | PANTOPRAZOLE |
| PANTOTHENIC ACID(D) CA | PAPAVERINE | PARACHLOROPHENOL |
| SALT | HYDROCHLORIDE | |
| PARAMETHADIONE | PARAROSANILINE PAMOATE | PARGYLINE |
| | | HYDROCHLORIDE |
| PAROMOMYCIN SULFATE | PAROXETINE | PEFLOXACINE MESYLATE |
| | HYDROCHLORIDE | |
| PEMOLINE | PENFLURIDOL | PENICILLAMINE |
| PENICILLIN G POTASSIUM | PENICILLIN V POTASSIUM | PENTOBARBITAL |
| PENTOXIFYLLINE | PERGOLIDE MESYLATE | PERHEXILINE MALEATE |
| PERINDOPRIL ERBUMINE | PERPHENAZINE | PHENACEMIDE |
| PHENACETIN | PHENAZOPYRIDINE | PHENELZINE SULFATE |
| | HYDROCHLORIDE | |
| PHENETHICILLIN | PHENINDIONE | PHENIRAMINE MALEATE |
| POTASSIUM | | |
| PHENOBARBITAL | PHENOLPHTHALEIN | PHENOXYBENZAMINE |
| | | HYDROCHLORIDE |
| PHENSUCCIMIDE | PHENTOLAMINE | PHENYLBUTAZONE |
| | HYDROCHLORIDE | |
| PHENYLEPHRINE | PHENYLETHYL ALCOHOL | PHENYTOIN SODIUM |
| HYDROCHLORIDE | | |
| PHTHALYLSULFATHIAZOLE | PHYSOSTIGMINE | PHYTONADIONE |
| | SALICYLATE | |
| PILOCARPINE NITRATE | PIMECROLIMUS | PIMOZIDE |
| PINACIDIL | PINDOLOL | PIOGLITAZONE |
| | | HYDROCHLORIDE |
| PIPAMPERONE | PIPERACILLIN SODIUM | PIPERAZINE |
| PIPERIDOLATE | PIPERINE | PIPOBROMAN |
| HYDROCHLORIDE | | |
| PIRACETAM | PIRENPERONE | PIRENZEPINE |
| | | HYDROCHLORIDE |
| PIRFENIDONE | PIROXICAM | PIZOTYLINE MALATE |
| PODOFILOX | POLYMYXIN B SULFATE | PONALRESTAT |
| POTASSIUM P- | PRACTOLOL | PRALIDOXIME CHLORIDE |
| AMINOBENZOATE | | |
| PRAMIPEXOLE | PRAMOXINE | PRAVASTATIN SODIUM |
| DIHYDROCHLORIDE | HYDROCHLORIDE | |
| PRAZIQUANTEL | PRAZOSIN HYDROCHLORIDE | PREDNISOLONE |
| PREDNISOLONE ACETATE | PREDNISOLONE | PREDNISOLONE TEBUTATE |
| | HEMISUCCINATE | |
| PREDNISONE | PREGABALIN | PRIFELONE |
| PRILOCAINE | PRIMAQUINE DIPHOSPHATE | PRIMIDONE |
| HYDROCHLORIDE | | |
| PROADIFEN | PROBENECID | PROBUCOL |
| HYDROCHLORIDE | | |
| PROCAINAMIDE | PROCAINE HYDROCHLORIDE | PROCATEROL |
| HYDROCHLORIDE | | HYDROCHLORIDE |
| PROCHLORPERAZINE | PROCYCLIDINE | PROGESTERONE |
| EDISYLATE | HYDROCHLORIDE | |
| PROGLUMIDE | PROMAZINE | PROMETHAZINE |
| | HYDROCHLORIDE | HYDROCHLORIDE |
| PROPAFENONE | PROPANTHELINE BROMIDE | PROPARACAINE |
| HYDROCHLORIDE | | HYDROCHLORIDE |
| PROPIOMAZINE MALEATE | PROPOFOL | PROPRANOLOL |
| | | HYDROCHLORIDE (+/−) |
| PROPYLTHIOURACIL | PSEUDOEPHEDRINE | PUROMYCIN |
| | HYDROCHLORIDE | HYDROCHLORIDE |
| PYRANTEL PAMOATE | PYRAZINAMIDE | PYRETHRINS |
| PYRIDOSTIGMINE BROMIDE | PYRIDOXINE | PYRILAMINE MALEATE |
| PYRIMETHAMINE | PYRITHIONE ZINC | PYRROCAINE |
| PYRVINIUM PAMOATE | QUETIAPINE | QUINACRINE |
| | | HYDROCHLORIDE |
| QUINAPRIL | QUINAPRILAT | QUINELORANE |
| HYDROCHLORIDE | | HYDROCHLORIDE |
| QUINIDINE GLUCONATE | QUININE SULFATE | QUINPIROLE |
| | | HYDROCHLORIDE |
| QUIPAZINE MALEATE | RACEPHEDRINE | RALOXIFENE |
| | HYDROCHLORIDE | HYDROCHLORIDE |
| RAMELTEON | RAMIPRIL | RANITIDINE |
| RANOLAZINE | RESERPINE | RESORCINOL |
| RESORCINOL | RIBAVIRIN | RIBOFLAVIN |
| MONOACETATE | | |
| RIFAMPIN | RIFAXIMIN | RILUZOLE |
| RIMANTADINE | RITANSERIN | RITODRINE |
| HYDROCHLORIDE | | HYDROCHLORIDE |
| RIVASTIGMINE | RIZATRIPTAN | ROBENIDINE |
| | | HYDROCHLORIDE |
| ROLIPRAM | ROLITETRACYCLINE | RONIDAZOLE |
| RONNEL | ROPINIROLE | ROSIGLITAZONE |
| ROSUVASTATIN | ROXARSONE | ROXITHROMYCIN |
| SALICIN | SALICYL ALCOHOL | SALICYLAMIDE |
| SALSALATE | SANGUINARINE SULFATE | SARAFLOXACIN |
| | | HYDROCHLORIDE |
| SCOPOLAMINE | SELAMECTIN | SEMUSTINE |
| HYDROBROMIDE | | |
| SENNOSIDE A | SERTRALINE | SIBUTRAMINE |
| | HYDROCHLORIDE | HYDROCHLORIDE |
| SILDENAFIL | SIMVASTATIN | SIROLIMUS |
| SISOMICIN SULFATE | SITAGLIPTIN | SODIUM DEHYDROCHOLATE |
| SODIUM SALICYLATE | SPARTEINE SULFATE | SPECTINOMYCIN |
| | | HYDROCHLORIDE |
| SPIPERONE | SPIRAMYCIN | SPIRONOLACTONE |
| STREPTOMYCIN SULFATE | STREPTOZOSIN | SUCCINYLSULFATHIAZOLE |
| SULCONAZOLE NITRATE | SULFABENZAMIDE | SULFACETAMIDE |
| SULFACHLORPYRIDAZINE | SULFADIAZINE | SULFADIMETHOXINE |
| SULFAMERAZINE | SULFAMETER | SULFAMETHAZINE |
| SULFAMETHIZOLE | SULFAMETHOXAZOLE | SULFAMETHOXYPYRIDAZINE |
| SULFAMONOMETHOXINE | SULFANILATE ZINC | SULFANITRAN |
| SULFAPYRIDINE | SULFAQUINOXALINE | SULFASALAZINE |
| | SODIUM | |
| SULFATHIAZOLE | SULFINPYRAZONE | SULFISOXAZOLE |
| SULFISOXAZOLE ACETYL | SULINDAC | SULISOBENZONE |
| SULOCTIDIL | SULPIRIDE | SUMATRIPTAN |
| SUPROFEN | SURAMIN | TACRINE HYDROCHLORIDE |
| TACROLIMUS | TADALAFIL | TAMOXIFEN CITRATE |
| TAMSULOSIN | TANNIC ACID | TAURINE |
| HYDRCHLORIDE | | |
| TEGASEROD MALEATE | TELITHROMYCIN | TELMISARTAN |
| TEMAZEPAM | TEMEFOS | TENIPOSIDE |
| TENOXICAM | TERAZOSIN | TERBINAFINE |
| | HYDROCHLORIDE | HYDROCHLORIDE |
| TERBUTALINE | TERFENADINE | TESTOSTERONE |
| HEMISULFATE | | |
| TESTOSTERONE | TETRACAINE | TETRACYCLINE |
| PROPIONATE | HYDROCHLORIDE | HYDROCHLORIDE |
| TETRAHYDROZOLINE | TETRAMIZOLE | TETROQUINONE |
| HYDROCHLORIDE | HYDROCHLORIDE | |
| THALIDOMIDE | THEOPHYLLINE | THIABENDAZOLE |
| THIAMINE | THIAMPHENICOL | THIAMYLAL SODIUM |
| THIMEROSAL | THIOGUANINE | THIOPENTAL SODIUM |
| THIORIDAZINE | THIOSTREPTON | THIOTEPA |
| HYDROCHLORIDE | | |
| THIOTHIXENE | THIRAM | THONZYLAMINE |
| | | HYDROCHLORIDE |
| TIAPRIDE HYDROCHLORIDE | TICARCILLIN DISODIUM | TICLOPIDINE |
| | | HYDROCHLORIDE |
| TILMICOSIN | TILORONE | TIMOLOL MALEATE |
| TINIDAZOLE | TIOCONAZOLE | TOBRAMYCIN |
| TOLAZAMIDE | TOLAZOLINE | TOLBUTAMIDE |
| | HYDROCHLORIDE | |
| TOLMETIN SODIUM | TOLNAFTATE | TOLTERODINE |
| | | HYDROCHLORIDE |
| TOLTRAZURIL | TOMELUKAST | TOPIRAMATE |
| TOPOTECAN | TOREMIPHENE CITRATE | TORSEMIDE |
| HYDROCHLORIDE | | |
| TRAMADOL | TRANEXAMIC ACID | TRANILAST |
| HYDROCHLORIDE | | |
| TRANYLCYPROMINE | TRAZODONE | TRETINON |
| SULFATE | HYDROCHLORIDE | |
| TRIACETIN | TRIAMCINOLONE | TRIAMCINOLONE |
| | | ACETONIDE |
| TRIAMCINOLONE | TRIAMTERENE | TRICHLORMETHIAZIDE |
| DIACETATE | | |
| TRICLOSAN | TRIENTINE | TRIFLUOPERAZINE |
| | | HYDROCHLORIDE |
| TRIFLUPERIDOL | TRIFLUPROMAZINE | TRIFLURIDINE |
| | HYDROCHLORIDE | |
| TRIHEXYPHENIDYL | TRIMEPRAZINE TARTRATE | TRIMETHADIONE |
| HYDROCHLORIDE | | |
| TRIMETHOBENZAMIDE | TRIMETHOPRIM | TRIMETOZINE |
| HYDROCHLORIDE | | |
| TRIMIPRAMINE MALEATE | TRIOXSALEN | TRIPELENNAMINE CITRATE |
| TRIPROLIDINE | TRISODIUM | TROGLITAZONE |
| HYDROCHLORIDE | ETHYLENEDIAMINE | |
| | TETRACETATE | |
| TROLEANDOMYCIN | TROPICAMIDE | TRYPTOPHAN |
| TUAMINOHEPTANE SULFATE | TUBOCURARINE CHLORIDE | TYLOSIN TARTRATE |
| TYROTHRICIN | UNDECYLENIC ACID | UREA |
| URSODIOL | VALACYCLOVIR | VALDECOXIB |
| | HYDROCHLORIDE | |
| VALPROATE SODIUM | VALSARTAN | VANCOMYCIN |
| | | HYDROCHLORIDE |
| VARDENAFIL | VARENICLINE | VENLAFAXINE |
| HYDROCHLORIDE | | |
| VERAPAMIL | VESAMICOL | VIDARABINE |
| HYDROCHLORIDE | HYDROCHLORIDE | |
| VIGABATRIN | VILOXAZINE | VINBLASTINE SULFATE |
| | HYDROCHLORIDE | |
| VINCRISTINE SULFATE | VINPOCETINE | WARFARIN |
| XYLAZINE | XYLOMETAZOLINE | YOHIMBINE |
| | HYDROCHLORIDE | HYDROCHLORIDE |
| ZIDOVUDINE [AZT] | ZIMELDINE | ZOLMITRIPTAN |
| | HYDROCHLORIDE | |
| ZOLPIDEM | ZOMEPIRAC SODIUM | |

TABLE 3
Natural Products for Screening

| Compound | Compound |
|---|---|
| 1(2)alpha-epoxydeoxydihydrogedunin | 1,2alpha-epoxy-7-deacetoxy-7-oxodihydrogedunin |
| 1,2alpha-epoxydeacetoxydihydrogedunin | 1,3-dideacetyl-7-deacetoxy-7-oxokhivorin |
| 1,3-dideacetyldeoxykhivorin | 1,4,5,8-tetrahydroxy-2,6-dimethylanthroquinone |
| 1,7-dideacetoxy-1,7-dioxo-3-deacetylkhivorin | 1,7-dideacetoxy-1,7-dioxokhivorin |
| 10-hydroxycamptothecin | 11alpha-acetoxykhivorin |
| 11-oxoursolic acid acetate | 12a-hydroxy-5-deoxydehydromunduserone |
| 12a-hydroxy-9-demethylmunduserone-8-carboxylic | 15-norcaryophyllen-3-one |
| acid | |
| 18alpha-glycyrrhetinic acid | 18-aminoabieta-8,11,13-triene sulfate |
| 19-hydroxytotarol | 1-methylxanthine |
| 1r,9s-hydrastine | 2,3-dihydroisogedunin |
| 2′,3-dihydroxy-4,4′,6′-trimethoxychalcone | 2,3-dihydroxy-4-methoxy-4′-ethoxybenzophenone |
| 2,3-methano-7,2′-dimethoxyflavanone | 2′,4-dihydroxy-3,4′,6′-trimethoxychalcone |
| 2′,4′-dihydroxy-3,4-dimethoxychalcone | 2′,4′-dihydroxy-4-methoxychalcone |
| 2′,4′-dihydroxychalcone 4′-glucoside | 2,5-dihydroxy-3,4-dimethoxy-4′- |
| | ethoxybenzophenone |
| 2,6-dimethoxyquinone | 2′,beta-dihydroxychalcone |
| 2-acetylpyrrole | 2-hydroxy-3,4-dimethoxybenzoic acid |
| 2-hydroxy-5 (6)epoxy-tetrahydrocaryophyllene | 2-hydroxyxanthone |
| 2-methoxy-5 (6)epoxy-tetrahydrocaryophyllene | 2′-methoxyformonetin |
| 2-methoxyresorcinol | 2-methoxyxanthone |
| 2-methyl gramine | 2-methylene-5-(2,5-dioxotetrahydrofuran-3-yl)-6- |
| | oxo--10,10-dimethylbicyclo[7:2:0]undecane |
| 2-propyl-3-hydroxyethylenepyran-4-one | 3,16-dideoxymexicanolide-3beta-diol |
| 3,4′,5,6,7-pentamethoxyflavone | 3,4,5-trimethoxycinnamaldehyde |
| 3′,4′-dihydroxyflavone | 3,4′-dihydroxyflavone |
| 3,4-dimethoxycinnamic acid | 3,4-dimethoxydalbergione |
| 3′,4′-dimethoxyflavone | 3,5-dihydroxyflavone |
| 3′,6-dihydroxyflavone | 3,7-dihydroxyflavone |
| 3,7-dimethoxyflavone | 3,7-epoxycaryophyllan-6-ol |
| 3,7-epoxycaryophyllan-6-one | 3alpha-acetoxydihydrodeoxygedunin |
| 3alpha-hydroxy-3-deoxyangolensic acid methyl ester | 3-alpha-hydroxydeoxygedinin |
| 3-amino-beta-pinene | 3beta,7beta- |
| | diacetoxydeoxodeacetoxydeoxydihydrogedunin |
| 3beta-acetoxydeoxodihydrogedunin | 3beta-acetoxydeoxyangolensic acid, methyl ester |
| 3beta-hydroxydeoxodihydrodeoxygedunin | 3beta-hydroxydeoxydesacetoxy-7-oxogedunin |
| 3-deacetylkhivorin | 3-deoxo-3beta-acetoxydeoxydihydrogedunin |
| 3-deoxo-3beta-hydroxymexicanolide 16-enol ether | 3-deoxy-3beta-hydroxyangolensic acid methyl ester |
| 3-deshydroxysappanol trimethyl ether | 3-hydroxy-4-(succin-2-yl)-caryolane delta-lactone |
| 3-hydroxycoumarin | 3-hydroxyflavone |
| 3-methoxycatechol | 3-methylorsellinic acid |
| 3-nor-3-oxopanasinsan-6-ol | 3-pinanone oxime |
| 4,4′-dimethoxydalbergione | 4-acetoxyphenol |
| 4-hydroxy-6-methylpyran-2-one | 4′-hydroxychalcone |
| 4′-methoxychalcone | 4-methoxydalbergione |
| 4′-methoxyflavone | 4-methylesculetin |
| 4-nonylphenol | 4-o-methylphloracetophenone |
| 5,2′-dimethoxyflavone | 5,7-dihydroxyflavone |
| 5,7-dihydroxyisoflavone | 5,7-dimethoxyisoflavone |
| 5alpha-androstan-3,17-dione | 5alpha-cholestan-3beta-ol-6-one |
| 5-hydroxy-2′,4′,7,8-tetramethoxyflavone | 5-hydroxyiminoisocaryophyllene |
| 6,3′-dimethoxyflavone | 6,4′-dihydroxyflavone |
| 6,4′-dimethoxyflavone | 6-acetoxyangolensic acid methyl ester |
| 6-hydroxyangolensic acid methyl ester | 7,2′-dihydroxyflavone |
| 7,2′-dimethoxyflavone | 7,4′-dihydroxyflavone |
| 7,8-dihydroxyflavone | 7-deacetoxy-7-oxokhivorin |
| 7-deacetylkhivorin | 7-desacetoxy-6,7-dehydrogedunin |
| 7-oxocallitrisic acid, methyl ester | 7-oxocholesterol |
| 7-oxocholesteryl acetate | 8,2′-dimethoxyflavone |
| 8beta-hydroxycarapin, 3,8-hemiacetal | 8-hydroxy-15,16-bisnor-11-labden-13-one |
| 8-hydroxycarapinic acid | 8-iodocatechin tetramethyl ether |
| abienol | abietic acid |
| abrine (l) | abscisic acid (cis,trans; +/−) |
| acacetin | acacetin diacetate |
| acetosyringone | acetyl isogambogic acid |
| aconitic acid | aconitine |
| adonitol | aesculin |
| agelasine (stereochemistry of diterpene unknown) | agmatine sulfate |
| ajmaline | ajmaline diacetate |
| aklavine hydrochloride | albizziine |
| alizarin | alpha-dihydrogedunol |
| alpha-hydroxydeoxycholic acid | alpha-mangostin |
| alpha-tochopherol | alpha-toxicarol |
| ambelline | amygdalin |
| anabasamine hydrochloride | anabasine hydrochloride |
| andirobin | andrographolide |
| androsta-1,4-dien-3,17-dione | anethole |
| angolensic acid, methyl ester | angolensin (r) |
| anhydrobrazilic acid | anisodamine |
| antheraxanthin | anthothecol |
| antiarol | aphyllic acid |
| apigenin | apiin |
| apiole | arabitol(d) |
| arbutin | arcaine sulfate |
| arecoline hydrobromide | artemisinin |
| artenimol | arthonioic acid |
| asarinin (−) | asarylaldehyde |
| asiatic acid | atranorin |
| auraptene | austricine |
| avocadane acetate | avocadanofuran |
| avocadene | avocadene acetate |
| avocadenofuran | avocadyne |
| avocadyne acetate | avocadynofuran |
| azadirachtin | azelaic acid |
| baccatin iii | baeomycesic acid |
| baicalein | batyl alcohol |
| benzyl isothiocyanate | berbamine hydrochloride |
| berberine chloride | bergapten |
| bergaptol | bergenin |
| beta-amyrin | beta-amyrin acetate |
| beta-caryophyllene alcohol | beta-dihydrogedunol |
| beta-dihydrorotenone | beta-escin |
| betaine hydrochloride | beta-mangostin |
| beta-peltatin | beta-sitosterol |
| beta-toxicarol | betulin |
| betulinic acid | bicuculline (+) |
| bilirubin | biochanin a |
| biochanin a diacetate | biochanin a, 7-methyl ether |
| biochanin a, dimethyl ether | bisabolol |
| bisabolol acetate | bixin |
| boldine | bovinocidin (3-nitropropionic acid) |
| brazilein | brazilin |
| brucine | bussein |
| byssochlamic acid | cadaverine tartrate |
| cadin-4-en-10-ol | cafestol acetate |
| caffeic acid | camphor (1r) |
| camptothecin | canavanine |
| cantharidin | caperatic acid |
| capsaicin | capsanthin |
| carapin | carapin-8(9)-ene |
| carminic acid | carnitine (dl) hydrochloride |
| carnosic acid | carnosine |
| carylophyllene oxide | caryophyllene [t(−)] |
| caryophyllenyl acetate | catechin pentaacetate |
| catechin tetramethylether | cearoin |
| cedrelone | cedrol |
| cedryl acetate | celastrol |
| cellobiose (d[+]) | centaurein |
| cephalosporin c sodium | cephalotaxine |
| cepharanthine | cevadine |
| chaulmoogric acid | chelidonine (+) |
| chlorogenic acid | cholecalciferol |
| cholest-5-en-3-one | cholestan-3beta,5alpha,6beta-triol |
| cholestan-3-one | cholestanone |
| cholesterol | cholesteryl acetate |
| cholic acid | cholic acid, methyl ester |
| chondrosine | chrysanthellin a |
| chrysanthemic acid | chrysanthemic acid, ethyl ester |
| chrysanthemyl alcohol | chrysarobin |
| chrysin | chrysophanol |
| chukrasin methyl ether | cianidanol |
| cinchonidine | cinchonine |
| cineole | citrinin |
| citropten | citrulline |
| clovanediol diacetate | colchiceine |
| colchicine | colforsin |
| conessine | coniferyl alcohol |
| coralyne chloride | cortisone |
| corynanthine | cosmosiin |
| cotarnine chloride | cotinine |
| coumarin | coumarinic acid methyl ether |
| crassin acetate | creatinine |
| crinamine | crustecdysone |
| cryptotanshinone | culmorin |
| curcumin | cytidine |
| cytisine | d,1-threo-3-hydroxyaspartic acid |
| daidzein | dalbergione |
| dalbergione, 4-methoxy-4′-hydroxy- | dantron |
| daunorubicin | deacetoxy-7-oxisogedunin |
| deacetoxy-7-oxogedunin | deacetylgedunin |
| decahydrogambogic acid | deguelin(−) |
| dehydro (11,12)ursolic acid lactone | dehydrorotenone |
| dehydrovariabilin | deltaline |
| demethylnobiletin | deoxyandirobin |
| deoxycholic acid | deoxygedunin |
| deoxygedunol acetate | deoxykhivorin |
| deoxysappanone b 7,3′-dimethyl ether acetate | deoxysappanone b 7,4′-dimethyl ether |
| deoxysappanone b trimethyl ether | derrubone |
| derrustone | desoxypeganine hydrochloride |
| diallyl sulfide | diallyl trisulfide |
| dictamnine | diffractaic acid |
| difucol hexamethyl ether | digitonin |
| digitoxin | digoxigenin |
| digoxin | dihydrocelastrol |
| dihydrocelastryl diacetate | dihydrodeoxygedunin |
| dihydrofissinolide | dihydrogambogic acid |
| dihydrogedunic acid, methyl ester | dihydrogedunin |
| dihydrojasmonic acid | dihydrojasmonic acid, methyl ester |
| dihydromunduletone | dihydromundulone |
| dihydromyristicin | dihydrorobinetin |
| dihydrorotenone | dihydrosamidin |
| dihydrotanshinone i | dimethyl caperatate |
| dimethyl gambogate | dimethylcaffeic acid |
| diosgenin | diosmetin |
| diphenylurea | diprotin a |
| djenkolic acid | duartin (−) |
| duartin, dimethyl ether | echinocystic acid |
| ellagic acid | embelin |
| emetine | emodic acid |
| emodin | enoxolone |
| entandrophragmin | epi(13)torulosol |
| epiafzelechin (2r,3r)(−) | epiafzelechin trimethyl ether |
| epiandrosterone | epicatechin |
| epicatechin pentaacetate | epicoprosterol |
| epigallocatechin | epoxy (1,11)humulene |
| epoxygedunin | ergosta-7,22-dien-3-one |
| ergosterol | ergosterol acetate |
| eriodyctol | erysolin |
| esculetin | esculin monohydrate |
| estragole | ethyl everninate |
| eudesmic acid | eugenitol |
| eugenol | eugenyl benzoate |
| euparin | eupatorin |
| eupatoriochromene | euphol |
| euphol acetate | euphorbiasteroid |
| evernic acid | everninic acid |
| evoxine | farnesol |
| felamidin | ferulic acid |
| fisetin | fissinolide |
| flavokawain b | folic acid |
| formononetin | fraxidin methyl ether |
| frequentin | friedelin |
| fucostanol | fumarprotocetraric acid |
| fusidic acid | galanthamine hydrobromide |
| gallic acid | gambogic acid |
| gambogic acid amide | gangaleoidin |
| gangleoidin acetate | garcinolic acid |
| gardenin b | garlicin |
| gedunin | gedunol |
| geneticin | genistein |
| genkwanin | gentisic acid |
| geraldol | geranylgeraniol |
| gibberellic acid | ginkgetin, k salt |
| ginkgolic acid | gitoxigenin |
| gitoxigenin diacetate | gitoxin |
| glucitol-4-gucopyanoside | glutathione |
| glycyrrhizic acid | gossypin |
| gossypol | grayanotoxin i |
| griseofulvic acid | griseofulvin |
| guaiazulene | guvacine hydrochloride |
| haematommic acid | haematommic acid, ethyl ester |
| haematoporphyrin dihydrochloride | haematoxylin |
| harmaline | harmalol hydrochloride |
| harmane | harmine |
| harmol hydrochloride | harpagoside |
| hecogenin | hecogenin acetate |
| hederacoside c | hederagenin |
| helenine | hematein |
| herniarin | hesperetin |
| hesperidin | heteropeucenin, methyl ether |
| hexamethylquercetagetin | hieracin |
| hinokitiol | homopterocarpin |
| humulene (alpha) | huperzine a |
| hydrocotarnine hydrobromide | hydroquinidine |
| hydroxyprogesterone | hymecromone methyl ether |
| hypoxanthine | icariin |
| ichthynone | imperatorin |
| indole-3-carbinol | inosine |
| inositol | iretol |
| iridin | irigenin trimethyl ether |
| irigenol | isobergaptene |
| isoeugenitol | isoferulic acid |
| isogedunin | isoginkgetin |
| isokobusone | isoliquiritigenin |
| isoosajin | isopeonol |
| isopimpinellin | isorotenone |
| isosafrole | isotectorigenin trimethyl ether |
| isotectorigenin, 7-methyl ether | juarezic acid |
| juglone | kainic acid |
| karanjin | khayanthone |
| khayasin | khayasin c |
| khellin | khivorin |
| kinetin | kobusone |
| kojic acid | koparin |
| koparin 2′-methyl ether | kuhlmannin |
| kynuramine | kynurenine |
| l(+/−)-alliin | lagochilin |
| lanosterol | lanosterol acetate |
| lapachol | lappaconitine |
| larixinic acid | larixol |
| larixol acetate | lathosterol |
| lawsone | lecanoric acid |
| leoidin | leoidin dimethyl ether |
| leucodin | leucopterin |
| ligustilide | limonin |
| linalool (+) | linamarin |
| liquiritigenin dimethyl ether | lithocholic acid |
| lobaric acid | lobeline hydrochloride |
| loganic acid | loganin |
| lomatin | lonchocarpic acid |
| lunarine | lupanine perchlorate |
| lupanyl acid hydrochloride | lupinine |
| lycopodine perchlorate | lycorine |
| maackiain | madecassic acid |
| mandelic acid, methyl ester | mangiferin |
| marmesin | marmesin acetate |
| medicarpin | melatonin |
| melezitose | menadione |
| menthol(−) | menthone |
| menthyl benzoate | merogedunin |
| metameconine | methyl coclaurine |
| methyl deoxycholate | methyl everninate |
| methyl orsellinate | methyl robustone |
| methylnorlichexanthone | methylorsellinic acid, ethyl ester |
| methylxanthoxylin | mexicanolide |
| mimosine | monocrotaline |
| morin | mucic acid |
| mundoserone | mundulone |
| mundulone acetate | muurolladie-3-one |
| naringenin | naringin |
| neotigogenin acetate | nerol |
| nerolidol | niloticin |
| n-methylanthranilic acid | n-methylisoleucine |
| nobiletin | nomilin |
| nonic acid | nopaline |
| nordihydroguaretic acid | noreleagnine |
| norharman | norstictic acid |
| obliquin | obtusaquinone |
| octopamine hydrochloride | odoratone |
| oleananoic acid acetate | oleanoic acid |
| oleanolic acid acetate | ononetin |
| orsellinic acid | orsellinic acid dimethyl ether |
| orsellinic acid, ethyl ester | osajin |
| osthol | ouabain |
| o-veratraldehyde | oxonitine |
| pachyrrhizin | pachyrrhizone |
| paclitaxel | paeonol |
| palmatine chloride | parthenolide |
| patulin | pectolinarin |
| pelletierine hydrochloride | penicillic acid |
| peoniflorin | perillic acid (−) |
| perillyl alcohol | perseitol |
| persitol heptaacetate | peucedanin |
| peucenin | phenacylamine hydrochloride |
| phloracetophenone | phloretin |
| phloridzin | p-hydroxycinnamaldehyde |
| physcion | phytol |
| picropodophyllotoxin | picropodophyllotoxin acetate |
| picrotin | picrotoxinin |
| pimpinellin | pinocembrin |
| pinosylvin methyl ether | piperine |
| piplartine | piscidic acid |
| plectocomine methyl ether | plumbagin |
| podofilox | podophyllotoxin acetate |
| podototarin | pomiferin |
| prenyletin | primuletin |
| pristimerin | pristimerol |
| protoporphyrin ix | pseudo-anisatin |
| ptaeroxylin | pterin-6-carboxylic acid |
| pteryxin | punctaporonin b |
| purpurin | purpurogallin |
| pyridoxine | pyrocatechuic acid |
| pyrromycin | quassin |
| quebrachitol | quercetin |
| quercetin pentamethyl ether | quercetin tetramethyl (5,7,3′,4′) ether |
| quercitrin | quinic acid |
| rauwolscine hydrochloride | reserpine |
| resveratrol | resveratrol 4′-methyl ether |
| retinol | retusin 7-methyl ether |
| retusoquinone | rhamnetin |
| rhapontin | rhetsinine |
| rhodinyl acetate | rhoifolin |
| robustic acid | robustone |
| roccellic acid | rockogenin |
| rosmarinic acid | rotenone |
| rotenonic acid | rubescensin a |
| rutilantinone | rutoside (rutin) |
| safrole | safrolglycol |
| salicin | salsolidine |
| salsoline | salvinorin a |
| salvinorin b | sanguinarine sulfate |
| santonin | sapindoside a |
| sappanone a dimethyl ether | sappanone a trimethyl ether |
| sarmentogenin | sarmentoside b |
| scandenin | scandenin diacetate |
| scopoletin | scopoline |
| securinine | selinidin |
| senecrassidiol 6-acetate | sennoside a |
| sericetin | shikimic acid |
| silibinin | sinapic acid methyl ether |
| sinensetin | s-isocorydine (+) |
| sitosteryl acetate | smilagenin |
| smilagenin acetate | solanesol |
| solanesyl acetate | solasodine |
| solidagenone | sparteine sulfate |
| sphondin | stictic acid |
| stigmasterol | strophanthidin |
| strychnine | sumaresinolic acid |
| tangeritin | tannic acid |
| tanshinone iia | tectorigenin |
| tetrahydrocortisone | tetrahydrogambogic acid |
| tetrahydropalmatine | tetrahydrosappanone a trimethyl ether |
| theaflavin | theanine |
| theobromine | thermopsine perchlorate |
| thiamine | thymoquinone |
| tigogenin | tomatidine hydrochloride |
| totaralolal | totarol |
| totarol acetate | totarol-19-carboxylic acid, methyl ester |
| triacetylresveratrol | tridesacetoxykhivorin |
| trigonelline | triptophenolide |
| tropine | tryptamine |
| tubaic acid | umbelliferone |
| ursinoic acid | ursocholanic acid |
| ursodiol | ursolic acid |
| usnic acid | utilin |
| uvaol | veratric acid |
| veratridine | vinblastine sulfate |
| vincamine | vincristine sulfate |
| vindoline | violastyrene |
| visnagin | vulpinic acid |
| xanthone | xanthopterin |
| xanthoxylin | xanthurenic acid |
| xanthyletin | xylocarpus a |
| yohimbine hydrochloride | zeorin |

### 6 REFERENCES CITED

All references cited herein are incorporated herein by reference in their entirety and for all purposes to the same extent as if each individual publication or patent or patent application was specifically and individually indicated to be incorporated by reference in its entirety herein for all purposes.

### 7 MODIFICATIONS

Many modifications and variations of the systems and methods disclosed herein can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed:

1. A method of searching for a combination of compounds of therapeutic interest, the method comprising:
(A) performing a first plurality of cell-based assays, each cell-based assay in the first plurality of cell-based assays comprising (i) exposing a different sample of cells to a different compound in a first plurality of compounds and (ii) measuring a phenotypic result in the different sample of cells upon exposure to the different compound thereby obtaining a first plurality of phenotypic results, each phenotypic result in the first plurality of phenotypic results corresponding to a compound in the first plurality of compounds;
(B) determining, from the first plurality of phenotypic results, a subset of compounds in the first plurality of compounds that implement a desired end-point phenotype;
(C) measuring, for each respective compound in the subset of compounds, a molecular abundance profile (MAP) using a different sample of cells that has been exposed to the respective compound thereby obtaining a first plurality of MAPs, each MAP in the first plurality of MAPs comprising cellular constituent abundance values for a plurality of cellular constituents in a sample of cells that has been exposed to a compound in the subset of compounds;
(D) determining a drug activity profile of each respective compound in the subset of compounds using (i) measured MAPs from the measuring (C) in which a sample of cells was exposed to the respective compound and (ii) an interaction network; and
(E) forming a filter set of compound combinations comprising a plurality compound combinations, each compound combination consisting of a combination of compounds in the subset of compounds, wherein a first compound and a second compound in a first compound combination in the plurality of compound combinations is selected from the subset of compounds based on a difference between a drug activity profile of the first compound and a drug activity profile of the second compound.

2. The method of claim 1, wherein the interaction network is determined using the MAPs from the measuring (C).

3. The method of claim 1, wherein a compound in the first plurality of compounds is used in single cell-based assay in the first plurality of cell-based assays at a single concentration.

4. The method of claim 1, wherein a compound in the first plurality of compounds is used in a first cell-based assay in the first plurality of cell-based assays at a first concentration and is used in a second cell-based assay in the first plurality of cell-based assay at a second concentration.

5. The method of claim 1, wherein a compound in the first plurality of compounds is used in a subset of cell-based assays in the first plurality of cell-based assays, wherein each cell-based assay in the subset of cell-based assays in which the compound is used is at a same or different concentration.

6. The method of claim 1, wherein each respective compound in the first plurality of compounds is used in a subset of cell-based assays in the first plurality of cell-based assays, wherein each cell-based assay in the subset of cell-based assays in which a respective compound is used is at a same or different concentration.

7. The method of claim 1, wherein a compound in the first plurality of compounds is assayed in a single cell-based assay in the first plurality of cell-based assays after exposure to a sample of cells for a period of time.

8. The method of claim 1, wherein a compound in the first plurality of compounds is assayed using a first aliquot of cells in a first cell-based assay in the first plurality of cell-based assays after exposure of the first aliquot of cells to the compound for a first duration t₁ and is assayed using a second aliquot of cells in a second cell-based assay in the first plurality of cell-based assays after exposure of the second aliquot of cells to the compound for a duration t₂, wherein the first aliquot of cells and the second aliquot of cells exhibit a phenotype of interest prior to exposure to the compound and duration t₁ is different then duration t₂.

9. The method of claim 1, wherein a compound in the first plurality of compounds is assayed in a plurality of cell-based assays in the first plurality of cell-based assays, wherein each cell-based assay in the plurality of cell-based assays in which the compound is used is assayed after a different aliquot of cells has been exposed to the compound for the same duration or for a different duration.

10. The method of claim 1, wherein each respective compound in the first plurality of compounds is assayed in a subset of cell-based assays in the first plurality of cell-based assays, wherein each cell-based assay in the subset of cell-based assays in which a respective compound is used is assayed after exposure to the compound for a same or different duration.

11. The method of claim 1, wherein the measuring (C) further comprises measuring, for each respective compound in a plurality of validated compounds, a MAP using a different sample of cells that has been exposed to the respective compound thereby obtaining a second plurality of MAPs, each MAP in the second plurality of MAPs comprising cellular constituent abundance values for a plurality of cellular constituents in a sample of cells that has been exposed to a compound in the plurality of validated compounds.

12. The method of claim 11, wherein the performing (A) further comprises performing a second plurality of cell-based assays, each cell-based assay in the second plurality of cell-based assays for a different compound in a plurality of validated compounds, each cell-based assay in the second plurality of cell-based assays comprising (i) exposing a different compound in the plurality of validated compounds to a different sample of cells, and (ii) measuring a phenotypic result of the different sample of cells upon exposure of the different compound, thereby obtaining a second plurality of phenotypic results, each phenotypic result in the second plurality of phenotypic results corresponding to a compound in the plurality of validated compounds.

13. The method of claim 12, wherein a compound in the plurality of validated compounds is used in single cell-based assay in the second plurality of cell-based assays at a single concentration.

14. The method of claim 12, wherein a compound in the plurality of validated compounds is used in a first cell-based assay in the second plurality of cell-based assays at a first concentration and is used in a second cell-based assay in the second plurality of cell-based assays at a second concentration.

15. The method of claim 12, wherein a compound in the plurality of validated compounds is used in a subject of cell-based assays in the second plurality of cell-based assays, wherein each cell-based assay in the subset of cell-based assays in which the compound is used is at a same or different concentration.

16. The method of claim 12, wherein each respective compound in the plurality of validated compounds is used in a subset of cell-based assays in the second plurality of cell-based assays, wherein each cell-based assay in the subset of cell-based assays in which a respective compound is used is at a same or different concentration.

17. The method of claim 1, wherein the interaction network comprises one or more transcriptional targets of each of one or more expressed transcription factors.

18. The method of claim 17, wherein the one or more transcriptional targets of each of the one or more expressed transcription factors are determined by identifying a gene-gene coregulation between a first cellular constituent in the plurality of cellular constituents that is a transcriptional target and a second cellular constituent in the plurality of cellular constituents that is a transcription factor from an information theoretic measure I(X; y) between a set of cellular constituent abundance values X for the first cellular constituent and a set of cellular constituent abundance values Y for the second cellular constituent, wherein
X={x₁, . . . , xₙ} and each Xᵢ in X is a cellular constituent abundance value for the first cellular constituent in a MAP i measured in the measuring (C),
Y={y₁, . . . , yₙ} and each Yᵢ in Y is a cellular constituent abundance value for the second cellular constituent in a MAP i measured in the measuring (C), and
n is an integer greater than one.

19. The method of claim 17, wherein the interaction network further comprises one or more transcription factor modulatory interactions caused by one or more post-translational modulators of transcription factor activity.

20. The method of claim 18, wherein the one or more post-translational modulators of transcription factor activity are caused by one or more cellular constituents in the plurality of cellular constituents that are post-translational modulators of transcription factor activity, the method further comprising identifying the one or more post-translational modulators from a plurality of MAPs measured in the measuring (C),
wherein, for a given post-translational modulator of transcription factor activity gₘ in the one or more post-translational modulators of transcription factor activity between a cellular constituent in the plurality of cellular constituents that is a transcription factor g_{TF} and a cellular constituent in the plurality of cellular constituents that is a target g_{T} of the transcription factor g_{TF}, the identifying comprises:
(i) partitioning a plurality of MAPs measured in the measuring (C) into a first microarray profile subset Lₘ⁺ and a second microarray profile subset Lₘ⁻ in which gₘ is respectively at its highest (gₘ⁺) and lowest (gₘ⁻) abundances in a plurality of MAPs measured in the measuring (C), wherein Lₘ⁻ and Lₘ⁺ are nonoverlapping and wherein Lm and Lₘ⁺ collectively encompass all or a portion of a plurality of MAPs measured in the measuring (C), and
(ii) identifying a conditional coregulation between g_{TF} and gₜ given gₘ by the conditional information difference ΔI(g_{TF},gₜ|gₘ) wherein
ΔI(g_{TF},gₜ|gₘ)=|I(g_{TF},gₜ|gₘ⁺)−I(g_{TF},gₜ|gₘ⁻)
and wherein
I(g_{TF},gₜ|gₘ⁻) is an information theoretic measure of an abundance of the transcription factor g_{TF} and an abundance of the target g_{T} across Lₘ⁺ given an abundance of the post-translational modulator of transcription factor activity gₘ across Lₘ⁺; and
I(G_{TF},gₜ|gₘ⁻) is an information theoretic measure of an abundance of the transcription factor g_{TF} and an abundance of the target g_{T} across Lₘ⁻ given an abundance of the post-translational modulator of transcription factor activity gₘ across Lₘ⁻.

21. The method of claim 1, the method further comprising:
(F) screening a subset of compound combinations in the filter set of compound combinations for the ability to cause the desired end-point phenotype.

22. The method of claim 1, the method further comprising:
(F) outputting the filter set of compound combinations in a format accessible to a user, to a computer readable memory, to a tangible computer readable media, to a local or remote computer system, or to a display.

23. The method of claim 1, wherein the first plurality of compounds comprises one thousand compounds or more.

24. The method of claim 1, wherein the first plurality of compounds comprises ten thousand compounds or more.

25. The method of claim 1, wherein the first plurality of compounds comprises one hundred thousand compounds or more.

26. The method of claim 1, wherein
the exposing (i) of (A) comprises exposing the different compound to a sample of cells that is malignant and exposing the different compound to a sample of cells that is not malignant; and
the phenotypic result is a relative end-point effect of (a) the sample of cells that is malignant upon exposure to the different compound and (b) the sample of cells that is not malignant upon exposure to the different compound in the plurality compounds.

27. The method of claim 1, wherein
the exposing (i) of (A) comprises exposing the different compound to a sample of cells that exhibits a phenotype of interest and exposing the different compound to a sample of cells that does not exhibit the phenotype of interest; and
the phenotypic result is a relative end-point effect of (a) the sample of cells that is malignant upon exposure to the different compound and (b) the sample of cells that is not malignant upon exposure to the different compound.

28. The method of claim 1, wherein the exposing (i) of (A) comprises exposing the different compound to a plurality of different cells lines, wherein at least one cell line in the plurality of different cell lines exhibits a phenotype of interest and at least one cell line in the plurality of different cell lines does not exhibit the phenotype of interest.

29. The method of claim 1, wherein a different sample of cells used in the performing (A) exhibits a cancerous.

30. The method of claim 1, wherein a different sample of cells used in the performing (A) is derived from a bladder cancer sample, a breast cancer sample, a colorectal cancer sample, a gastric cancer sample, a germ cell cancer sample, a kidney cancer sample, a hepatocellular cancer sample, a non-small cell lung cancer sample, a non-Hodgkin's lymphoma sample, a melanoma sample, an ovarian cancer sample, a pancreatic cancer sample, a prostate cancer sample, a soft tissue sarcoma sample, or a thyroid cancer sample.

31. The method of claim 1, wherein the plurality of cellular constituents is between 5 mRNAs and 50,000 mRNAs and the cellular constituent abundance values are amounts of each mRNA.

32. The method of claim 1, wherein the plurality of cellular constituents is between 50 proteins and 200,000 proteins and the cellular constituent abundance values are amounts of each protein.

33. The method of claim 1, wherein the interaction network comprises an identity of the cellular constituents in the plurality of cellular constituents and a plurality of edges wherein each edge connects two cellular constituents in the plurality of cellular constituents in a directed or undirected manner, wherein each edge represents a protein-protein interaction, a protein-DNA interaction or a transcription factor modulatory interaction.

34. The method of claim 1, wherein
the exposing (i) of the performing (A) comprises exposing the different compound to a different sample of cells that exhibits a phenotype of interest and exposing the different compound to a different sample of cells that does not exhibit the phenotype of interest;
the measuring (C) comprises (i) measuring a MAP of the different sample of cells that exhibits the phenotype of interest after exposure to the different compound and (ii) measuring a MAP of the different sample of cells that does not exhibit the phenotype of interest after exposure to the different compound; and
the determining (D) for a compound in the subset of compounds comprises identifying each respective edge between a cellular constituent that is a transcription factor a and a cellular constituent that is a transcription factor target b that exhibits loss of correlation (LoC) or gain of correlation (GoC) based on an estimate of the information difference ΔI, wherein
ΔI=I_{AH}[A;B]−I_{AH-P}[A;B]
wherein,
I_{AH}[A;B] is an information theoretic measure between cellular constituent abundance values A for the transcription factor a, wherein each Ai in the set A={a₁, . . . , aₙ} is a value for the transcription factor a in a microarray sample measured in the measuring (C) and each Bᵢ in the set B={b₁, . . . , bₙ} is a cellular constituent abundance value for the transcription factor target b in a microarray sample measured in the measuring (C), and
I_{AH-P}[A;B] is an information theoretic measure between cellular constituent abundance values A for the transcription factor a in each of a plurality of microarray samples measured in the measuring (C) not taken from samples of cells exhibiting the phenotype of interest and cellular constituent abundance values B for the transcription factor target b in a plurality of microarray samples measured in the measuring (C) not taken from samples of cells exhibiting the phenotype of interest.

35. The method of claim 34, wherein the determining (D) further comprises identifying a drug activity profile of a compound in the subset of compounds as those cellular constituents in the interaction network that are statistically enriched for LoC and/or GoC interactions.

36. The method of claim 34, wherein the information theoretic measure is mutual information or a correlation.

37. The method of claim 1, wherein the forming (E) comprises selecting a first compound from the subset of compounds for inclusion in a compound combination in the filter set of compound combinations when
(i) exposure of the first compound to the different sample of cells in the performing (A) achieves the desired end-point phenotype in the different sample of cells;
(ii) the first compound has a drug activity profile that comprises one or more cellular constituents that are not in a drug activity profile of a second compound that achieves the desired end-point phenotype in a cell line upon exposure of the cell line to the second compound; or
(iii) the first compound is designed to specifically inhibit a cellular constituent that is not in the drug activity profile of the second compound.

38. The method of claim 1, wherein each compound combination in the filter set of compound combinations consists of two different compounds in the subset of compounds.

39. The method of claim 1, wherein each compound combination in the filter set of compound combinations consists of three different compounds in the subset of compounds.

40. The method of claim 1, wherein the filter set of compound combinations comprises 10,000 or more compound combinations.

41. The method of claim 1, wherein the filter set of compound combinations comprises 50,000 or more compound combinations.

42. The method of claim 21, wherein the screening (F) comprises performing a plurality of cell-based confirmation assays, each cell-based confirmation assay in the plurality of cell-based confirmation assays comprising:
(i) exposing a different compound combination in the filter set of compound combinations to a different sample of cells, and
(ii) measuring a phenotypic result of the different sample of cells upon exposure of the different compound combination.

43. The method of claim 42, wherein the phenotypic result is cell death as a function of an amount of a compound in the different compound composition.

44. The method of claim 1, wherein the performing (A) comprises assessing the phenotypic result using an automated fluorescent or luminescent readout with a robotically integrated plate-reader.

45. The method of claim 44, wherein the phenotypic result is measured using an automated fluorescent or luminescent readout with a robotically integrated plate-reader.

46. The method of claim 18, wherein the information theoretic measure I(X;Y) is the mutual information of X and Y.

47. The method of claim 20, wherein the interaction network is formed using a Bayesian analysis of the one or more transcriptional targets of each of one or more expressed transcription factors and one or more transcription modulator interactions caused by one or more post-translational modulators of transcription factor activity.

48. The method of claim 1, wherein the different sample of cells tested in the performing (A) is from a predetermined human tissue type.

49. The method of claim 48, wherein the predetermined human tissue type is heart, lung, brain, pancreas, liver, or breast.

50. The method of claim 1, the method further comprising:
(i) computing a cellular constituent signature of the desired end-point phenotype, wherein the cellular constituent signature of the desired end-point phenotype comprises differences in cellular constituent abundance values of each cellular constituent in a plurality of cellular constituents between (a) a cell sample exhibiting a phenotype of interest and (b) a cell sample that exhibits the phenotype of interest and that also exhibits the desired end-point phenotype;
(ii) determining, using the cellular constituent signature of the desired end-point phenotype as well as the interaction network, a plurality of transcription factors that can cause the desired end-point phenotype; and wherein
the drug activity profile, for each respective compound in the subset of compounds, indicates whether the respective compound affects an abundance of one or more transcription factors in the plurality of transcription factors as determined by the interaction network and a differential profile of the respective compound, wherein the differential profile of the respective compound comprises differences in cellular constituent abundance values of each cellular constituent in a plurality of cellular constituents between (i) cells that have not been exposed to the respective compound and (ii) cells that have been exposed to the respective compound; and
the forming (E) comprises selecting a compound combination for the filter set of compound combinations based on a combination of (i) a drug activity profile of each compound in the compound combination as determined in the determining (D), and (ii) a difference in the differential profile of each compound in the compound combination.

51. The method of claim 1, the method further comprising:
(i) computing a cellular constituent signature of the desired end-point phenotype, wherein the cellular constituent signature of the desired end-point phenotype comprises differences in cellular constituent abundance values of each cellular constituent in a plurality of cellular constituents between (a) a cell sample exhibiting a phenotype of interest and (b) a cell sample exhibiting that phenotype of interest that also exhibits the desired end-point phenotype;
(ii) determining, using the cellular constituent signature of the desired end-point phenotype as well as the interaction network, a plurality of post-translational modulators of transcription factor activity that can implement the desired end-point phenotype; and wherein
the drug activity profile, for each respective compound in the subset of compounds, indicates whether the respective compound affects an abundance of one or more post-translational modulators of transcription factor activity in the plurality of post-translational modulators of transcription factor activity as determined by the interaction network and a differential profile of the respective compound, wherein the differential profile of the respective compound comprises differences in cellular constituent abundance values of each cellular constituent in a plurality of cellular constituents between (i) cells that have not been exposed to the respective compound and (ii) cells that have been exposed to the respective compound; and
the forming (E) comprises selecting a compound combination for the filter set of compound combinations based on a combination of (i) a drug activity profile of each compound in the compound combination as determined in the determining (D), and (ii) a difference in the differential profile of each compound in the compound combination.

52. A method of searching for a combination of compounds of therapeutic interest, the method comprising:
(A) performing a first plurality of cell-based assays, each cell-based assay in the first plurality of cell-based assays comprising (i) exposing a different compound in a first plurality of compounds to a different sample of cells and (ii) measuring a phenotypic result of the different sample of cells upon exposure of the different compound thereby obtaining a first plurality of phenotypic results, each phenotypic result in the first plurality of phenotypic results corresponding to a compound in the first plurality of compounds;
(B) determining, from the first plurality of phenotypic results, a subset of compounds in the first plurality of compounds that can causes a desired end-point phenotype;
(C) measuring, for each respective compound in the subset of compounds, a molecular abundance profile (MAP) using a different sample of cells that has been exposed to the respective compound thereby obtaining a first plurality of MAPs, each MAP in the first plurality of MAPs comprising cellular constituent abundance values for a plurality of cellular constituents in a sample of cells that has been exposed to a compound in the subset of compounds;
(D) computing, for each respective compound in the subset of compounds, a compound similarity score between (i) a differential profile of the respective compound and (ii) a cellular constituent signature of the desired end-point phenotype, thereby calculating a plurality of compound similarity scores; wherein
the differential profile of the respective compound comprises differences in cellular constituent abundance values of each cellular constituent in a plurality of cellular constituents between (i) cells that have not been exposed to the respective compound and (ii) cells that have been exposed to the respective compound; and
the cellular constituent signature of the desired end-point phenotype comprises differences in cellular constituent abundance values of each cellular constituent in a plurality of cellular constituents between (i) a cell sample representative of a phenotype of interest and (ii) a cell sample that is representative of a phenotype of interest and that is also exhibiting the desired end-point phenotype; and
(E) forming a filter set of compound combinations comprising a plurality compound combinations, each compound combination consisting of a combination of compounds in the subset of compounds, wherein a compound combination in the plurality of compound combinations is selected based on a combination of (i) a compound similarity score of each compound in the compound combination as determined in the computing (D), and (ii) a difference in the differential profile of each compound, determined in the computing (D), in the compound combination.

53. The method of claim 52, wherein a compound in the first plurality of compounds is used in single cell-based assay in the first plurality of cell-based assays at a single concentration.

54. The method of claim 52, wherein a compound in the first plurality of compounds is used in a first cell-based assay in the first plurality of cell-based assays at a first concentration and is used in a second cell-based assay in the first plurality of cell-based assay at a second concentration.

55. The method of claim 52, wherein a compound in the first plurality of compounds is used in a subset of cell-based assays in the first plurality of cell-based assays, wherein each cell-based assay in the subset of cell-based assays in which the compound is used is at a same or different concentration.

56. The method of claim 52, wherein each respective compound in the first plurality of compounds is used in a subset of cell-based assays in the first plurality of cell-based assays, wherein each cell-based assay in the subset of cell-based assays in which a respective compound is used is at a same or different concentration.

57. The method of claim 52, wherein a compound in the first plurality of compounds is assayed in single cell-based assay in the first plurality of cell-based assays upon exposure of an aliquot of cells to the compound for a single time duration.

58. The method of claim 52, wherein a compound in the first plurality of compounds is assayed in a first cell-based assay in the first plurality of cell-based assays upon exposure of a first aliquot of cells to the compound for a first duration of time and is assayed in a second cell-based assay in the first plurality of cell-based assay f upon exposure of a second aliquot of cells to the compound for a second duration of time, wherein the first duration of time is different then the second duration of time.

59. The method of claim 52, wherein a compound in the first plurality of compounds is assayed in a subset of cell-based assays in the first plurality of cell-based assays, wherein each cell-based assay in the plurality of cell-based assays in which the compound is used is assayed after exposure of a different aliquot of cells to the compound for a different duration of time.

60. The method of claim 52, wherein each respective compound in the first plurality of compounds is assayed in a subset of cell-based assays in the first plurality of cell-based assays, wherein each cell-based assay in the plurality of cell-based assays in which a respective compound is used is assayed after exposure of a different aliquot of cells to the compound for a same or different duration of time.

61. The method of claim 52, wherein the measuring (C) further comprises measuring, for each respective compound in a plurality of validated compounds, a MAP using a different sample of cells that has been exposed to the respective compound thereby obtaining a second plurality of MAPs, each MAP in the second plurality of MAPs comprising cellular constituent abundance values for a plurality of cellular constituents in a sample of cells that has been exposed to a compound in the plurality of validated compounds.

62. The method of claim 61, wherein the performing (A) further comprises performing a second plurality of cell-based assays, each cell-based assay in the second plurality of cell-based assays for a different compound in a plurality of validated compounds, each cell-based assay in the second plurality of cell-based assays comprising (i) exposing a different compound in the plurality of validated compounds to a different sample of cells, and (ii) measuring a phenotypic result of the different sample of cells upon exposure of the different compound, thereby obtaining a second plurality of phenotypic results, each phenotypic result in the second plurality of phenotypic results corresponding to a compound in the plurality of validated compounds.

63. The method of claim 62, wherein a compound in the plurality of validated compounds is used in single cell-based assay in the second plurality of cell-based assays at a single concentration.

64. The method of claim 62, wherein a compound in the plurality of validated compounds is used in a first cell-based assay in the second plurality of cell-based assays at a first concentration and is used in a second cell-based assay in the second plurality of cell-based assays at a second concentration.

65. The method of claim 62, wherein a compound in the plurality of validated compounds is used in a plurality of cell-based assays in the second plurality of cell-based assays, wherein each cell-based assay in the plurality of cell-based assays in which the compound is used is at a same or different concentration.

66. The method of claim 62, wherein each respective compound in the plurality of validated compounds is used in a plurality of cell-based assays in the second plurality of cell-based assays, wherein each cell-based assay in the plurality of cell-based assays in which a respective compound is used is at a same or different concentration.

67. The method of claim 52, the method further comprising:
(F) screening a subset of compound combinations in the filter set of compound combinations for the ability to cause the desired end-point phenotype in a cell based assay.

68. The method of claim 52, the method further comprising:
(F) outputting the filter set of compound combinations in a format accessible to a user, to a computer readable memory, to a tangible computer readable media, to a local or remote computer system, or to a display.

69. The method of claim 52, wherein the first plurality of compounds comprises one thousand compounds or more.

70. The method of claim 52, wherein the first plurality of compounds comprises ten thousand compounds or more.

71. The method of claim 52, wherein the first plurality of compounds comprises one hundred thousand compounds or more.

72. The method of claim 52, wherein
the exposing (i) of the performing (A) comprises exposing the different compound to a sample of cells that is malignant and exposing the different compound to a sample of cells that is not malignant; and
the phenotypic result is a relative end-point effect of (a) the sample of cells that is malignant upon exposure to the different compound and (b) the sample of cells that is not malignant upon exposure to the different compound in the plurality compounds.

73. The method of claim 52, wherein
the exposing (i) of the performing (A) comprises exposing the different compound to a sample of cells that exhibits the phenotype of interest and exposing the different compound to a sample of cells that does not exhibit the phenotype of interest; and
the phenotypic result is a relative end-point effect of (a) the sample of cells that is malignant upon exposure to the different compound and (b) the sample of cells that is not malignant upon exposure to the different compound.

74. The method of claim 52, wherein the exposing (i) of the performing (A) comprises exposing the different compound to a plurality of different cells lines, wherein at least one cell line in the plurality of different cell lines exhibits the phenotype of interest and at least one cell line in the plurality of different cell lines does not exhibit the phenotype of interest.

75. The method of claim 52, wherein the phenotype of interest is a disease.

76. The method of claim 52, wherein the phenotype of interest is a cancer.

77. The method of claim 52, wherein the phenotype of interest is bladder cancer, breast cancer, colorectal cancer, gastric cancer, germ cell cancer, kidney cancer, hepatocellular cancer, non-small cell lung cancer, non-Hodgkin's lymphoma, melanoma, ovarian cancer, pancreatic cancer, prostate cancer, soft tissue sarcoma, or thyroid cancer.

78. The method of claim 52, wherein the plurality of cellular constituents is between 5 mRNAs and 50,000 mRNAs and the cellular constituent abundance values are amounts of each mRNA.

79. The method of claim 52, wherein the plurality of cellular constituents is between 50 proteins and 200,000 proteins and the cellular constituent abundance values are amounts of each protein.

80. The method of claim 52, wherein each compound combination in the filter set of compound combinations consists of two different compounds in the subset of compounds.

81. The method of claim 52, wherein each compound combination in the filter set of compound combinations consists of three different compounds in the subset of compounds.

82. The method of claim 52, wherein the filter set of compound combinations comprises 10,000 or more compound combinations.

83. The method of claim 52, wherein the filter set of compound combinations comprises 50,000 or more compound combinations.

84. The method of claim 67, wherein the screening (F) comprises performing a plurality of cell-based confirmation assays, each cell-based confirmation assay in the plurality of cell-based confirmation assays comprising:
(i) exposing a different compound combination in the filter set of compound combinations to a different sample of cells, and
(ii) measuring a phenotypic result of the different sample of cells upon exposure of the different compound combination.

85. The method of claim 84, wherein the phenotypic result is cell death as a function of an amount of a compound in the different compound composition.

86. The method of claim 52, wherein the performing (A) comprises assessing the phenotypic result using an automated fluorescent or luminescent readout with a robotically integrated plate-reader.

87. The method of claim 86, wherein the phenotypic result is measured using an automated fluorescent or luminescent readout with a robotically integrated plate-reader.

88. The method of claim 52, wherein the different sample of cells tested in the performing (A) is representative of a predetermined human tissue type.

89. The method of claim 88, wherein the predetermined human tissue type is heart, lung, brain, pancreas, liver, or breast.

90. The method of claim 52, the method further comprising outputting the filter set of compounds to a user, a computer readable memory, a computer readable media, or a display.

91. An apparatus for searching for a combination of compounds of therapeutic interest, the apparatus comprising:
a processor; and
a memory, coupled to the processor, the memory storing one or more modules that individually or collectively comprise instructions, executable by the processor, for:
(A) receiving a first plurality of phenotypic results, wherein each phenotypic result in the first plurality of phenotypic results from (i) exposing a different sample of cells to a different compound in a first plurality of compounds and (ii) measuring a phenotypic result in the different sample of cells upon exposure of the different compound, each phenotypic result in the first plurality of phenotypic results corresponding to a compound in the first plurality of compounds;
(B) determining, from the first plurality of phenotypic results, a subset of compounds in the first plurality of compounds that implement a desired end-point phenotype;
(C) receiving, for each respective compound in the subset of compounds, a molecular abundance profile (MAP) that is measured using a different sample of cells that has been exposed to the respective compound, thereby receiving a first plurality of MAPs, each MAP in the first plurality of MAPs comprising cellular constituent abundance values for a plurality of cellular constituents in a sample of cells that has been exposed to a compound in the subset of compounds;
(D) determining a drug activity profile of each respective compound in the subset of compounds using (i) measured MAPs from the instructions for receiving (C) in which the respective compound was exposed to a sample of cells and (ii) an interaction network; and
(E) forming a filter set of compound combinations comprising a plurality compound combinations, each compound combination consisting of a combination of compounds in the subset of compounds, wherein a first compound and a second compound in a first compound combination in the plurality of compound combinations is selected from the subset of compounds based on a difference between a drug activity profile of the first compound and a drug activity profile of the second compound.

92. The apparatus of claim 91, wherein the one or more modules further individually or collectively comprise instructions, executable by the processor, for outputting the filter set of compound combinations to a user, a computer readable memory, a computer readable media, a local or remote computer system, or a display.

93. A computer-readable medium storing one or more computer programs executable by a computer for searching a combination of compounds of therapeutic interest, the one or more computer programs individually or collectively comprising computer executable instructions for:
(A) receiving a first plurality of phenotypic results, wherein each phenotypic result in the first plurality of phenotypic results from (i) exposing a different sample of cells to a different compound in a first plurality of compounds and (ii) measuring a phenotypic result of the different sample of cells upon exposure to the different compound, each phenotypic result in the first plurality of phenotypic results corresponding to a compound in the first plurality of compounds;
(B) determining, from the first plurality of phenotypic results, a subset of compounds in the first plurality of compounds that implements a desired end-point phenotype;
(C) receiving, for each respective compound in the subset of compounds, a molecular abundance profile (MAP) that is measured using a different sample of cells that has been exposed to the respective compound, thereby receiving a first plurality of MAPs, each MAP in the first plurality of MAPs comprising cellular constituent abundance values for a plurality of cellular constituents in a sample of cells that has been exposed to a compound in the subset of compounds;
(D) determining a drug activity profile of each respective compound in the subset of compounds using (i) measured MAPs from the instructions for receiving (C) in which a sample of cells was exposed to the respective compound and (ii) an interaction network; and
(E) forming a filter set of compound combinations comprising a plurality compound combinations, each compound combination consisting of a combination of compounds in the subset of compounds, wherein a first compound and a second compound in a first compound combination in the plurality of compound combinations is selected from the subset of compounds based on a difference between a drug activity profile of the first compound and a drug activity profile of the second compound.

94. The computer-readable medium of claim 93, wherein the one or more computer programs individually or collectively further comprise computer executable instructions for outputting the filter set of compound combinations to a user, a computer readable memory, a computer readable media, a local or remote computer system, or to a display.

95. An apparatus for searching for a combination of compounds of therapeutic interest, the apparatus comprising:
a processor; and
a memory, coupled to the processor, the memory storing one or more modules that individually or collectively comprise instructions, executable by the processor, for:
(A) receiving a first plurality of phenotypic results, each phenotypic result in the first plurality of phenotypic results from (i) exposing a different sample of cells to a different compound in a first plurality of compounds and (ii) measuring the phenotypic result in the different sample of cells upon exposure of the different compound, each phenotypic result in the first plurality of phenotypic results corresponding to a compound in the first plurality of compounds;
(B) determining, from the first plurality of phenotypic results, a subset of compounds in the first plurality of compounds that implement a desired end-point phenotype;
(C) receiving a molecular abundance profile (MAP), for each respective compound in the subset of compounds, wherein the MAP is measured using a different sample of cells that has been exposed to the respective compound, thereby obtaining a first plurality of MAPs, each MAP in the first plurality of MAPs comprising cellular constituent abundance values for a plurality of cellular constituents in a sample of cells that has been exposed to a compound in the subset of compounds;
(D) computing, for each respective compound in the subset of compounds, a compound similarity score between (i) a differential profile of the respective compound and (ii) a cellular constituent signature of a desired end-point phenotype, thereby calculating a plurality of compound similarity scores; wherein
the differential profile of the respective compound comprises differences in cellular constituent abundance values of each cellular constituent in a plurality of cellular constituents between (i) cells that have not been exposed to the respective compound and (ii) cells that have been exposed to the respective compound; and
the cellular constituent signature of the desired end-point phenotype comprises differences in cellular constituent abundance values of each cellular constituent in a plurality of cellular constituents between (i) a cell sample representative of a phenotype of interest and (ii) a cell sample representative of the desired end-point phenotype; and
(E) forming a filter set of compound combinations comprising a plurality compound combinations, each compound combination consisting of a combination of compounds in the subset of compounds, wherein a compound combination in the plurality of compound combinations is selected based on a combination of (i) a compound similarity score of each compound in the compound combination as determined in the computing (D), and a difference in the differential profile of each compound, determined in the computing (D), in the compound combination.

96. The apparatus of claim 95, wherein the one or more modules that individually or collectively comprise instructions, executable by the processor, further comprise instructions for outputting the filter set of compound combinations to a user, a computer readable memory, a computer readable media, a local or remote computer system, or a display.

97. A computer-readable medium storing one or more computer programs executable by a computer for searching a combination of compounds of therapeutic interest, the one or more computer programs individually or collectively comprising computer executable instructions for:
(A) receiving a first plurality of phenotypic results, each phenotypic result in the first plurality of phenotypic results from (i) exposing a different sample of cells to a different compound in a first plurality of compounds and (ii) measuring a phenotypic result of the different sample of cells upon exposure of the different compound, each phenotypic result in the first plurality of phenotypic results corresponding to a compound in the first plurality of compounds;
(B) determining, from the first plurality of phenotypic results, a subset of compounds in the first plurality of compounds that implement a desired end-point phenotype;
(C) receiving a molecular abundance profile (MAP), for each respective compound in the subset of compounds, wherein the MAP is measured using a different sample of cells that has been exposed to the respective compound, thereby obtaining a first plurality of MAPs, each MAP in the first plurality of MAPs comprising cellular constituent abundance values for a plurality of cellular constituents in a sample of cells that has been exposed to a compound in the subset of compounds;
(D) computing, for each respective compound in the subset of compounds, a compound similarity score between (i) a differential profile of the respective compound and (ii) a cellular constituent signature of the desired end-point phenotype, thereby calculating a plurality of compound similarity scores; wherein
the differential profile of the respective compound comprises differences in cellular constituent abundance values of each cellular constituent in a plurality of cellular constituents between (i) cells that have not been exposed to the respective compound and (ii) cells that have been exposed to the respective compound; and
the cellular constituent signature of the desired end-point phenotype comprises differences in cellular constituent abundance values of each cellular constituent in a plurality of cellular constituents between (i) a cell sample representative of a phenotype of interest and (ii) a cell sample representative of the desired end-point phenotype; and
(E) forming a filter set of compound combinations comprising a plurality compound combinations, each compound combination consisting of a combination of compounds in the subset of compounds, wherein a compound combination in the plurality of compound combinations is selected based on a combination of (i) a compound similarity score of each compound in the compound combination as determined in the computing (D), and a difference in the differential profile of each compound, determined in the computing (D), in the compound combination.

98. The computer-readable medium of claim 97, where the one or more computer programs individually or collectively further comprise computer executable instructions for outputting the filter set of compound combinations to a user, a computer readable memory, a computer readable media, a local or remote computer system, or a display.

99. The method of claim 1, wherein the phenotypic result that is measured is a determination as to whether or not the different sample of cells is undergoing apotosis and the desired end-point phenotype is cell apotosis.

100. The method of claim 1, wherein the phenotypic result that is measured is a determination as to whether or not the different sample of cells is undergoing cell proliferation and the desired end-point phenotype is cell proliferation.

101. The method of claim 1, wherein the phenotypic result that is measured is a determination as to whether or not a predetermined molecular event is occurring in the different sample of cells and the desired end-point phenotype is the occurrence of the predetermined molecular event.

102. The method of claim 101 wherein the predetermined molecular event is a predetermined conformational change of a protein of interest in the different sample of cells.

103. The method of claim 101 wherein the predetermined molecular event is a cellular localization of a protein of interest in the different sample of cells.

104. The method of claim 101 wherein
the phenotypic result that is measured by a FRET signal, a luciferase signal, or a reporter signal; and
the predetermined molecular event is deemed to have occurred upon an appearance of a FRET signal, a luciferase signal, or a reporter signal.

105. The method of claim 101 wherein
the phenotypic result that is measured by a FRET signal, a luciferase signal, or a reporter signal; and
the predetermined molecular event is deemed to have occurred upon a disappearance of a FRET signal, a luciferase signal, or a reporter signal.

106. The method of claim 101 wherein
the phenotypic result that is measured by a FRET signal, a luciferase signal, or a reporter signal; and
the predetermined molecular event is deemed to have occurred upon an attenuation a FRET signal, a luciferase signal, or a reporter signal.

107. The method of claim 101 wherein
the phenotypic result that is measured by a FRET signal, a luciferase signal, or a reporter signal; and
the predetermined molecular event is deemed to have occurred upon a deattenuation a FRET signal, a luciferase signal, or a reporter signal.

108. The method of claim 101 wherein
the phenotypic result that is measured by a FRET signal, a luciferase signal, or a reporter signal; and
the predetermined molecular event is deemed to have occurred upon a measurement of a FRET signal, a luciferase signal, or a reporter signal above a threshold value.

109. The method of claim 52, wherein
the phenotypic result that is measured by a FRET signal, a luciferase signal, or a reporter signal; and
the predetermined molecular event is deemed to have occurred upon a measurement of a FRET signal, a luciferase signal, or a reporter signal below a threshold value.

110. The method of claim 52, wherein the phenotypic result that is measured is a determination as to whether or not the different sample of cells is undergoing apotosis and the desired end-point phenotype is cell apotosis.

111. The method of claim 52, wherein the phenotypic result that is measured is a determination as to whether or not the different sample of cells is undergoing cell proliferation and the desired end-point phenotype is cell proliferation.

112. The method of claim 52, wherein the phenotypic result that is measured is a determination as to whether or not a predetermined molecular event is occurring in the different sample of cells and the desired end-point phenotype is the occurrence of the predetermined molecular event.

113. The method of claim 112, wherein the predetermined molecular event is a predetermined conformational change of a protein of interest in the different sample of cells.

114. The method of claim 112, wherein the predetermined molecular event is a cellular localization of a protein of interest in the different sample of cells.

115. The method of claim 112, wherein
the phenotypic result that is measured by a FRET signal, a luciferase signal, or a reporter signal; and
the predetermined molecular event is deemed to have occurred upon an appearance of a FRET signal, a luciferase signal, or a reporter signal.

116. The method of claim 112, wherein
the phenotypic result that is measured by a FRET signal, a luciferase signal, or a reporter signal; and
the predetermined molecular event is deemed to have occurred upon a disappearance of a FRET signal, a luciferase signal, or a reporter signal.

117. The method of claim 112, wherein
the phenotypic result that is measured by a FRET signal, a luciferase signal, or a reporter signal; and
the predetermined molecular event is deemed to have occurred upon an attenuation a FRET signal, a luciferase signal, or a reporter signal.

118. The method of claim 112, wherein
the phenotypic result that is measured by a FRET signal, a luciferase signal, or a reporter signal; and
the predetermined molecular event is deemed to have occurred upon a deattenuation a FRET signal, a luciferase signal, or a reporter signal.

119. The method of claim 112, wherein
the phenotypic result that is measured by a FRET signal, a luciferase signal, or a reporter signal; and
the predetermined molecular event is deemed to have occurred upon a measurement of a FRET signal, a luciferase signal, or a reporter signal above a threshold value.

120. The method of claim 112, wherein
the phenotypic result that is measured by a FRET signal, a luciferase signal, or a reporter signal; and
the predetermined molecular event is deemed to have occurred upon a measurement of a FRET signal, a luciferase signal, or a reporter signal below a threshold value.
